SEQUENCE LISTING (33 sequences)

SEQ ID NO: 1 - protein, 5 aa, synthetic construct
SYAMN

SEQ ID NO: 2 - protein, 17 aa, synthetic construct
GISSSGRYTGYADSVKG

SEQ ID NO: 3 - protein, 18 aa, synthetic construct
SVGSGVSWSGYVATSLDA

SEQ ID NO: 4 - protein, 8 aa, synthetic construct
SAGSGLYG

SEQ ID NO: 5 - protein, 7 aa, synthetic construct
GTNKRPS

SEQ ID NO: 6 - protein, 11 aa, synthetic construct
GSADSSTNAGI

SEQ ID NO: 7 - protein, 127 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSS

SEQ ID NO: 8 - protein, 105 aa, synthetic construct
SYELTQPPSVSVSPGQTARITCSAGSGLYGWYQQKPGQAPVLVIYGTNKRPSGIPERFSGSSSGTTVTLTISGVQAEDEADYYCGSADSSTNAGIFGGGTKLTVL

SEQ ID NO: 9 - protein, 5 aa, synthetic construct
TYAMN

SEQ ID NO: 10 - protein, 19 aa, synthetic construct
RIRSKYNNYATYYADSVKG

SEQ ID NO: 11 - protein, 14 aa, synthetic construct
HGNFGDSYVSWFAY

SEQ ID NO: 12 - protein, 14 aa, synthetic construct
GSSTGAVTTSNYAN

SEQ ID NO: 13 - protein, 7 aa, synthetic construct
GTNKRAP

SEQ ID NO: 14 - protein, 9 aa, synthetic construct
ALWYSNHWV

SEQ ID NO: 15 - protein, 125 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 16 - protein, 109 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 17 - protein, 457 aa, synthetic construct
EVQLLESGGGLVQPGGSLRLSCAASGFTFSSYAMNWVRQAPGKGLEWVAGISSSGRYTGYADSVKGRFTISRDNSKNTLYLQMNSLRAEDTAVYYCAKSVGSGVSWSGYVATSLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 211 aa, synthetic construct
SYELTQPPSVSVSPGQTARITCSAGSGLYGWYQQKPGQAPVLVIYGTNKRPSGIPERFSGSSSGTTVTLTISGVQAEDEADYYCGSADSSTNAGIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 19 - protein, 487 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLASPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 20 aa, synthetic construct
GKPGSGKPGSGKPGSGKPGS

SEQ ID NO: 21 - protein, 7 aa, synthetic construct
GFTFSSY

SEQ ID NO: 22 - protein, 6 aa, synthetic construct
SSSGRY

SEQ ID NO: 23 - protein, 8 aa, synthetic construct
GFTFSSYA

SEQ ID NO: 24 - protein, 8 aa, synthetic construct
ISSSGRYT

SEQ ID NO: 25 - protein, 20 aa, synthetic construct
AKSVGSGVSWSGYVATSLDA

SEQ ID NO: 26 - protein, 7 aa, synthetic construct
GFTFSTY

SEQ ID NO: 27 - protein, 8 aa, synthetic construct
RSKYNNYA

SEQ ID NO: 28 - protein, 8 aa, synthetic construct
GFTFSTYA

SEQ ID NO: 29 - protein, 10 aa, synthetic construct
IRSKYNNYAT

SEQ ID NO: 30 - protein, 16 aa, synthetic construct
VRHGNFGDSYVSWFAY

SEQ ID NO: 31 - protein, 9 aa, synthetic construct
TGAVTTSNY

SEQ ID NO: 32 - protein, 254 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 33 - protein, 233 aa, synthetic construct
ASPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

(19) United States
(12) Patent Application Publication
(10) Pub. No.: US 2025/0333499 A1
(43) Pub. Date: Oct. 30, 2025

(54) BISPECIFIC ANTIBODIES AND METHODS OF USING THE SAME

(71) Applicant: Context Therapeutics Inc., Philadelphia, PA (US)

(72) Inventors: Stanley A. Roberts, Northbrook, IL (US); Eric A. Butz, Seattle, WA (US)

(21) Appl. No.: 19/193,290

(22) Filed: Apr. 29, 2025

Related U.S. Application Data
(60) Provisional application No. 63/640,483, filed on Apr. 30, 2024.

(51) Int. Cl. C07K 16/28

(52) U.S. Cl. CPC C07K 16/28; C07K 16/2809; C07K 2317/31; C07K 2317/52; C07K 2317/622

(57) ABSTRACT

Provided for herein are bispecific antibodies comprising a CLDN6 binding domain and a CD3 binding domain, compositions comprising the same and methods of use thereof.

### CROSS-REFERENCE TO RELATED APPLICATIONS

This application claims priority to U.S. Provisional Application Ser. No. 63/640,483, filed Apr. 30, 2024, which is hereby incorporated by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Apr. 29, 2025, is named “CXH-022US_SL” and is 31,511 bytes in size.

### FIELD OF THE DISCLOSURE

The present disclosure is directed generally to bispecific antibodies that bind Claudin 6 (CLDN6) and CD3, compositions comprising the same, and methods of use thereof.

### BACKGROUND

Cell adhesion proteins are critical for maintaining tissue integrity, as well as regulating diverse cellular events in a wide variety of physiological and pathological processes. Among cell adhesion proteins, some members of the claudin (CLDN) family are often aberrantly expressed in various cancers. Clinical application of CLDN therapeutics has been difficult because of lack of antibody specificity for particular CLDN proteins and widespread expression of closely related CLDN family members on normal cells. Thus, there remains a significant need for improved compositions and methods that can modulate the activity of CLDN family members to treat various cancers and diseases.

### BRIEF SUMMARY

In some embodiments, a method of treating a cancer in a subject with a bispecific antibody that binds to Claudin 6 (CLDN6) and CD3 is provided, the method comprising administering a first dose of the bispecific antibody; and administering one or more subsequent doses of the bispecific antibody, wherein the first dose is less than or the same as the one or more subsequent doses.

In some embodiments, the first dose of the bispecific antibody is selected from the group consisting of: about 10 μg to about 1500 μg, about 20 μg to about 1250 μg, about 22.5 μg to about 1000 μg, about 50 μg to about 800 μg, about 70 μg to about 750 μg, about 100 μg to about 500 μg, about 200 μg to about 400 μg, about 500 μg to about 1000 μg, about 600 μg to about 1000 μg, or about 800 μg to about 1000 μg, and the one or more subsequent dose of the bispecific antibody is selected from the group consisting of: about 10 μg to about 15000 μg, about 20 μg to about 13500 μg, about 22.5 μg to about 12800 μg, about 50 μg to about 10000 μg, about 70 μg to about 7500 μg, about 100 μg to about 6400 μg, about 200 μg to about 5000 μg, about 250 μg to about 4000 μg, about 300 μg to about 3200 μg, about 400 μg to about 2500 μg, about 500 μg to about 2000 μg, about 600 μg to about 1600 μg, about 800 μg to about 1000 μg, about 250 μg to about 10000 μg, about 550 μg to about 10000 μg, about 1100 μg to about 10000 μg, about 2000 μg to about 10000 μg, or about 4000 μg to about 10000 μg.

In some embodiments, the bispecific antibody comprises a CLDN6 binding domain and a CD3 binding domain, wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14

In some embodiments, the CLDN6 binding domain comprises a first VH comprising an amino acid sequence having at least 95% identity to the amino acid sequence of SEQ ID NO: 7, and comprises a first VL comprising an amino acid sequence having at least 95% identity to the amino acid sequence of SEQ ID NO: 8.

In some embodiments, the CLDN6 binding domain comprises a HC and a LC, wherein the HC comprises an amino acid sequence having at least 95% identity to the amino acid sequence of SEQ ID NO: 17 and the LC comprises an amino acid sequence having at least 95% identity to the amino acid sequence of SEQ ID NO: 18.

In some embodiments, the CD3 binding domain comprises an amino acid sequence having at least 95% identity to the amino acid sequence of SEQ ID NO: 19.

In some embodiments, the bispecific antibody comprising a CLDN6 binding domain and a CD3 binding domain is a Fab-Fc-scFv.

In some embodiments, the bispecific antibody comprising a CLDN6 binding domain and a CD3 binding domain is an IgG-scFv.

In some embodiments, the bispecific antibody comprising a CLDN6 binding domain and a CD3 binding domain is an IgG-(scFv)₂.

In some embodiments, a method of treating a cancer in a subject in need thereof is provided, the method comprising: intravenously administering to the subject a first dose of a bispecific antibody, wherein the first dose is selected from the group consisting of: about 22.5 μg, about 70 μg, about 140 μg, about 200 μg, about 280 μg, about 400 μg, about 560 μg, about 800 μg, or about 1000 μg, and intravenously administering to the subject one or more subsequent doses, wherein each subsequent dose is selected from the group consisting of: about 22.5 μg, about 70 μg, about 140 μg, about 200 μg, about 280 μg, about 400 μg, about 560 μg, about 800 μg, about 1120 μg, about 1600 μg, about 2240 μg, about 3200 μg, about 4480 μg, about 6400 μg, about 8960 μg, or about 12800 μg, wherein the bispecific antibody comprises a CLDN6 binding domain and a CD3 binding domain; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, the first dose is about 22.5 μg and at least one or more of the one or more subsequent doses is about 22.5 μg. In some embodiments, the first dose is about 22.5 μg and at least one or more of the one or more subsequent doses is about 70 μg. In some embodiments, the first dose is about 70 μg and at least one or more of the one or more subsequent doses is about 140 μg. In some embodiments, the first dose is about 70 μg and at least one or more of the one or more subsequent doses is about 200 μg. In some embodiments, the first dose is about 140 μg and at least one or more of the one or more subsequent doses is about 280 μg. In some embodiments, the first dose is about 200 μg and at least one or more of the one or more subsequent doses is about 400 μg. In some embodiments, the first dose is about 280 μg and at least one or more of the one or more subsequent doses is about 560 μg. In some embodiments, the first dose is about 400 μg and at least one or more of the one or more subsequent doses is about 800 μg. In some embodiments, the first dose is about 560 μg and at least one or more of the one or more subsequent doses is about 1120 μg. In some embodiments, the first dose is about 800 μg and at least one or more of the one or more subsequent doses is about 1600 μg. In some embodiments, the first dose is about 1000 μg and at least one or more of the one or more subsequent doses is about 2240 μg. In some embodiments, the first dose is about 1000 μg and at least one or more of the one or more subsequent doses is about 3200 μg. In some embodiments, the first dose is about 1000 μg and at least one or more of the one or more subsequent doses is about 4480 μg. In some embodiments, the first dose is about 1000 μg and at least one or more of the one or more subsequent doses is about 6400 μg. In some embodiments, the first dose is about 1000 μg and at least one or more of the one or more subsequent doses is about 8960 μg. In some embodiments, the first dose is about 1000 μg and at least one or more of the one or more subsequent doses is about 12800 μg.

In some embodiments, the cancer is a solid tumor. In some embodiments, the solid tumor expresses CLDN6. In some embodiments, the cancer is selected from ovarian cancer, testicular cancer, or endometrial cancer. In some embodiments, the ovarian cancer is platinum-refractory/resistance ovarian cancer (PRROC).

In some embodiments, a method of treating platinum refractory/resistant ovarian cancer (PRROC) in a subject in need thereof is provided, the method comprising intravenously administering to the subject a first dose of a bispecific antibody, wherein the first dose is selected from the group consisting of: about 22.5 μg, about 70 μg, about 140 μg, about 200 μg, about 280 μg, about 400 μg, about 560 μg, about 800 μg, or about 1000 μg, and intravenously administering to the subject one or more subsequent doses, wherein each subsequent dose is selected from the group consisting of: about 22.5 μg, about 70 μg, about 140 μg, about 200 μg, about 280 μg, about 400 μg, about 560 μg, about 800 μg, about 1120 μg, about 1600 μg, about 2240 μg, about 3200 μg, about 4480 μg, about 6400 μg, about 8960 μg, or about 12800 μg, wherein the bispecific antibody comprises a CLDN6 binding domain and a CD3 binding domain; wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

In some embodiments, a method of treating a cancer in a subject in need thereof is provided, the method comprising: intravenously administering to the subject a first dose of a bispecific antibody, wherein the first dose is selected from the group consisting of: about 22.5 μg, about 70 μg, about 140 μg, about 200 μg, about 280 μg, about 400 μg, about 560 μg, about 800 μg, or about 1000 μg, and intravenously administering to the subject one or more subsequent doses, wherein each subsequent dose is selected from the group consisting of: about 22.5 μg, about 70 μg, about 140 μg, about 200 μg, about 280 μg, about 400 μg, about 560 μg, about 800 μg, about 1120 μg, about 1600 μg, about 2240 μg, about 3200 μg, about 4480 μg, about 6400 μg, about 8960 μg, or about 12800 μg, wherein the bispecific antibody binds to claudin 6 (CLDN6) and CD3, the bispecific antibody comprising:
a first polypeptide comprising a first light chain comprising a first variable light chain region, wherein the first variable light chain region comprises:
   (1) a CDR1 sequence comprising the amino acid sequence of SEQ ID NO: 4;
   (2) a CDR2 sequence comprising the amino acid sequence of SEQ ID NO: 5; and
   (3) a CDR3 sequence comprising the amino acid sequence of SEQ ID NO: 6;
a second polypeptide comprising a first heavy chain comprising a first variable heavy chain region and a constant domain, wherein the first variable heavy chain region comprises:
   (1) a CDR1 comprising the amino acid sequence of SEQ ID NO: 1;
   (2) a CDR2 comprising the amino acid sequence of SEQ ID NO: 2, and
   (3) a CDR3 comprising the amino acid sequence of SEQ ID NO: 3; and
a third polypeptide comprising a second light chain and a second heavy chain and a constant domain, wherein:
   the second heavy chain comprises a second variable heavy chain region comprising:
   (1) a CDR1 comprising the amino acid sequence of SEQ ID NO: 9;
   (2) a CDR2 comprising the amino acid sequence of SEQ ID NO: 10; and
   (3) a CDR3 comprising the amino acid sequence of SEQ ID NO: 11; and
   the second light chain comprises a second variable light chain region comprising:
   (1) a CDR1 comprising the amino sequence of SEQ ID NO: 12;
   (2) a CDR2 comprising the amino acid sequence of SEQ ID NO: 13; and
   (3) a CDR3 comprising the amino acid sequence of SEQ ID NO: 14, or a variant thereof,
      wherein: the first polypeptide comprises an amino acid sequence that is at least 95% identical to the amino acid sequence of SEQ ID NO: 18; the second polypeptide comprises an amino acid sequence that is at least 95% identical to the amino acid sequence of SEQ ID NO: 17; and the third polypeptide comprises an amino acid sequence that is at least 95% identical to the amino acid sequence of SEQ ID NO: 19.

### BRIEF DESCRIPTION OF THE DRAWINGS

FIG. 1A-E illustrate exemplary bispecific antibody configurations as provided for herein.

FIG. 1A illustrates a tandem scFv format. FIG. 1B illustrates a Fab-Fc-scFv format. FIG. 1C illustrates an IgG-(scFv)₂ format. FIG. 1D and FIG. 1E illustrate two different IgG-scFv formats.

FIG. 2 illustrates the enrollment criteria for Cohorts 1-3 of a Phase 1a clinical trial.

FIG. 3 illustrates the enrollment criteria for Cohorts 4-9 of a Phase 1a clinical trial.

FIG. 4 illustrates the dose escalation rules for Cohorts 1-3 of a Phase 1a clinical trial.

FIG. 5 illustrates the dose escalation rules for Cohorts 4-9 of a Phase 1a clinical trial.

### DETAILED DESCRIPTION

It is to be understood that the embodiments described herein are not limited to particular formulations, compositions and experimental conditions disclosed, as such formulations, compositions, and experimental conditions may vary. It is also to be understood that the terminology used herein is only for the purpose of describing particular embodiments, and it is not intended to be limiting.

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art.

Unless otherwise required by context, singular terms shall include pluralities, and plural terms shall include the singular.

That the disclosure may be more readily understood, select terms are defined below.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

Additionally, where a phrase recites “about x to y,” the term “about” modifies both x and y and can be used interchangeably with the phrase “about x to about y” unless context dictates differently.

As used herein, the term “individual” or “subject,” or “patient” used interchangeably, means any animal, including mammals, such as mice, rats, other rodents, rabbits, dogs, cats, swine, cattle, sheep, horses, or primates, such as humans.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of” or “consists.”

The term “antibody” as used herein refers to a protein which interacts with an antigen and comprises at least two heavy chains (HC) and two light chains (LC) inter-connected by disulfide bonds. Each heavy chain is comprised of a heavy chain variable region (VH) and a heavy chain constant region. The heavy chain constant region of an IgG is comprised of three domains, CH1, CH2 and CH3. Each light chain is comprised of a light chain variable region (VL) and a light chain constant region. The light chain constant region is comprised of one domain, CL. The VH and VL regions can be further subdivided into hypervariable regions, termed complementarity determining regions (CDR), interspersed with regions that are more conserved, termed framework regions (FR). Each VH and VL is composed of three CDRs and four FRs arranged from N-terminus to C-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes for example, monoclonal antibodies, human antibodies, humanized antibodies, camelized antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

As used herein, the term “antibody fragment” refers to one or more portions of an antibody that retain the ability to specifically interact (e.g., by binding, steric hindrance, stabilizing spatial distribution) with an antigen. Examples of binding fragments include, but are not limited to, a Fab fragment, a monovalent fragment consisting of the VL, VH, CL and CH1 domains; a F(ab′)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment, which consists of a VH domain; and an isolated CDR. Furthermore, although the two domains of the Fv fragment, VL and VH, are coded for by separate genes, they can be joined using recombinant methods by a synthetic linker that allows them to be made as a single protein chain in which the VL and VH regions pair to form monovalent molecules (known as single chain Fv (scFv). Such single chain antibodies are also intended to be encompassed within the term “antibody fragment.” These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR, and bis-scFv. Antibody fragments can be incorporated into single chain molecules comprising a pair of tandem Fv segments (VH-CH1-VH-CH1) which, together with complementary light chain polypeptides, form a pair of antigen-binding sites.

As used herein, the terms “complementarity-determining regions,” “CDRs,” and “hypervariable regions” refer to the parts of the variable domains in antibodies that determine the antibodies' binding specificities to their specific antigen. A single variable region of an antibody polypeptide will typically comprise three CDRs, usually designated CDR1, CDR2, and CDR3. More particularly, a heavy chain variable region may contain CDRs designated HCDR1, HCDR2, and HCDR3; likewise, light chain variable region may contain CDRs LCDR1, LCDR2, and LCDR3. Multiple methods may be used to define a CDR sequence. The current art utilizes various numbering schemes with different definitions of CDR lengths and positions. For example, IMGT numbering scheme is a standardized numbering system based on alignments of sequences from a complete reference gene database including the whole immunoglobulin superfamily. The Kabat numbering scheme is based on sequence alignment and uses “variability parameter” of a given amino acid position (the number of different amino acids at a given position divided by the frequency of the most occurring amino acid at that position) to predict CDRs. The Chothia numbering scheme, on the other hand, is a structure-based numbering scheme where antibody crystal structures are aligned as define the loop structures as CDRs. The Martin numbering scheme focuses on the structure alignment of different framework regions of unconventional lengths. Honneger's numbering scheme (Aho's) is based on structural alignments of the 3D structure of the variable regions and uses structurally conserved Ca positions to deduce framework and CDR lengths. One of skill in the art will note that the definition of a CDR will vary based on the method used. Accordingly, CDR sequences of a given heavy or light chain variable region may vary depending on the numbering system used. Any method of defining a CDR is contemplated with the sequences disclosed herein.

As used herein, the terms “treatment,” “treating,” and the like, in some cases, refer to administering an agent, or carrying out a procedure, for the purposes of obtaining an effect. The effect may be prophylactic in terms of completely or partially preventing a disease or symptom thereof and/or is therapeutic in terms of effecting a partial or complete cure for a disease and/or symptoms of the disease. “Treatment,” as used herein, includes treatment of a disease or disorder in a mammal, particularly in a human, and includes: (a) preventing the disease or a symptom of a disease from occurring in a subject which is predisposed to the disease but has not yet been diagnosed as having it (e.g., including diseases that is associated with or caused by a primary disease; (b) inhibiting the disease, i.e., arresting its development; and (c) relieving the disease, i.e., causing regression of the disease. The term treating includes to any indicia of success in the treatment or amelioration or prevention of a disease or disorder, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters, including the results of an examination by a physician. Accordingly, the term “treating” includes the administration of the agents of the present disclosure to prevent or delay, to alleviate, or to arrest or inhibit development of the symptoms or conditions associated with diseases. The term “therapeutic effect” refers to the reduction, elimination, or prevention of the disease, symptoms of the disease, or side effects of the disease in the subject. A subject is “treated” for a disease or disorder if, after receiving a therapeutic amount of an antibody of the present disclosure, the patient shows observable and/or measurable change in a parameter or symptom of the disease or disorder.

A “Fab fragment” is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

In some embodiments, the antibodies, or antigen fragments herein, comprise a Fc region. In some embodiments, the Fc region comprises a mutation that extends the half-life of the antibody when linked to the Fc region. In some embodiments, the Fc region comprises a S228P, L235E, M252Y, S254T, T256E, M428L, N434S, L234F, P331S mutation, or any combination thereof. In some embodiments, the Fc region comprises a M252Y, S254T, and T256E mutations. In some embodiments, the Fc region comprises a S228P and a L235E mutation. In some embodiments, the antibody comprises a L234F, L235E, and P331S mutation. In some embodiments, the Fc region comprises M252Y, S254T, T256E, S228P and L235E mutations. In some embodiments, the Fc region comprises S228P, L235E, M428L, and N434S mutations. In some embodiments, the Fc region comprises the M428L and N434S mutations. In some embodiments, the Fc region comprises the L234F, L235E, P331S, M252Y, S254T, and T256E mutations. Mutations in the Fc region are also described in US2007041972A1, EP2235059B1, U.S. Pat. No. 8,394,925, and Mueller et al, Mol Immunol 1997 April; 34(6):441-52, each of which is incorporated by reference in its entirety. The numbering referenced herein refers to the Kabat numbering system for the Fc region.

A “Fab′ fragment” contains one light chain and a portion or fragment of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

The term “single-chain Fv” or “scFv” antibody refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific (see below).

In certain embodiments, monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079). In one embodiment, the present invention provides single domain antibodies comprising two V_{H} domains with modifications such that single domain antibodies are formed.

“Isolated antibody” refers to the purification status of a binding compound and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

The term “monoclonal antibody”, as used herein, refers to population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, a “chimeric antibody” is an antibody having the variable domain from a first antibody and constant domain from a second antibody, where the first and second antibodies are from different species. (U.S. Pat. No. 4,816,567; and Morrison et al., (1984) Proc. Natl. Acad. Sci. USA 81: 6851-6855). Typically the variable domains are obtained from an antibody from an experimental animal (the “parental antibody”), such as a rodent, and the constant domain sequences are obtained from human antibodies, so that the resulting chimeric antibody will be less likely to elicit an adverse immune response in a human subject than the parental (e.g. rodent) antibody.

As used herein, the term “humanized antibody” refers to forms of antibodies that contain sequences from both human and non-human (e.g., murine, rat) antibodies. In general, the humanized antibody will comprise substantially all of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc).

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody that comprises mouse immunoglobulin sequences only. Alternatively, a fully human antibody may contain rat carbohydrate chains if produced in a rat, in a rat cell, or in a hybridoma derived from a rat cell. Similarly, “rat antibody” refers to an antibody that comprises rat immunoglobulin sequences only.

Additionally, in some embodiments, the antibodies can take the form of a full length antibody, single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; a Microbody; a peptide aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; Affimers, a DuoBody, a Fv, a Fab, a Fab′, a F(ab′)2, a peptide mimetic molecule, or a synthetic molecule, as described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of each of which are hereby incorporated by reference in their entireties. See also, Storz MAbs. 2011 May-June; 3(3): 310-317, which is hereby incorporated by reference.

The term “antigen” as used herein means any molecule that has the ability to generate antibodies either directly or indirectly or that binds to antibody. Included within the definition of “antigen” is a protein-encoding nucleic acid. An “antigen” can also refer to the binding partner of an antibody. In some embodiments, the antigen is the IGF-1R protein expressed on the surface of a cell. In some embodiments, the cell is an intact cell. An intact cell is a cell that has not been lysed or broken open with the use of detergents or other reagents. A cell that has been treated with detergents or other reagents that breaks up the cellular membrane or punches holes in a cellular membrane is not an intact cell. For example, methods are provided herein for generating an antibody that binds to a IGF-1R protein, the method comprising culturing a cell comprising a nucleic acid molecule encoding the IGF-1R antibody.

As used herein, “specific binding” or “immunospecific binding” or “binds immunospecifically” refer to antibody binding to a predetermined antigen (e.g. IGF-1R) or epitope present on the antigen. In some embodiments, the antibody binds with a dissociation constant (K_{D}) of 10⁻⁷ M or less, and binds to the predetermined antigen with a K_{D} that is at least two-fold less than its K_{D} for binding to a non-specific antigen (e.g., BSA, casein, or another non-specific polypeptide) other than the predetermined antigen. The phrases “an antibody recognizing IGF-1R” and “an antibody specific for IGF-1R” are used interchangeably herein with the term “an antibody which binds immunospecifically to IGF-1R.” Reference in the present disclosure may be made to IGF-1R. The degree of specificity necessary for an anti-IGF-1R antibody may depend on the intended use of the antibody, and at any rate is defined by its suitability for use for an intended purpose. In some embodiments, the antibody, or binding compound derived from the antigen-binding site of an antibody, of the contemplated method binds to its antigen (IGF-1R), with an affinity that is at least two fold greater, at least ten times greater, at least 20-times greater, or at least 100-times greater than the affinity with any other antigen.

Methods for determining mAb specificity and affinity by competitive inhibition can be found in Harlow, et al., Antibodies: A Laboratory Manual, Cold Spring Harbor Laboratory Press, Cold Spring Harbor, N.Y, 1988), Colligan et al., eds., Current Protocols in Immunology, Greene Publishing Assoc. and Wiley Interscience, N.Y, (1992, 1993), and Muller, Meth. Enzymol. 92:589 601 (1983), which references are entirely incorporated herein by reference.

The term “homolog” means protein sequences having between 40% and 100% sequence homology or identity to a reference sequence. Percent identity between two peptide chains can be determined by pair wise alignment using the default settings of the AlignX module of Vector NTI v.9.0.0 (Invitrogen Corp., Carslbad, Calif). In some embodiments, the antibody, or antigenic binding fragment thereof has, at least 50, 60, 70, 80, 90, 91, 92, 93, 94, 95, 96, 97, 98, or 99% homology or identity to a sequence described herein. In some embodiments, the antibody has conservative substitutions as compared to a sequence described herein. Exemplary conservative substitutions are illustrated in Table 1 and are encompassed within the scope of the disclosed subject matter. The conservative substitution may reside in the framework regions, or in antigen-binding sites, as long they do not adversely affect the properties of the antibody. Substitutions may be made to improve antibody properties, for example stability or affinity. Conservative substitutions will produce molecules having functional and chemical characteristics similar to those molecules into which such modifications are made. Exemplary amino acid substitutions are shown in the table below.

TABLE
Exemplary Conservative Substitutions:

| Original Residue | Exemplary Conservative Substitutions |
|---|---|
| Ala | Val, Leu, Ile |
| Arg | Lys, Gln, Asn |
| Asn | Gln |
| Asp | Glu |
| Cys | Ser, Ala |
| Gln | Asn |
| Gly | Pro, Ala |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

In some embodiments, variants of the proteins and peptides provided herein are provided. In some embodiments, a variant comprises a substitution, deletions, or insertion. In some embodiments, the variant comprises 1, 2, 3, 4, 5, 6, 7, 8, 9, or 10 (e.g., 1-10) substitutions. As described herein, the substitutions can be conservative substitutions. In some embodiments, the substitution is non-conservative. In some embodiments, the variant comprises 1, 2, 3, 4, 5, 6, 7, 8, 9, or 10 (e.g., 1-10) deletions. In some embodiments, the variant comprises 1, 2, 3, 4, 5, 6, 7, 8, 9, or 10 (e.g., 1-10) insertions. In some embodiments, the substitutions, deletions, or insertions are present in the CDRs provided for herein. In some embodiments, the substitutions, deletions, or insertions are not present in the CDRs provided for herein.

The term “in combination with” as used herein means that the described agents can be administered to an animal or subject together in a mixture, concurrently as single agents or sequentially as single agents in any order.

The techniques to raise antibodies to small peptide sequences that recognize and bind to those sequences in the free or conjugated form or when presented as a native sequence in the context of a large protein are well known in the art. Such antibodies include murine, murine-human and human-human antibodies produced by hybridoma or recombinant techniques known in the art. Antibodies can also be produced in human, a mouse, sheep, a rat, a rabbit, a shark, a llama, or a chicken. In some embodiments, the antibody is produced in a chicken. The antibodies can also be produced in or other small animals.

The term “epitope” is meant to refer to that portion of any molecule capable of being recognized by and bound by an antibody at one or more of the Ab's antigen binding regions. Epitopes usually consist of chemically active surface groupings of molecules such as amino acids or sugar side chains and have specific three dimensional structural characteristics as well as specific charge characteristics. Example of epitopes include, but are not limited to, the residues described herein that form IGF-1R epitopes. In some embodiments, the epitope is only present in a non-denatured protein. In some embodiments, the epitope is only present in a denatured protein.

### Bispecific Antibodies

In some embodiments, a bispecific antibody is provided, the bispecific antibody comprising a claudin 6 (CLDN6) binding domain and a CD3 binding domain. In some embodiments, the format of the bispecific antibody is as provided for herein. In some embodiments, the format of the bispecific antibody is a tandem scFv antibody, a Fab-Fc-scFv antibody, an IgG-(scFv)₂ antibody, or an IgG-(scFv)₁ antibody. In some embodiments, the bispecific antibody comprises a CLDN6 binding domain and a CD3 binding domain and is a tandem scFv antibody. In some embodiments, the bispecific antibody comprises a CLDN6 binding domain and a CD3 binding domain and is a tandem Fab-Fc-scFv antibody. In some embodiments, the bispecific antibody comprises a CLDN6 binding domain and a CD3 binding domain and is an IgG-(scFv)₂ antibody. In some embodiments, the bispecific antibody comprises a CLDN6 binding domain and a CD3 binding domain and is an IgG-(scFv)₁ antibody.

In some embodiments, the bispecific antibody comprises a CLDN6 binding domain and a CD3 binding domain, wherein the CLDN6 binding domain comprises a first variable heavy chain region (V_{H}) and a first variable light chain region (V_{L}) and wherein the CD3 binding domain comprises a second variable heavy chain region (V_{H}) and a second variable light chain region (V_{L}). In some embodiments, the bispecific antibody comprises a CLDN6 binding domain and a CD3 binding domain, wherein the CLDN6 binding domain comprises a first V_{H} comprising a HCDR1, HCDR2, and HCDR3 as provided for in Tables 1, 2, or 3, and a first V_{L} comprising a LCDR1, LCDR2, and LCDR3 as provided for in Tables 1, 2, or 3; and wherein the CD3 binding domain comprises a second V_{H} comprising a HCDR1, HCDR2, and HCDR3 as provided for in Tables 4, 5, or 6 and a second V_{L} comprising a LCDR1, LCDR2, and LCDR3 as provided for in Tables 4, 5, or 6.

TABLE 1
CDRs of CLDN6 binding domain - Kabat numbering
Kabat CDRs

| Domain | HCDR1 | HCDR2 | HCDR3 | LCDR1 | LCDR2 | LCDR3 |
|---|---|---|---|---|---|---|
| CLDN6 | SYAMN | GISSSGRYT | SVGSGVSWS | SAGSGLY | GTNKRPS | GSADSST |
| binding | (SEQ | GYADSVKG | GYVATSLDA | G (SEQ | (SEQ ID | NAGI |
| domain | ID NO: | (SEQ ID | (SEQ ID | ID NO: | NO: 5) | (SEQ ID |
| | 1) | NO: 2) | NO: 3) | 4) | | NO: 6) |

TABLE 2
CDRs of CLDN6 binding domain - Chothia numbering
Chothia CDRs

| Domain | HCDR1 | HCDR2 | HCDR3 | LCDR1 | LCDR2 | LCDR3 |
|---|---|---|---|---|---|---|
| CLDN6 | GFTFSSY | SSSGRY | SVGSGVSWS | SAGSGLY | GTNKRPS | GSADSSTNA |
| binding | (SEQ ID | (SEQ | GYVATSLDA | G (SEQ | (SEQ ID | GI |
| domain | NO: 21) | ID NO: | (SEQ ID | ID NO: | NO: 5) | (SEQ ID |
| | | 22) | NO: 3) | 4) | | NO: 6) |

TABLE 3
CDRs of CLDN6 binding domain - IMGT numbering
IMGT CDRs

| Domain | HCDR1 | HCDR2 | HCDR3 | LCDR1 | LCDR2 | LCDR3 |
|---|---|---|---|---|---|---|
| CLDN6 | GFTFSSYA | ISSSGRYT | AKSVGSG | SGL | GT | GSADSST |
| binding | (SEQ ID | (SEQ ID | VSWSGYV | (tripep- | (dipep- | NAGI |
| domain | NO: 23) | NO: 24) | ATSLDA | tide) | tide) | (SEQ ID |
| | | | (SEQ ID | | | NO: 6) |
| | | | NO: 25) | | | |

TABLE 4
CDRs of CD3 binding domain - Kabat numbering
Kabat CDRs

| Domain | HCDR1 | HCDR2 | HCDR3 | LCDR1 | LCDR2 | LCDR3 |
|---|---|---|---|---|---|---|
| CD3 | TYAMN | RIRSKYN | HGNFGDSYV | GSSTGAV | GTNKRAP | ALWYSNHWV |
| binding | (SEQ ID | NYATYYA | SWFAY | TTSNYAN | (SEQ ID | (SEQ ID |
| domain | NO: 9) | DSVKG | (SEQ ID | (SEQ ID | NO: 13) | NO: 14) |
| | | (SEQ ID | NO: 11) | NO: 12) | | |
| | | NO: 10) | | | | |

TABLE 5
CDRs of CD3 binding domain - Chothia numbering
Chothia CDRs

| Domain | HCDR1 | HCDR2 | HCDR3 | LCDR1 | LCDR2 | LCDR3 |
|---|---|---|---|---|---|---|
| CD3 | GFTFSTY | RSKYNNY | HGNFGDSYV | GSSTGAV | GTNKRAP | ALWYSNHWV |
| binding | (SEQ ID | A (SEQ | SWFAY | TTSNYAN | (SEQ ID | (SEQ ID |
| domain | NO: 26) | ID NO: | (SEQ ID | (SEQ ID | NO: 13) | NO: 14) |
| | | 27) | NO: 11) | NO: 12) | | |

TABLE 6
CDRs of CD3 binding domain - IMGT numbering
IMGT CDRs

| Domain | HCDR1 | HCDR2 | HCDR3 | LCDR1 | LCDR2 | LCDR3 |
|---|---|---|---|---|---|---|
| CD3 | GFTFSTYA | IRSKYNN | VRHGNFGDS | TGAVTTS | GT | ALWYSNH |
| binding | (SEQ ID | YAT | YVSWFAY | NY (SEQ | (dipep- | WV (SEQ |
| domain | NO: 28) | (SEQ ID | (SEQ ID | ID NO: | tide) | ID NO: |
| | | NO: 29) | NO: 30) | 31) | | 14) |

In some embodiments, the bispecific antibody comprises a CLDN6 binding domain and a CD3 binding domain, wherein the CLDN6 binding domain comprises (i) a first V_{H} comprising heavy chain CDR1, CDR2, and CDR3 sequences, wherein the heavy chain CDR1 sequence has the amino acid sequence of SEQ ID NO: 1; the heavy chain CDR2 sequence has the amino acid sequence of SEQ ID NO: 2; and the heavy chain CDR3 sequence has the amino acid sequence of SEQ ID NO: 3; and (ii) a first V_{L} comprising light chain CDR1, CDR2, and CDR3 sequences, wherein the light chain CDR1 sequence has the amino acid sequence of SEQ ID NO: 4; the light chain CDR2 sequence has the amino acid sequence of SEQ ID NO: 5; and the light chain CDR3 sequence has the amino acid sequence of SEQ ID NO: 6; and wherein the CD3 binding domain comprises (i) a second V_{H} comprising heavy chain CDR1, CDR2, and CDR3 sequences, wherein the heavy chain CDR1 sequence has the amino acid sequence of SEQ ID NO: 9; the heavy chain CDR2 sequence has the amino acid sequence of SEQ ID NO: 10; and the heavy chain CDR3 sequence has the amino acid sequence of SEQ ID NO: 11; and (ii) a second V_{L} comprising light chain CDR1, CDR2, and CDR3 sequences, wherein the light chain CDR1 sequence has the amino acid sequence of SEQ ID NO: 12; the light chain CDR2 sequence has the amino acid sequence of SEQ ID NO: 13; and the light chain CDR3 sequence has the amino acid sequence of SEQ ID NO: 14.

In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises an amino acid sequence as provided for in Table 7, (ii) the first V_{L} comprises an amino acid sequence as provided for in Table 7, (iii) the second V_{H} comprises an amino acid sequence as provided for in Table 8, and (iv) the second V_{L} comprises an amino acid sequence as provided for in Table 8.

TABLE 7
Sequences of the VH region and VL region of the
CLDN6 binding domain

| Domain | SEQ ID NO | Sequence |
|---|---|---|
| V_{H} - CLDN6 | SEQ ID NO: | EVQLLESGGGLVQPGGSLRLSCAASGFTFSSYAMNWVRQAPGKG |
| binding | 7 | LEWVAGISSSGRYTGYADSVKGRFTISRDNSKNTLYLQMNSLRA |
| domain | | EDTAVYYCAKSVGSGVSWSGYVATSLDAWGQGTLVTVSS |
| V_{L} - CLDN6 | SEQ ID NO: | SYELTQPPSVSVSPGQTARITCSAGSGLYGWYQQKPGQAPVLVI |
| binding | 8 | YGTNKRPSGIPERFSGSSSGTTVTLTISGVQAEDEADYYCGSAD |
| domain | | SSTNAGIFGGGTKLTVL |

TABLE 8
Sequences of the VH region and VL region of
the CD3 binding domain

| Domain | SEQ ID NO | Sequence |
|---|---|---|
| VH - CD3 | SEQ ID NO: | EVQLVESGGGLVQPGGSLRLSCAASGFTFSTYAMNWVRQAPG |
| binding | 15 | KGLEWVGRIRSKYNNYATYYADSVKGRFTISRDDSKNTLYLQ |
| domain | | MNSLRAEDTAVYYCVRHGNFGDSYVSWFAYWGQGTLVTVSS |
| VL - CD3 | SEQ ID NO: | QAVVTQEPSLTVSPGGTVTLTCGSSTGAVTTSNYANWVQQKP |
| binding | 16 | GKSPRGLIGGTNKRAPGVPARFSGSLLGGKAALTISGAQPED |
| domain | | EADYYCALWYSNHWVFGGGTKLTVL |

In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, (iii) the second V_{H} comprises an amino acid sequence having at least 90%, 91%, 92%, 93%, 94%, 95%, 96%, 97%, 98%, or 99% identity to the amino acid sequence of SEQ ID NO: 15 as provided for herein, and (iv) the second V_{L} comprises an amino acid sequence having at least 90%, 91%, 92%, 93%, 94%, 95%, 96%, 97%, 98%, or 99% identity to the amino acid sequence of SEQ ID NO: 16 as provided for herein.

In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises an amino acid sequence having at least 95% identity to the amino acid sequence of SEQ ID NO: 7, (ii) the first V_{L} comprises an amino acid sequence having at least 95% identity to the amino acid sequence of SEQ ID NO: 8, (iii) the second V_{H} comprises an amino acid sequence having at least 95% identity to the amino acid sequence of SEQ ID NO: 15, and (iv) the second V_{L} comprises an amino acid sequence having at least 95% identity to the amino acid sequence of SEQ ID NO: 16. In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises an amino acid sequence having at least 98% identity to the amino acid sequence of SEQ ID NO: 7, (ii) the first V_{L} comprises an amino acid sequence having at least 98% identity to the amino acid sequence of SEQ ID NO: 8, (iii) the second V_{H} comprises an amino acid sequence having at least 98% identity to the amino acid sequence of SEQ ID NO: 15, and (iv) the second V_{L} comprises an amino acid sequence having at least 98% identity to the amino acid sequence of SEQ ID NO: 16. In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises the amino acid sequence of SEQ ID NO: 7, (ii) the first V_{L} comprises the amino acid sequence of SEQ ID NO: 8, (iii) the second V_{H} comprises the amino acid sequence of SEQ ID NO: 15, and (iv) the second V_{L} comprises the amino acid sequence of SEQ ID NO: 16.

In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7, provided that the first V_{H} comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3, (ii) the first V_{L} comprises an amino acid sequence having at least 90%, 91%, 92%, 93%, 94%, 95%, 96%, 97%, 98%, or 99% identity to the amino acid sequence of SEQ ID NO: 8, provided that the first V_{L} comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, (iii) the second V_{H} comprises an amino acid sequence having at least 90%, 91%, 92%, 93%, 94%, 95%, 96%, 97%, 98%, or 99% identity to the amino acid sequence of SEQ ID NO: 15, provided that the second V_{H} comprises a HCDR1 comprising the amino acid sequence of SEQ ID NO: 9, a HCDR2 comprising the amino acid sequence of SEQ ID NO: 10, and a HCDR3 comprising the amino acid sequence of SEQ ID NO: 11, and (iv) the second V_{L} comprises an amino acid sequence having at least 90%, 91%, 92%, 93%, 94%, 95%, 96%, 97%, 98%, or 99% identity to the amino acid sequence of SEQ ID NO: 16, provided that the second V_{L} comprises a LCDR1 comprising the amino acid sequence of SEQ ID NO: 12, a LCDR2 comprising the amino acid sequence of SEQ ID NO: 13, and a LCDR3 comprising the amino acid sequence of SEQ ID NO: 14.

In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises an amino acid sequence having at least 95% identity to the amino acid sequence of SEQ ID NO: 7, provided that the first V_{H} comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3, (ii) the first V_{L} comprises an amino acid sequence having at least 95% identity to the amino acid sequence of SEQ ID NO: 8, provided that the first V_{L} comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, (iii) the second V_{H} comprises an amino acid sequence having at least 95% identity to the amino acid sequence of SEQ ID NO: 15, provided that the second V_{H} comprises a HCDR1 comprising the amino acid sequence of SEQ ID NO: 9, a HCDR2 comprising the amino acid sequence of SEQ ID NO: 10, and a HCDR3 comprising the amino acid sequence of SEQ ID NO: 11, and (iv) the second V_{L} comprises an amino acid sequence having at least 95% identity to the amino acid sequence of SEQ ID NO: 16, provided that the second V_{L} comprises a LCDR1 comprising the amino acid sequence of SEQ ID NO: 12, a LCDR2 comprising the amino acid sequence of SEQ ID NO: 13, and a LCDR3 comprising the amino acid sequence of SEQ ID NO: 14.

In some embodiments, the bispecific antibody comprises a CLDN6 binding domain and a CD3 binding domain, wherein the CLDN6 binding domain comprises a first V_{H} and a first V_{L} and wherein the CD3 binding domain comprises a second V_{H} and a second V_{L}, wherein (i) the first V_{H} comprises an amino acid sequence having at least 98% identity to the amino acid sequence of SEQ ID NO: 7, provided that the first V_{H} comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3, (ii) the first V_{L} comprises an amino acid sequence having at least 98% identity to the amino acid sequence of SEQ ID NO: 8, provided that the first V_{L} comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, (iii) the second V_{H} comprises an amino acid sequence having at least 98% identity to the amino acid sequence of SEQ ID NO: 15, provided that the second V_{H} comprises a HCDR1 comprising the amino acid sequence of SEQ ID NO: 9, a HCDR2 comprising the amino acid sequence of SEQ ID NO: 10, and a HCDR3 comprising the amino acid sequence of SEQ ID NO: 11, and (iv) the second V_{L} comprises an amino acid sequence having at least 98% identity to the amino acid sequence of SEQ ID NO: 16, provided that the second V_{L} comprises a LCDR1 comprising the amino acid sequence of SEQ ID NO: 12, a LCDR2 comprising the amino acid sequence of SEQ ID NO: 13, and a LCDR3 comprising the amino acid sequence of SEQ ID NO: 14.

In some embodiments, the bispecific antibody comprises a CLDN6 binding domain and a CD3 binding domain, wherein the CLDN6 binding domain comprises a heavy chain (HC) and a light chain (LC), the heavy chain comprising a first V_{H} as provided for in Table 7 and the LC comprising a first V_{L} as provided for in Table 7, and wherein the CD3 binding domain comprises a scFv comprising a second V_{H} as provided for in Table 8 and a second V_{L} as provided for in Table 8.

In some embodiments, the bispecific antibody comprises a CLDN6 binding domain and a CD3 binding domain, wherein the CLDN6 binding domain comprises a HC comprising an amino acid sequence as provided for in Table 9 and a LC comprising an amino acid sequence as provided for in Table 9, and wherein the CD3 binding domain comprises a scFv comprising an amino acid sequence as provided for in Table 10.

TABLE 9
Sequences of the HC and LC of the CLDN6 binding domain

| Domain | SEQ ID NO | Sequence |
|---|---|---|
| HC - CLDN6 | SEQ ID NO: | EVQLLESGGGLVQPGGSLRLSCAASGFTFSSYAMNWVRQAPG |
| | 17 | KGLEWVAGISSSGRYTGYADSVKGRFTISRDNSKNTLYLQMN |
| | | SLRAEDTAVYYCAKSVGSGVSWSGYVATSLDAWGQGTLVTVS |
| | | SASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPEAAGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLWCL |
| | | VKGFYPSDIAVEWESNGQPENNYKTTPPVLDSDGSFFLYSKL |
| | | TVDKSRWQQGNVFSCSVMHEALHNHYTQKSLSLSPGK |
| LC - CLDN6 | SEQ ID NO: | SYELTQPPSVSVSPGQTARITCSAGSGLYGWYQQKPGQAPVL |
| | 18 | VIYGTNKRPSGIPERFSGSSSGTTVTLTISGVQAEDEADYYC |
| | | GSADSSTNAGIFGGGTKLTVLGQPKAAPSVTLFPPSSEELQA |
| | | NKATLVCLISDFYPGAVTVAWKADSSPVKAGVETTTPSKQSN |
| | | NKYAASSYLSLTPEQWKSHRSYSCQVTHEGSTVEKTVAPTEC |
| | | S |

TABLE 10
Sequence of CD3 binding domain in scFv format
without a constant domain.

| Domain | SEQ ID NO | Sequence |
|---|---|---|
| CD3 binding | SEQ ID NO: | EVQLVESGGGLVQPGGSLRLSCAASGFTFSTYAMNWVRQAPG |
| domain scFv | 32 | KGLEWVGRIRSKYNNYATYYADSVKGRFTISRDDSKNTLYLQ |
| | | MNSLRAEDTAVYYCVRHGNFGDSYVSWFAYWGQGTLVTVSSG |
| | | KPGSGKPGSGKPGSGKPGSQAVVTQEPSLTVSPGGTVTLTCG |
| | | SSTGAVTTSNYANWVQQKPGKSPRGLIGGTNKRAPGVPARFS |
| | | GSLLGGKAALTISGAQPEDEADYYCALWYSNHWVFGGGTKLT |
| | | VL |

In some embodiments, the bispecific antibody comprises a CLDN6 binding domain and a CD3 binding domain, wherein the CLDN6 binding domain comprises a HC comprising an amino acid sequence having at least 90%, 91%, 92%, 93%, 94%, 95%, 96%, 97%, 98%, or 99% identity to the amino acid sequence of SEQ ID NO: 17 and a LC comprising an amino acid sequence having at least 90%, 91%, 92%, 93%, 94%, 95%, 96%, 97%, 98%, or 99% identity to the amino acid sequence of SEQ ID NO: 18, and wherein the CD3 binding domain comprises a scFv comprising an amino acid sequence having at least 90%, 91%, 92%, 93%, 94%, 95%, 96%, 97%, 98%, or 99% identity to the amino acid sequence of SEQ ID NO: 32.

In some embodiments, the bispecific antibody comprises a CLDN6 binding domain comprising a HC and a LC and a CD3 binding domain comprising an scFv, wherein (i) the HC comprises an amino acid sequence having at least 95% identity to the amino acid sequence of SEQ ID NO: 17, (ii) the LC comprises an amino acid sequence having at least 95% identity to the amino acid sequence of SEQ ID NO: 18, and (iii) the scFv comprises an amino acid sequence having at least 95% identity to the amino acid sequence of SEQ ID NO: 32. In some embodiments, the bispecific antibody comprises a CLDN6 binding domain comprising a HC and a LC and a CD3 binding domain comprising an scFv, wherein (i) the HC comprises an amino acid sequence having at least 98% identity to the amino acid sequence of SEQ ID NO: 17, (ii) the LC comprises an amino acid sequence having at least 98% identity to the amino acid sequence of SEQ ID NO: 18, and (iii) the scFv comprises an amino acid sequence having at least 98% identity to the amino acid sequence of SEQ ID NO: 32. In some embodiments, the bispecific antibody comprises a CLDN6 binding domain comprising a HC and a LC and a CD3 binding domain comprising an scFv, wherein (i) the HC comprises the amino acid sequence of SEQ ID NO: 17, (ii) the LC comprises the amino acid sequence of SEQ ID NO: 18, and (iii) the scFv comprises the amino acid sequence of SEQ ID NO: 32.

In some embodiments, the V_{H} and the V_{L} of the scFv are linked directly to one another. In some embodiments, the V_{H} and the V_{L} are linked indirectly to one another, such as through a polypeptide linker. Polypeptide linkers are known in the art and any such polypeptide linker is within the scope of the present disclosure. In some embodiments, the polypeptide linker comprises the amino acid sequence of GKPGSGKPGSGKPGSGKPGS (SEQ ID NO: 20).

In some embodiments, the bispecific antibody comprises a CLDN6 binding domain and a CD3 binding domain, wherein the CLDN6 binding domain comprises a heavy chain (HC) and a light chain (LC), the heavy chain comprising a first V_{H} comprising an amino acid sequence as provided for in Table 7 and the LC comprising a first V_{L} comprising an amino acid sequence as provided for Table 7, and wherein the CD3 binding domain comprises a scFv comprising a second V_{H} comprising an amino acid sequence as provided for in Table 8 and a second V_{L} comprising an amino acid sequence as provided for in Table 8, and wherein the CD3 binding domain further comprises a constant domain, wherein the CD3 binding domain comprises an amino acid sequence as provided for in Table 11.

TABLE 11
Sequences of the scFv and constant region of an
exemplary CD3 binding domain

| Domain | SEQ ID NO | Sequence |
|---|---|---|
| CD3 binding | SEQ ID NO: | EVQLVESGGGLVQPGGSLRLSCAASGFTFSTYAMNWVRQAPG |
| domain scFv + | 19 | KGLEWVGRIRSKYNNYATYYADSVKGRFTISRDDSKNTLYLQ |
| constant | | MNSLRAEDTAVYYCVRHGNFGDSYVSWFAYWGQGTLVTVSSG |
| | | KPGSGKPGSGKPGSGKPGSQAVVTQEPSLTVSPGGTVTLTCG |
| | | SSTGAVTTSNYANWVQQKPGKSPRGLIGGTNKRAPGVPARFS |
| | | GSLLGGKAALTISGAQPEDEADYYCALWYSNHWVFGGGTKLT |
| | | VLASPKSSDKTHTCPPCPAPEAAGGPSVFLFPPKPKDTLMIS |
| | | RTPEVTCVVVDVSHEDPEVKFNWYVDGVEVHNAKTKPREEQY |
| | | NSTYRVVSVLTVLHQDWLNGKEYKCKVSNKALPAPIEKTISK |
| | | AKGQPREPQVYTLPPSREEMTKNQVSLSCAVKGFYPSDIAVE |
| | | WESNGQPENNYKTTPPVLDSDGSFFLVSKLTVDKSRWQQGNV |
| | | FSCSVMHEALHNHYTQKSLSLSPGK |

In some embodiments, the bispecific antibody comprises a CLDN6 binding domain and a CD3 binding domain, wherein the CLDN6 binding domain comprises a HC comprising an amino acid sequence as provided for in Table 9 and a LC comprising an amino acid sequence as provided for in Table 9, and wherein the CD3 binding domain comprises a scFv and a constant domain comprising an amino acid sequence as provided for in Table 11.

In some embodiments, the bispecific antibody comprises a CLDN6 binding domain and a CD3 binding domain, wherein the CLDN6 binding domain comprises a HC comprising an amino acid sequence having at least 90%, 91%, 92%, 93%, 94%, 95%, 96%, 97%, 98%, or 99% identity to the amino acid sequence of SEQ ID NO: 17 and a LC comprising an amino acid sequence having at least 90%, 91%, 92%, 93%, 94%, 95%, 96%, 97%, 98%, or 99% identity to the amino acid sequence of SEQ ID NO: 18, and wherein the CD3 binding domain comprises a scFv and a constant domain and comprises an amino acid sequence having at least 90%, 91%, 92%, 93%, 94%, 95%, 96%, 97%, 98%, or 99% identity to the amino acid sequence of SEQ ID NO: 19.

In some embodiments, the bispecific antibody comprises a CLDN6 binding domain comprising a HC and a LC and a CD3 binding domain comprising an scFv and constant domain, wherein (i) the HC comprises an amino acid sequence having at least 95% identity to the amino acid sequence of SEQ ID NO: 17, (ii) the LC comprises an amino acid sequence having at least 95% identity to the amino acid sequence of SEQ ID NO: 18, and (iii) the scFv and constant domain comprises an amino acid sequence having at least 95% identity to the amino acid sequence of SEQ ID NO: 19. In some embodiments, the bispecific antibody comprises a CLDN6 binding domain comprising a HC and a LC and a CD3 binding domain comprising an scFv and constant domain, wherein (i) the HC comprises an amino acid sequence having at least 98% identity to the amino acid sequence of SEQ ID NO: 17, (ii) the LC comprises an amino acid sequence having at least 98% identity to the amino acid sequence of SEQ ID NO: 18, and (iii) the scFv and constant domain comprises an amino acid sequence having at least 98% identity to the amino acid sequence of SEQ ID NO: 19. In some embodiments, the bispecific antibody comprises a CLDN6 binding domain comprising a HC and a LC and a CD3 binding domain comprising an scFv and constant domain, wherein (i) the HC comprises the amino acid sequence of SEQ ID NO: 17, (ii) the LC comprises the amino acid sequence of SEQ ID NO: 18, and (iii) the scFv and constant domain comprises the amino acid sequence of SEQ TD NO: 19. In some non-limiting embodiments, the bispecific antibody comprising a CLDN6 binding domain and a CD3 binding domain comprises one or more sequences as provided in Table 12 below:

TABLE 12
Exemplary bispecific antibody sequences

| Binding | | | |
|---|---|---|---|
| Domain | Region | SEQ ID NO | Sequence |
| CLDN6 | HCDR1 | SEQ ID NO: 1 | SYAMN |
| binding | | | |
| domain | | | |
| | HCDR2 | SEQ ID NO: 2 | GISSSGRYTGYADSVKG |
| | HCDR3 | SEQ ID NO: 3 | SVGSGVSWSGYVATSLDA |
| | LCDR1 | SEQ ID NO: 4 | SAGSGLYG |
| | LCDR2 | SEQ ID NO: 5 | GTNKRPS |
| | LCDR3 | SEQ ID NO: 6 | GSADSSTNAGI |
| | VH | SEQ ID NO: 7 | EVQLLESGGGLVQPGGSLRLSCAASGFTFSS |
| | | | YAMNWVRQAPGKGLEWVAGISSSGRYTGYAD |
| | | | SVKGRFTISRDNSKNTLYLQMNSLRAEDTAV |
| | | | YYCAKSVGSGVSWSGYVATSLDAWGQGTLVT |
| | | | VSS |
| | VL | SEQ ID NO: 8 | SYELTQPPSVSVSPGQTARITCSAGSGLYGW |
| | | | YQQKPGQAPVLVIYGTNKRPSGIPERFSGSS |
| | | | SGTTVTLTISGVQAEDEADYYCGSADSSTNA |
| | | | GIFGGGTKLTVL |
| | HC | SEQ ID NO: 17 | EVQLLESGGGLVQPGGSLRLSCAASGFTFSS |
| | | | YAMNWVRQAPGKGLEWVAGISSSGRYTGYAD |
| | | | SVKGRFTISRDNSKNTLYLQMNSLRAEDTAV |
| | | | YYCAKSVGSGVSWSGYVATSLDAWGQGTLVT |
| | | | VSSASTKGPSVFPLAPSSKSTSGGTAALGCL |
| | | | VKDYFPEPVTVSWNSGALTSGVHTFPAVLQS |
| | | | SGLYSLSSVVTVPSSSLGTQTYICNVNHKPS |
| | | | NTKVDKKVEPKSCDKTHTCPPCPAPEAAGGP |
| | | | SVFLFPPKPKDTLMISRTPEVTCVVVDVSHE |
| | | | DPEVKFNWYVDGVEVHNAKTKPREEQYNSTY |
| | | | RVVSVLTVLHQDWLNGKEYKCKVSNKALPAP |
| | | | IEKTISKAKGQPREPQVYTLPPSREEMTKNQ |
| | | | VSLWCLVKGFYPSDIAVEWESNGQPENNYKT |
| | | | TPPVLDSDGSFFLYSKLTVDKSRWQQGNVFS |
| | | | CSVMHEALHNHYTQKSLSLSPGK |
| | LC | SEQ ID NO: 18 | SYELTQPPSVSVSPGQTARITCSAGSGLYGW |
| | | | YQQKPGQAPVLVIYGTNKRPSGIPERFSGSS |
| | | | SGTTVTLTISGVQAEDEADYYCGSADSSTNA |
| | | | GIFGGGTKLTVLGQPKAAPSVTLFPPSSEEL |
| | | | QANKATLVCLISDFYPGAVTVAWKADSSPVK |
| | | | AGVETTTPSKQSNNKYAASSYLSLTPEQWKS |
| | | | HRSYSCQVTHEGSTVEKTVAPTECS |
| CD3 | HCDR1 | SEQ ID NO: 9 | TYAMN |
| binding | | | |
| domain | | | |
| | HCDR2 | SEQ ID NO: 10 | RIRSKYNNYATYYADSVKG |
| | HCDR3 | SEQ ID NO: 11 | HGNFGDSYVSWFAY |
| | LCDR1 | SEQ ID NO: 12 | GSSTGAVTTSNYAN |
| | LCDR2 | SEQ ID NO: 13 | GTNKRAP |
| | LCDR3 | SEQ ID NO: 14 | ALWYSNHWV |
| | VH | SEQ ID NO: 15 | EVQLVESGGGLVQPGGSLRLSCAASGFTFST |
| | | | YAMNWVRQAPGKGLEWVGRIRSKYNNYATYY |
| | | | ADSVKGRFTISRDDSKNTLYLQMNSLRAEDT |
| | | | AVYYCVRHGNFGDSYVSWFAYWGQGTLVTVS |
| | | | S |
| | VL | SEQ ID NO: 16 | QAVVTQEPSLTVSPGGTVTLTCGSSTGAVTT |
| | | | SNYANWVQQKPGKSPRGLIGGTNKRAPGVPA |
| | | | RFSGSLLGGKAALTISGAQPEDEADYYCALW |
| | | | YSNHWVFGGGTKLTVL |
| | scFv | SEQ ID NO: 32 | EVQLVESGGGLVQPGGSLRLSCAASGFTFST |
| | | | YAMNWVRQAPGKGLEWVGRIRSKYNNYATYY |
| | | | ADSVKGRFTISRDDSKNTLYLQMNSLRAEDT |
| | | | AVYYCVRHGNFGDSYVSWFAYWGQGTLVTVS |
| | | | SGKPGSGKPGSGKPGSGKPGSQAVVTQEPSL |
| | | | TVSPGGTVTLTCGSSTGAVTTSNYANWVQQK |
| | | | PGKSPRGLIGGTNKRAPGVPARFSGSLLGGK |
| | | | AALTISGAQPEDEADYYCALWYSNHWVFGGG |
| | | | TKLTVL |
| | Constant | SEQ ID NO: 33 | ASPKSSDKTHTCPPCPAPEAAGGPSVFLFPP |
| | domain | | KPKDTLMISRTPEVTCVVVDVSHEDPEVKFN |
| | | | WYVDGVEVHNAKTKPREEQYNSTYRVVSVLT |
| | | | VLHQDWLNGKEYKCKVSNKALPAPIEKTISK |
| | | | AKGQPREPQVYTLPPSREEMTKNQVSLSCAV |
| | | | KGFYPSDIAVEWESNGQPENNYKTTPPVLDS |
| | | | DGSFFLVSKLTVDKSRWQQGNVFSCSVMHEA |
| | | | LHNHYTQKSLSLSPGK |
| | scFv + | SEQ ID NO: 19 | EVQLVESGGGLVQPGGSLRLSCAASGFTFST |
| | constant | | YAMNWVRQAPGKGLEWVGRIRSKYNNYATYY |
| | domain | | ADSVKGRFTISRDDSKNTLYLQMNSLRAEDT |
| | | | AVYYCVRHGNFGDSYVSWFAYWGQGTLVTVS |
| | | | SGKPGSGKPGSGKPGSGKPGSQAVVTQEPSL |
| | | | TVSPGGTVTLTCGSSTGAVTTSNYANWVQQK |
| | | | PGKSPRGLIGGTNKRAPGVPARFSGSLLGGK |
| | | | AALTISGAQPEDEADYYCALWYSNHWVFGGG |
| | | | TKLTVLASPKSSDKTHTCPPCPAPEAAGGPS |
| | | | VFLFPPKPKDTLMISRTPEVTCVVVDVSHED |
| | | | PEVKFNWYVDGVEVHNAKTKPREEQYNSTYR |
| | | | VVSVLTVLHQDWLNGKEYKCKVSNKALPAPI |
| | | | EKTISKAKGQPREPQVYTLPPSREEMTKNQV |
| | | | SLSCAVKGFYPSDIAVEWESNGQPENNYKTT |
| | | | PPVLDSDGSFFLVSKLTVDKSRWQQGNVFSC |
| | | | SVMHEALHNHYTQKSLSLSPGK |

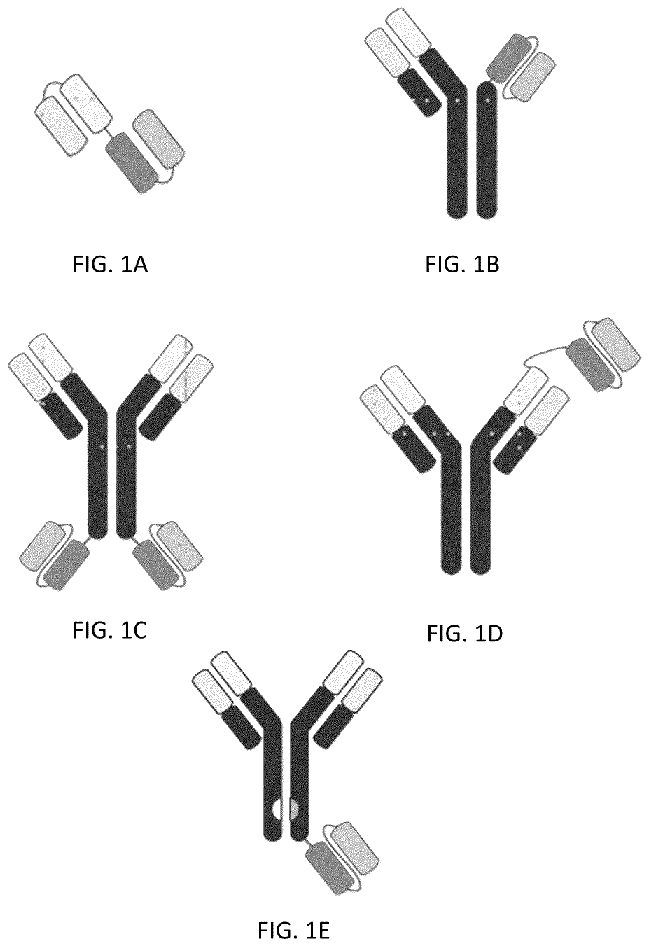

As provided for herein, the bispecific antibody comprising a CLDN6 binding domain and a CD3 binding domain is provided in various formats. Exemplary bispecific antibody formats are provided in FIG. 1.

In some embodiments, the bispecific antibody comprising a CLDN6 binding domain and a CD3 binding domain is a tandem scFv bispecific antibody, wherein the CLDN6 binding domain comprises a first V_{H} and a first V_{L} and the CD3 binding domain comprises a second V_{H} and a second V_{L}, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, (iii) the second V_{H} comprises an amino acid sequence having at least 90%, 91%, 92%, 93%, 94%, 95%, 96%, 97%, 98%, or 99% identity to the amino acid sequence of SEQ ID NO: 15 as provided for herein, and (iv) the second V_{L} comprises an amino acid sequence having at least 90%, 91%, 92%, 93%, 94%, 95%, 96%, 97%, 98%, or 99% identity to the amino acid sequence of SEQ ID NO: 16 as provided for herein.

In some embodiments, the bispecific antibody comprising a CLDN6 binding domain and a CD3 binding domain is a tandem scFv bispecific antibody, wherein the CLDN6 binding domain comprises a first V_{H} and a first V_{L} and the CD3 binding domain comprises a scFv, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, and (iii) the scFv comprises an amino acid sequence having at least 90%, 91%, 92%, 93%, 94%, 95%, 96%, 97%, 98%, or 99% identity to the amino acid sequence of SEQ ID NO: 32 as provided for herein.

In some embodiments, the bispecific antibody comprising a CLDN6 binding domain and a CD3 binding domain is an IgG-(scFv)₂ bispecific antibody, wherein the CLDN6 binding domain comprises a first V_{H} and a first V_{L} and the CD3 binding domain comprises a second V_{H} and a second V_{L}, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, (iii) the second V_{H} comprises an amino acid sequence having at least 90%, 91%, 92%, 93%, 94%, 95%, 96%, 97%, 98%, or 99% identity to the amino acid sequence of SEQ ID NO: 15 as provided for herein, and (iv) the second V_{L} comprises an amino acid sequence having at least 90%, 91%, 92%, 93%, 94%, 95%, 96%, 97%, 98%, or 99% identity to the amino acid sequence of SEQ ID NO: 16 as provided for herein.

In some embodiments, the bispecific antibody comprising a CLDN6 binding domain and a CD3 binding domain is a IgG-(scFv)₂ bispecific antibody, wherein the CLDN6 binding domain comprises a HC and a LC and the CD3 binding domain comprises a scFv, wherein (i) the HC comprises an amino acid sequence having at least 90%, 91%, 92%, 93%, 94%, 95%, 96%, 97%, 98%, or 99% identity to the amino acid sequence of SEQ ID NO: 17 as provided for herein, (ii) the LC comprises an amino acid sequence having at least 90%, 91%, 92%, 93%, 94%, 95%, 96%, 97%, 98%, or 99% identity to the amino acid sequence of SEQ ID NO: 18 as provided for herein, and (iii) the scFv comprises an amino acid sequence having at least 90%, 91%, 92%, 93%, 94%, 95%, 96%, 97%, 98%, or 99% identity to the amino acid sequence of SEQ ID NO: 32 as provided for herein.

In some embodiments, the bispecific antibody comprising a CLDN6 binding domain and a CD3 binding domain is an IgG-scFv bispecific antibody, wherein the CLDN6 binding domain comprises a first V_{H} and a first V_{L} and the CD3 binding domain comprises a second V_{H} and a second V_{L}, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, (iii) the second V_{H} comprises an amino acid sequence having at least 90%, 91%, 92%, 93%, 94%, 95%, 96%, 97%, 98%, or 99% identity to the amino acid sequence of SEQ ID NO: 15 as provided for herein, and (iv) the second V_{L} comprises an amino acid sequence having at least 90%, 91%, 92%, 93%, 94%, 95%, 96%, 97%, 98%, or 99% identity to the amino acid sequence of SEQ ID NO: 16 as provided for herein.

In some embodiments, the bispecific antibody comprising a CLDN6 binding domain and a CD3 binding domain is an IgG-scFv bispecific antibody, wherein the CLDN6 binding domain comprises a HC and a LC and the CD3 binding domain comprises a scFv, wherein (i) the HC comprises an amino acid sequence having at least 90%, 91%, 92%, 93%, 94%, 95%, 96%, 97%, 98%, or 99% identity to the amino acid sequence of SEQ ID NO: 17 as provided for herein, (ii) the LC comprises an amino acid sequence having at least 90%, 91%, 92%, 93%, 94%, 95%, 96%, 97%, 98%, or 99% identity to the amino acid sequence of SEQ ID NO: 18 as provided for herein, and (iii) the scFv comprises an amino acid sequence having at least 90%, 91%, 92%, 93%, 94%, 95%, 96%, 97%, 98%, or 99% identity to the amino acid sequence of SEQ ID NO: 32 as provided for herein. In some embodiments, the bispecific antibody comprising a CLDN6 binding domain and a CD3 binding domain is an IgG-scFv bispecific antibody, wherein the CLDN6 binding domain comprises a HC and a LC and the CD3 binding domain comprises a scFv, wherein the scFv is linked (e.g. through a polypeptide linker) to the constant domain of the HC of the CLDN6 binding domain. In some embodiments, the bispecific antibody comprising a CLDN6 binding domain and a CD3 binding domain is an IgG-scFv bispecific antibody, wherein the CLDN6 binding domain comprises a HC and a LC and the CD3 binding domain comprises a scFv, wherein the scFv is linked (e.g. through a polypeptide linker) to V_{H} domain of the HC of the CLDN6 binding domain.

In some embodiments, the bispecific antibody comprising a CLDN6 binding domain and a CD3 binding domain is an IgG-scFv bispecific antibody, wherein the CLDN6 binding domain comprises a HC and a LC and the CD3 binding domain comprises a scFv, wherein the scFv is linked (e.g. through a polypeptide linker) to the constant domain of the HC of the CLDN6 binding domain. In some embodiments, the bispecific antibody comprising a CLDN6 binding domain and a CD3 binding domain is an IgG-scFv bispecific antibody, wherein the CLDN6 binding domain comprises a HC and a LC and the CD3 binding domain comprises a scFv, wherein the scFv is linked (e.g. through a polypeptide linker) to the constant domain of the second HC of the CLDN6 binding domain. In some embodiments, the bispecific antibody comprising a CLDN6 binding domain and a CD3 binding domain is an IgG-scFv bispecific antibody, wherein the CLDN6 binding domain comprises a HC and a LC and the CD3 binding domain comprises a scFv, wherein the scFv is linked (e.g. through a polypeptide linker) to V_{H} domain of the first HC of the CLDN6 binding domain. In some embodiments, the bispecific antibody comprising a CLDN6 binding domain and a CD3 binding domain is an IgG-scFv bispecific antibody, wherein the CLDN6 binding domain comprises a HC and a LC and the CD3 binding domain comprises a scFv, wherein the scFv is linked (e.g. through a polypeptide linker) to V_{H} domain of the second HC of the CLDN6 binding domain.

In some embodiments, the bispecific antibody comprising a CLDN6 binding domain and a CD3 binding domain is a Fab-Fc-scFv bispecific antibody, wherein the CLDN6 binding domain comprises a first V_{H} and a first V_{L} and the CD3 binding domain comprises a second V_{H} and a second V_{L}, wherein (i) the first V_{H} comprises a HCDR1 comprising an amino acid sequence of SEQ ID NO: 1, a HCDR2 comprising an amino acid sequence of SEQ ID NO: 2, and a HCDR3 comprising an amino acid sequence of SEQ ID NO: 3 as provided for herein, (ii) the first V_{L} comprises a LCDR1 comprising an amino acid sequence of SEQ ID NO: 4, a LCDR2 comprising an amino acid sequence of SEQ ID NO: 5, and a LCDR3 comprising an amino acid sequence of SEQ ID NO: 6 as provided for herein, (iii) the second V_{H} comprises a HCDR1 comprising an amino acid sequence of SEQ ID NO: 9, a HCDR2 comprising an amino acid sequence of SEQ ID NO: 10, and a HCDR3 comprising an amino acid sequence of SEQ ID NO: 11 as provided for herein, and (iv) the second V_{L} comprises a LCDR1 comprising an amino acid sequence of SEQ ID NO: 12, a LCDR2 comprising an amino acid sequence of SEQ ID NO: 13, and a LCDR3 comprising an amino acid sequence of SEQ ID NO: 14 as provided for herein.

In some embodiments, the bispecific antibody comprising a CLDN6 binding domain and a CD3 binding domain is a Fab-Fc-scFv bispecific antibody, wherein the CLDN6 binding domain comprises a first V_{H} and a first V_{L} and the CD3 binding domain comprises a second V_{H} and a second V_{L}, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, (iii) the second V_{H} comprises an amino acid sequence having at least 90%, 91%, 92%, 93%, 94%, 95%, 96%, 97%, 98%, or 99% identity to the amino acid sequence of SEQ ID NO: 15 as provided for herein, and (iv) the second V_{L} comprises an amino acid sequence having at least 90%, 91%, 92%, 93%, 94%, 95%, 96%, 97%, 98%, or 99% identity to the amino acid sequence of SEQ ID NO: 16 as provided for herein.

In some embodiments, the bispecific antibody comprising a CLDN6 binding domain and a CD3 binding domain is a Fab-Fc-scFv bispecific antibody, wherein the CLDN6 binding domain comprises a first V_{H} and a first V_{L} and the CD3 binding domain comprises a second V_{H} and a second V_{L}, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7, provided that the first V_{H} comprises a HCDR1 comprising an amino acid sequence of SEQ ID NO: 1, a HCDR2 comprising an amino acid sequence of SEQ ID NO: 2, and a HCDR3 comprising an amino acid sequence of SEQ ID NO: 3 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8, provided that the first V_{L} comprises a LCDR1 comprising an amino acid sequence of SEQ ID NO: 4, a LCDR2 comprising an amino acid sequence of SEQ ID NO: 5, and a LCDR3 comprising an amino acid sequence of SEQ ID NO: 6 as provided for herein, (iii) the second V_{H} comprises an amino acid sequence having at least 90%, 91%, 92%, 93%, 94%, 95%, 96%, 97%, 98%, or 99% identity to the amino acid sequence of SEQ ID NO: 15, provided that the first V_{H} comprises a HCDR1 comprising an amino acid sequence of SEQ ID NO: 9, a HCDR2 comprising an amino acid sequence of SEQ ID NO: 10, and a HCDR3 comprising an amino acid sequence of SEQ ID NO: 11 as provided for herein, and (iv) the second V_{L} comprises an amino acid sequence having at least 90%, 91%, 92%, 93%, 94%, 95%, 96%, 97%, 98%, or 99% identity to the amino acid sequence of SEQ ID NO: 16, provided that the first V_{L} comprises a LCDR1 comprising an amino acid sequence of SEQ ID NO: 12, a LCDR2 comprising an amino acid sequence of SEQ ID NO: 13, and a LCDR3 comprising an amino acid sequence of SEQ ID NO: 14 as provided for herein.

In some embodiments, the bispecific antibody comprising a CLDN6 binding domain and a CD3 binding domain is a Fab-Fc-scFv bispecific antibody, wherein the CLDN6 binding domain comprises a first V_{H} and a first V_{L} and the CD3 binding domain comprises an scFv, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, and (iii) the scFv comprises an amino acid sequence having at least 90%, 91%, 92%, 93%, 94%, 95%, 96%, 97%, 98%, or 99% identity to the amino acid sequence of SEQ ID NO: 32 as provided for herein.

In some embodiments, the bispecific antibody comprising a CLDN6 binding domain and a CD3 binding domain is a Fab-Fc-scFv bispecific antibody, wherein the CLDN6 binding domain comprises a first V_{H} and a first V_{L} and the CD3 binding domain comprises an scFv and constant domain, wherein (i) the first V_{H} comprises an amino acid sequence having at least 90%, 91%, 92%, 93%, 94%, 95%, 96%, 97%, 98%, or 99% identity to the amino acid sequence of SEQ ID NO: 7 as provided for herein, (ii) the first V_{L} comprises an amino acid sequence having at least 90%, 91%, 92%, 93%, 94%, 95%, 96%, 97%, 98%, or 99% identity to the amino acid sequence of SEQ ID NO: 8 as provided for herein, and (iii) the scFv and constant domain comprises an amino acid sequence having at least 90%, 91%, 92%, 93%, 94%, 95%, 96%, 97%, 98%, or 99% identity to the amino acid sequence of SEQ ID NO: 19 as provided for herein.

In some embodiments, the bispecific antibody comprising a CLDN6 binding domain and a CD3 binding domain is a Fab-Fc-scFv bispecific antibody, wherein the CLDN6 binding domain comprises a HC and a first LC and the CD3 binding domain comprises an scFv, wherein (i) the HC comprises an amino acid sequence having at least 90%, 91%, 92%, 93%, 94%, 95%, 96%, 97%, 98%, or 99% identity to the amino acid sequence of SEQ ID NO: 17 as provided for herein, (ii) the LC comprises an amino acid sequence having at least 90%, 91%, 92%, 93%, 94%, 95%, 96%, 97%, 98%, or 99% identity to the amino acid sequence of SEQ ID NO: 18 as provided for herein, and (iii) the scFv comprises an amino acid sequence having at least 90%, 91%, 92%, 93%, 94%, 95%, 96%, 97%, 98%, or 99% identity to the amino acid sequence of SEQ ID NO: 32 as provided for herein.

In some embodiments, the bispecific antibody comprising a CLDN6 binding domain and a CD3 binding domain is a Fab-Fc-scFv bispecific antibody, wherein the CLDN6 binding domain comprises a HC and a LC and the CD3 binding domain comprises an scFv and constant domain, wherein (i) the HC comprises an amino acid sequence having at least 90%, 91%, 92%, 93%, 94%, 95%, 96%, 97%, 98%, or 99% identity to the amino acid sequence of SEQ ID NO: 17 as provided for herein, (ii) the LC comprises an amino acid sequence having at least 90%, 91%, 92%, 93%, 94%, 95%, 96%, 97%, 98%, or 99% identity to the amino acid sequence of SEQ ID NO: 18 as provided for herein, and (iii) the scFv and constant domain comprises an amino acid sequence having at least 90%, 91%, 92%, 93%, 94%, 95%, 96%, 97%, 98%, or 99% identity to the amino acid sequence of SEQ ID NO: 19 as provided for herein.

In some embodiments, the bispecific antibody comprising a CLDN6 binding domain and a CD3 binding domain is a Fab-Fc-scFv bispecific antibody comprising a first, a second, and a third polypeptide, wherein the first polypeptide comprises a first light chain comprising a first variable light chain region, wherein the first variable light chain region comprises a CDR1 sequence comprising the amino acid sequence of SEQ ID NO: 4, a CDR2 sequence comprising the amino acid sequence of SEQ ID NO: 5, and a CDR3 sequence comprising the amino acid sequence of SEQ ID NO: 6; the second polypeptide comprises a first heavy chain comprising a first variable heavy chain region and a constant domain, wherein the first variable heavy chain region comprises a CDR1 comprising the amino acid sequence of SEQ ID NO: 1, a CDR2 comprising the amino acid sequence of SEQ ID NO: 2, and a CDR3 comprising the amino acid sequence of SEQ ID NO: 2; and the third polypeptide comprises a second light chain and a second heavy chain and a constant domain, wherein the second heavy chain comprises a second variable heavy chain region comprising a CDR1 comprising the amino acid sequence of SEQ ID NO: 9, a CDR 2 comprising the amino acid sequence of SEQ ID NO: 10, and a CDR3 comprising the amino acid sequence of SEQ ID NO: 11, and the second light chain comprises a second variable light chain region comprising a CDR1 comprising the amino acid sequence of SEQ ID NO: 12, a CDR2 comprising the amino acid sequence of SEQ ID NO: 13, and a CDR3 comprising the amino acid sequence of SEQ ID NO: 14. In some embodiments, the CDRs of the first, second, and third polypeptides are variants of any of the foregoing as provided for herein. In some embodiments, the first polypeptide comprises an amino acid sequence that is at least 95% identical to the amino acid sequence of SEQ ID NO: 18. In some embodiments, the second polypeptide comprises an amino acid sequence that is at least 95% identical to the amino acid sequence of SEQ ID NO: 17. In some embodiments, the third polypeptide comprises an amino acid sequence that is at least 95% identical to the amino acid sequence of SEQ ID NO: 19.

In some embodiments, the bispecific antibody comprising a CLDN6 binding domain and a CD3 binding domain is a Fab-Fc-scFv bispecific antibody comprising a first, a second, and a third polypeptide, wherein the first polypeptide comprises an amino acid sequence of SEQ ID NO: 18, the second polypeptide comprises an amino acid sequence of SEQ ID NO: 17, and the third polypeptide comprises an amino acid sequence of SEQ ID NO: 19.

The foregoing embodiments describing the bispecific antibody comprising a CLDN6 binding domain and a CD3 binding domain are exemplary only and are not to be construed to be limiting in any way. Additional exemplary bispecific antibodies and bispecific antibody formats that are within the scope of the present disclosure may be found at least in U.S. Publication Nos. US 2022/0127355, US 2023/0220066A1, US 2023/0057904A1, and US 2024/0132584A1, U.S. Pat. Nos. 10,738,132B2, 10,968,276B2, 11,053,316B2, 11,248,046, and 11,739,144B2, and International Publication Serial Nos. WO 2022/192403A1, WO 2021/006328A1, WO 2021/200939A1, WO 2010/094499A1, WO 2020/168059A1, WO 2022/096700A1, WO 2023/05328A1, and WO 2023/054421A1, each of which are incorporated by reference herein in their entirety.

### Pharmaceutical Compositions

The compositions disclosed herein can comprise a pharmaceutical composition, and for example include a pharmaceutically acceptable carrier, and/or a pharmaceutical formulation.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the composition, preferably those with activities complementary to the composition, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the composition in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the composition, by multiple bolus administrations of the composition, or by continuous infusion administration of the composition. In some embodiments, the pharmaceutical composition does not include a chemotherapeutic.

Formulations include those for oral, intravenous, intraperitoneal, subcutaneous, pulmonary, transdermal, intramuscular, intranasal, buccal, sublingual, or suppository administration. In some embodiments, the composition is administered parenterally. The term “parenteral,” as used herein, includes intravenous, intramuscular, subcutaneous, rectal, vaginal, and intraperitoneal administration. In some embodiments, the composition is administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the composition in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

In some embodiments, a pharmaceutical composition is provided comprising a bispecific antibody as provided for herein. In some embodiments, the pharmaceutical composition comprises a bispecific antibody comprising a CLDN6 binding domain and a CD3 binding domain. In some embodiments, the pharmaceutical composition comprises a bispecific antibody comprising a CLDN6 binding domain as provided for herein and a CD3 binding domain as provided for herein. In some embodiments, the pharmaceutical composition comprising a bispecific antibody comprising a CLDN6 binding domain and a CD3 binding domain further comprises one or more pharmaceutically acceptable excipients.

In some embodiments, the pharmaceutical composition comprises a bispecific antibody comprising a CLDN6 binding domain and a CD3 binding domain, a pharmaceutically acceptable buffer, a sugar, and a non-ionic surfactant.

Non-limiting examples of buffers acceptable for use in the pharmaceutical composition include, but are not limited to, histidine buffer, HEPES, phosphoric acid buffer, citric acid buffer, acetic acid buffer, succinic acid buffer, phosphate buffer, acetate buffer, citrate buffer, succinate buffer, ascorbic acid buffer, glutamic acid buffer, lactic acid buffer, maleic acid buffer, trometamol buffer, or gluconic acid buffer, or any combination thereof. In some embodiments, the pharmaceutically acceptable buffer is histidine buffer. In some embodiments, the pharmaceutically acceptable buffer is HEPES. In some embodiments, the pharmaceutically acceptable buffer is phosphoric acid buffer. In some embodiments, the pharmaceutically acceptable buffer is citric acid buffer. In some embodiments, the pharmaceutically acceptable buffer is acetic acid buffer. In some embodiments, the pharmaceutically acceptable buffer is succinic acid buffer. In some embodiments, the pharmaceutically acceptable buffer is phosphate buffer. In some embodiments, the pharmaceutically acceptable buffer is acetate buffer. In some embodiments, the pharmaceutically acceptable buffer is citrate buffer. In some embodiments, the pharmaceutically acceptable buffer is succinate buffer. In some embodiments, the pharmaceutically acceptable buffer is ascorbic acid buffer. In some embodiments, the pharmaceutically acceptable buffer is glutamic acid buffer. In some embodiments, the pharmaceutically acceptable buffer is lactic acid buffer. In some embodiments, the pharmaceutically acceptable buffer is maleic acid buffer. In some embodiments, the pharmaceutically acceptable buffer is trometamol buffer. In some embodiments, the pharmaceutically acceptable buffer is gluconic acid buffer.

In some embodiments, the pharmaceutically acceptable buffer has a concentration of about 5 mM to about 100 mM. In some embodiments, the pharmaceutically acceptable buffer has a concentration of about 5 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, or any value or range in-between.

Non-limiting examples of sugars acceptable for use in the pharmaceutical composition include, but are not limited to, sucrose, trehalose, mannose, glucose, sorbitol, or mannitol, or any combination thereof. In some embodiments, the sugar is sucrose. In some embodiments, the sugar is trehalose. In some embodiments, the sugar is mannose. In some embodiments, the sugar is glucose. In some embodiments, the sugar is sorbitol. In some embodiments, the sugar is mannitol.

In some embodiments, the sugar has a concentration of about 50 mM to about 500 mM. In some embodiments, the sugar has a concentration of about 50 mM, about 100 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 350 mM, about 400 mM, about 450 mM, about 500 mM, or any value or range in-between.

Non-limiting examples of non-ionic surfactants acceptable for use in the pharmaceutical composition include, but are not limited to polyoxyethylensorbitan fatty acid esters (e.g., polysorbate 20 and polysorbate 80), polyethylene-polypropylene copolymers, polyethylene-polypropylene glycols, polyox ethylene-stearates, polyoxyethylene alkyl ethers, e.g., polyoxyethylene monolauryl ether, alkylphenylpolyoxyethylene ethers (Triton-X), polyoxyethylene-polyoxypropylene copolymer (Poloxamer, Pluronic), sodium dodecyl sulphate (SDS). In certain embodiments, the non-ionic surfactant is polysorbate 20, polysorbate 80, poloxamer, poloxamer 188, or poloxamer 407. In some embodiments, the non-ionic surfactant is polysorbate 20. In some embodiments, the non-ionic surfactant is polysorbate 80. In some embodiments, the non-ionic surfactant is a poloxamer. In some embodiments, the non-ionic surfactant is poloxamer 188. In some embodiments, the non-ionic surfactant is poloxamer 407.

In some embodiments, the non-ionic surfactant has a concentration of about 0.003% (w/v) to about 0.3% (w/v). In some embodiments, the non-ionic surfactant has a concentration of about 0.003% (w/v), about 0.004% (w/v), about 0.005% (w/v), about 0.006% (w/v), about 0.007% (w/v), about 0.008% (w/v), about 0.009% (w/v), about 0.01% (w/v), about 0.015% (w/v), about 0.02% (w/v), about 0.025% (w/v), about 0.03% (w/v), about 0.035% (w/v), about 0.04% (w/v), about 0.045% (w/v), about 0.05% (w/v), about 0.055% (w/v), about 0.06% (w/v), about 0.065% (w/v), about 0.07% (w/v), about 0.075% (w/v), about 0.08% (w/v), about 0.09% (w/v), about 0.1% (w/v), about 0.2% (w/v), about 0.3% (w/v), or any value or range in-between.

In some embodiments, the bispecific antibody is present at a concentration of about 1 mg/mL to about 50 mg/mL. In some embodiments, the bispecific antibody is present at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 25 mg/mL, about 30 mg/mL, about 35 mg/mL, about 40 mg/mL, about 45 mg/mL, about 50 mg/mL, or any value or range in-between.

In some embodiments, the pharmaceutical composition has a pH of about 5 to about 7. In some embodiments, the pharmaceutical composition has a pH of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, or any value or range in-between.

In some embodiments, the pharmaceutical composition comprises a bispecific antibody comprising a CLDN6 binding domain and a CD3 binding domain as provided for herein at a concentration of about 1 mg/mL to about 50 mg/mL, a pharmaceutically acceptable buffer at a concentration of about 5 mM to about 100 mM, a sugar at about 50 mM to about 500 mM, a non-ionic surfactant at a concentration of about 0.003% (w/v) to about 0.3% (w/v), and a pH of about 5 to about 7.

In some embodiments, the pharmaceutical composition comprises a bispecific antibody comprising a CLDN6 binding domain and a CD3 binding domain as provided for herein at a concentration of about 1 mg/mL to about 50 mg/mL, a histidine buffer at a concentration of about 5 mM to about 100 mM, sucrose at about 50 mM to about 500 mM, polysorbate 20 at a concentration of about 0.003% (w/v) to about 0.3% (w/v), and a pH of about 5 to about 7.

In some embodiments, the pharmaceutical composition comprises a bispecific antibody comprising a CLDN6 binding domain and a CD3 binding domain as provided for herein at a concentration of about 5 mg/mL to about 30 mg/mL, a histidine buffer at a concentration of about 5 mM to about 50 mM, sucrose at about 100 mM to about 350 mM, polysorbate 20 at a concentration of about 0.003% (w/v) to about 0.1% (w/v), and a pH of about 5.5 to about 6.5.

In some embodiments, the pharmaceutical composition comprises a bispecific antibody comprising a CLDN6 binding domain and a CD3 binding domain as provided for herein at a concentration of about 10 mg/mL to about 20 mg/mL, a histidine buffer at a concentration of about 10 mM to about 30 mM, sucrose at about 200 mM to about 300 mM, polysorbate 20 at a concentration of about 0.01% (w/v) to about 0.05% (w/v), and a pH of about 5.5 to about 6.5.

In one non-limiting embodiment, the pharmaceutical composition comprises a bispecific antibody comprising a CLDN6 binding domain and a CD3 binding domain as provided for herein at a concentration of about 15 mg/mL, a histidine buffer at a concentration of about 20 mM, sucrose at about 240 mM, polysorbate 20 at a concentration of about 0.03% (w/v), and a pH of about 6.0.

### Methods of Treatment

In some embodiments, methods of treating a cancer in a subject in need thereof are provided, the methods comprising administering to the subject a dose of a bispecific antibody as provided for herein.

In some embodiments, the method of treating a cancer in a subject with a bispecific antibody that binds to Claudin 6 (CLDN6) and CD3, the method comprising: administering to the subject a dose of a bispecific antibody wherein the dose is selected from the group consisting of about 10 μg to about 15000 μg, about 20 μg to about 13500 μg, about 22.5 μg to about 12800 μg, about 50 μg to about 10000 μg, about 70 μg to about 7500 μg, about 100 μg to about 6400 μg, about 200 μg to about 5000 μg, about 250 μg to about 4000 μg, about 300 μg to about 3200 μg, about 400 μg to about 2500 μg, about 500 μg to about 2000 μg, about 600 μg to about 1600 μg, or about 800 μg to about 1000 μg. In some embodiments, the method of treating a cancer in a subject with a bispecific antibody that binds to Claudin 6 (CLDN6) and CD3, the method comprising: administering to the subject a dose of a bispecific antibody wherein the dose is 22.5 μg. In some embodiments, the method of treating a cancer in a subject with a bispecific antibody that binds to Claudin 6 (CLDN6) and CD3, the method comprising: administering to the subject a dose of a bispecific antibody wherein the dose is 70 μg. In some embodiments, the method of treating a cancer in a subject with a bispecific antibody that binds to Claudin 6 (CLDN6) and CD3, the method comprising: administering to the subject a dose of a bispecific antibody wherein the dose is 140 μg. In some embodiments, the method of treating a cancer in a subject with a bispecific antibody that binds to Claudin 6 (CLDN6) and CD3, the method comprising: administering to the subject a dose of a bispecific antibody wherein the dose is 200 μg. In some embodiments, the method of treating a cancer in a subject with a bispecific antibody that binds to Claudin 6 (CLDN6) and CD3, the method comprising: administering to the subject a dose of a bispecific antibody wherein the dose is 250 μg. In some embodiments, the method of treating a cancer in a subject with a bispecific antibody that binds to Claudin 6 (CLDN6) and CD3, the method comprising: administering to the subject a dose of a bispecific antibody wherein the dose is 280 μg. In some embodiments, the method of treating a cancer in a subject with a bispecific antibody that binds to Claudin 6 (CLDN6) and CD3, the method comprising: administering to the subject a dose of a bispecific antibody wherein the dose is 400 μg. In some embodiments, the method of treating a cancer in a subject with a bispecific antibody that binds to Claudin 6 (CLDN6) and CD3, the method comprising: administering to the subject a dose of a bispecific antibody wherein the dose is 560 μg. In some embodiments, the method of treating a cancer in a subject with a bispecific antibody that binds to Claudin 6 (CLDN6) and CD3, the method comprising: administering to the subject a dose of a bispecific antibody wherein the dose is 800 μg. In some embodiments, the method of treating a cancer in a subject with a bispecific antibody that binds to Claudin 6 (CLDN6) and CD3, the method comprising: administering to the subject a dose of a bispecific antibody wherein the dose is 1000 μg. In some embodiments, the method of treating a cancer in a subject with a bispecific antibody that binds to Claudin 6 (CLDN6) and CD3, the method comprising: administering to the subject a dose of a bispecific antibody wherein the dose is 1120 μg. In some embodiments, the method of treating a cancer in a subject with a bispecific antibody that binds to Claudin 6 (CLDN6) and CD3, the method comprising: administering to the subject a dose of a bispecific antibody wherein the dose is 1600 μg. In some embodiments, the method of treating a cancer in a subject with a bispecific antibody that binds to Claudin 6 (CLDN6) and CD3, the method comprising: administering to the subject a dose of a bispecific antibody wherein the dose is 2240 μg. In some embodiments, the method of treating a cancer in a subject with a bispecific antibody that binds to Claudin 6 (CLDN6) and CD3, the method comprising: administering to the subject a dose of a bispecific antibody wherein the dose is 3200 μg. In some embodiments, the method of treating a cancer in a subject with a bispecific antibody that binds to Claudin 6 (CLDN6) and CD3, the method comprising: administering to the subject a dose of a bispecific antibody wherein the dose is 4480 μg. In some embodiments, the method of treating a cancer in a subject with a bispecific antibody that binds to Claudin 6 (CLDN6) and CD3, the method comprising: administering to the subject a dose of a bispecific antibody wherein the dose is 6400 μg. In some embodiments, the method of treating a cancer in a subject with a bispecific antibody that binds to Claudin 6 (CLDN6) and CD3, the method comprising: administering to the subject a dose of a bispecific antibody wherein the dose is 8960 μg. In some embodiments, the method of treating a cancer in a subject with a bispecific antibody that binds to Claudin 6 (CLDN6) and CD3, the method comprising: administering to the subject a dose of a bispecific antibody wherein the dose is 12800 μg.

In some embodiments, the method of treating a cancer in a subject with a bispecific antibody that binds to Claudin 6 (CLDN6) and CD3, the method comprising: administering a first dose of the bispecific antibody; and administering one or more subsequent doses of the bispecific antibody, wherein the first dose is less than or the same as the one or more subsequent doses. In some embodiments, the one or more subsequent doses of the bispecific antibody are at an amount that is the same as the first dose. In some embodiments, the one or more subsequent doses of the bispecific antibody are at an amount that is different than the first dose.

In some embodiments, the cancer is any cancer which displays increased CLDN6 expression. For the purposes of the present disclosure, a cancer which displays increased CLDN6 expression in at least 5% of the tumor cells by immunohistochemistry testing. In some embodiments, a cancer which displays increased CLDN6 expression expresses CLDN6 in at least 6% of the tumor cells by immunohistochemistry testing. In some embodiments, a cancer which displays increased CLDN6 expression expresses CLDN6 in at least 7% of the tumor cells by immunohistochemistry testing. In some embodiments, a cancer which displays increased CLDN6 expression expresses CLDN6 in at least 8% of the tumor cells by immunohistochemistry testing. In some embodiments, a cancer which displays increased CLDN6 expression expresses CLDN6 in at least 9% of the tumor cells by immunohistochemistry testing. In some embodiments, a cancer which displays increased CLDN6 expression expresses CLDN6 in at least 10% of the tumor cells by immunohistochemistry testing.

In some embodiments, the cancer to be treated by the methods herein is selected from ovarian cancer, testicular cancer, or endometrial cancer. In some embodiments, the cancer is ovarian cancer. In some embodiments, the cancer is ovarian cancer testicular cancer. In some embodiments, the cancer is ovarian cancer endometrial cancer. In some embodiments, the ovarian cancer is platinum-refractory/resistant ovarian cancer (PRROC).

In some embodiments, the method of treating a cancer with a bispecific antibody that binds to Claudin 6 (CLDN6) and CD3 comprises (i) administering to the subject a first dose of the bispecific antibody, wherein the first dose is selected from the group consisting of: about 10 μg to about 1500 μg, about 20 μg to about 1250 μg, about 22.5 μg to about 1000 μg, about 50 μg to about 800 μg, about 70 μg to about 750 μg, about 100 μg to about 500 μg, about 200 μg to about 400 μg, about 500 μg to about 1000 μg, about 600 μg to about 1000 μg, or about 800 μg to about 1000 μg, and (ii) administering to the subject one or more subsequent doses of the bispecific antibody, wherein each subsequent dose is selected from the group consisting of: about 10 μg to about 15000 μg, about 20 μg to about 13500 μg, about 22.5 μg to about 12800 μg, about 50 μg to about 10000 μg, about 70 μg to about 7500 μg, about 100 μg to about 6400 μg, about 200 μg to about 5000 μg, about 250 μg to about 4000 μg, about 300 μg to about 3200 μg, about 400 μg to about 2500 μg, about 500 μg to about 2000 μg, about 600 μg to about 1600 μg, or about 800 μg to about 1000 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody wherein the first dose is selected from the group consisting of: about 10 μg to about 1500 μg, about 20 μg to about 1250 μg, about 22.5 μg to about 1000 μg, about 50 μg to about 800 μg, about 70 μg to about 750 μg, about 100 μg to about 500 μg, or about 200 μg to about 400 μg, and (ii) administering to the subject one or more subsequent doses of the bispecific antibody, wherein each subsequent dose is selected from the group consisting of: about 10 μg to about 1500 μg, about 10 μg to about 1250 μg, about 10 μg to about 1000 μg, about 10 μg to about 750 μg, about 10 μg to about 500 μg, about 10 μg to about 400 μg, about 10 μg to about 200 μg, about 10 μg to about 100 μg, about 10 μg to about 70 μg, about 10 μg to about 50 μg, about 10 μg to about 22.5 μg, or about 10 μg to about 20 μg, about 10 μg to about 1500 μg, about 20 μg to about 1500 μg, about 22.5 μg to about 1500 μg, about 50 μg to about 1500 μg, about 70 μg to about 1500 μg, about 100 μg to about 1500 μg, about 200 μg to about 1500 μg, about 400 μg to about 1500 μg, about 500 μg to about 1500 μg, about 750 μg to about 1500 μg, about 1000 μg to about 1500 μg, or about 1250 μg to about 1500 μg, about 10 μg to about 15000 μg, about 20 μg to about 13500 μg, about 22.5 μg to about 12800 μg, about 50 μg to about 10000 μg, about 70 μg to about 7500 μg, about 100 μg to about 6400 μg, about 200 μg to about 5000 μg, about 250 μg to about 4000 μg, about 300 μg to about 3200 μg, about 400 μg to about 2500 μg, about 500 μg to about 2000 μg, about 600 μg to about 1600 μg, about 800 μg to about 1000 μg, about 250 μg to about 10000 μg, about 550 μg to about 10000 μg, about 1100 μg to about 10000 μg, about 2000 μg to about 10000 μg, or about 4000 μg to about 10000 μg or any value or range in-between. In certain embodiments, the bispecific antibody is administered intravenously, subcutaneously, or intramuscularly.

In some embodiments, the first dose of the bispecific antibody is at an amount of about 10 μg, about 20 μg, about 22.5 μg, about 50 μg, about 70 μg, about 100 μg, about 140 μg, about 200 μg, about 280 μg, about 400 μg, about 500 μg, about 560 μg, about 750 μg, about 800 μg, about 1000 μg, about 1250 μg, or about 1500 μg, or any value or range in-between.

In some embodiments, the first dose of the bispecific antibody is at an amount of about 22.5 μg. In some embodiments, the first dose of the bispecific antibody is at an amount of about 70 μg. In some embodiments, the first dose of the bispecific antibody is at an amount of about 140 μg. In some embodiments, the first dose of the bispecific antibody is at an amount of about 200 μg. In some embodiments, the first dose of the bispecific antibody is at an amount of about 280 μg. In some embodiments, the first dose of the bispecific antibody is at an amount of about 400 μg. In some embodiments, the first dose of the bispecific antibody is at an amount of about 560 μg. In some embodiments, the first dose of the bispecific antibody is at an amount of about 800 μg. In some embodiments, the first dose of the bispecific antibody is at an amount of about 1000 μg.

In some embodiments, the one or more subsequent doses of the bispecific antibody are at an amount of about 10 μg to about 15000 μg, about 10 μg to about 13500 μg, about 10 μg to about 12800 μg, about 10 μg to about 10000 μg, about 10 μg to about 7500 μg, about 10 μg to about 6400 μg, about 10 μg to about 5000 μg, about 10 μg to about 4000 μg, about 10 μg to about 3200 μg, about 10 μg to about 2500 μg, about 10 μg to about 2000 μg, about 10 μg to about 1600 μg, about 10 μg to about 1000 μg, about 10 μg to about 800 μg, about 10 μg to about 600 μg, about 10 μg to about 500 μg, about 10 μg to about 400 μg, about 10 μg to about 300 μg, about 10 μg to about 250 μg, about 10 μg to about 200 μg, about 10 μg to about 100 μg, about 10 μg to about 70 μg, about 10 μg to about 50 μg, about 10 μg to about 22.5 μg, or about 10 μg to about 20 μg, about 10 μg to about 15000 μg, about 20 μg to about 15000 μg, about 22.5 μg to about 15000 μg, about 50 μg to about 15000 μg, about 70 μg to about 15000 μg, about 100 μg to about 15000 μg, about 200 μg to about 15000 μg, about 250 μg to about 15000 μg, about 300 μg to about 15000 μg, about 400 μg to about 15000 μg, about 500 μg to about 15000 μg, about 600 μg to about 15000 μg, about 800 μg to about 15000 μg, about 1000 μg to about 15000 μg, about 1600 μg to about 15000 μg, about 2000 μg to about 15000 μg, about 2500 μg to about 15000 μg, about 3200 μg to about 15000 μg, about 4000 μg to about 15000 μg, about 5000 μg to about 15000 μg, about 6400 μg to about 15000 μg, about 7500 μg to about 15000 μg, about 10000 μg to about 15000 μg, about 12800 μg to about 15000 μg, or about 13500 μg to about 15000 μg, or any value or range in-between.

In some embodiments, the one or more subsequent doses of the bispecific antibody are at an amount of about 10 μg, about 20 μg, about 22.5 μg, about 50 μg, about 70 μg, about 100 μg, about 140 μg, about 200 μg, about 250 μg, about 280 μg, about 300 μg, about 400 μg, about 500 μg, about 560 μg, about 600 μg, about 800 μg, about 1000 μg, about 1120 μg, about 1600 μg, about 2000 μg, about 22400 μg, about 2500 μg, about 3200 μg, about 4000 μg, about 4480 μg, about 5000 μg, about 6400 μg, about 7500 μg, about 8960 μg, about 10000 μg, about 12800 μg, about 13500 μg, or about 15000 μg, or any value or range in-between.

In some embodiments, the one or more subsequent doses of the bispecific antibody are at an amount of about 22.5 μg. In some embodiments, the one or more subsequent doses of the bispecific antibody are at an amount of about 70 μg. In some embodiments, the one or more subsequent doses of the bispecific antibody are at an amount of about 140 μg. In some embodiments, the one or more subsequent doses of the bispecific antibody are at an amount of about 200 μg. In some embodiments, the one or more subsequent doses of the bispecific antibody are at an amount of about 280 μg. In some embodiments, the one or more subsequent doses of the bispecific antibody are at an amount of about 400 μg. In some embodiments, the one or more subsequent doses of the bispecific antibody are at an amount of about 560 μg. In some embodiments, the one or more subsequent doses of the bispecific antibody are at an amount of about 800 μg. In some embodiments, the one or more subsequent doses of the bispecific antibody are at an amount of about 1120 μg. In some embodiments, the one or more subsequent doses of the bispecific antibody are at an amount of about 1600 μg. In some embodiments, the one or more subsequent doses of the bispecific antibody are at an amount of about 2240 μg. In some embodiments, the one or more subsequent doses of the bispecific antibody are at an amount of about 3200 μg. In some embodiments, the one or more subsequent doses of the bispecific antibody are at an amount of about 4480 μg. In some embodiments, the one or more subsequent doses of the bispecific antibody are at an amount of about 6400 μg. In some embodiments, the one or more subsequent doses of the bispecific antibody are at an amount of about 8960 μg. In some embodiments, the one or more subsequent doses of the bispecific antibody are at an amount of about 12800 μg.

In some embodiments, the method of treating a cancer with a bispecific antibody that binds to Claudin 6 (CLDN6) and CD3 comprises (i) administering to the subject a first dose of the bispecific antibody, wherein the first dose is about 22.5 μg, and (ii) administering to the subject one or more subsequent doses of the bispecific antibody, wherein each subsequent dose is about 22.5 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 22.5 μg, and (ii) administering to the subject one or more subsequent doses of the bispecific antibody, wherein each subsequent dose is about 70 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 70 μg, and (ii) administering to the subject one or more subsequent doses of the bispecific antibody, wherein each subsequent dose is about 140 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 70 μg, and (ii) administering to the subject one or more subsequent doses of the bispecific antibody, wherein each subsequent dose is about 200 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 140 μg, and (ii) administering to the subject one or more subsequent doses of the bispecific antibody, wherein each subsequent dose is about 280 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 200 μg, and (ii) administering to the subject one or more subsequent doses of the bispecific antibody, wherein each subsequent dose is about 400 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 280 μg, and (ii) administering to the subject one or more subsequent doses of the bispecific antibody, wherein each subsequent dose is about 560 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 400 μg, and (ii) administering to the subject one or more subsequent doses of the bispecific antibody, wherein each subsequent dose is about 800 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 560 μg, and (ii) administering to the subject one or more subsequent doses of the bispecific antibody, wherein each subsequent dose is about 1120 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 800 μg, and (ii) administering to the subject one or more subsequent doses of the bispecific antibody, wherein each subsequent dose is about 1600 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 1000 μg, and (ii) administering to the subject one or more subsequent doses of the bispecific antibody, wherein each subsequent dose is about 2240 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 1000 μg, and (ii) administering to the subject one or more subsequent doses of the bispecific antibody, wherein each subsequent dose is about 3200 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 1000 μg, and (ii) administering to the subject one or more subsequent doses of the bispecific antibody, wherein each subsequent dose is about 4480 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 1000 μg, and (ii) administering to the subject one or more subsequent doses of the bispecific antibody, wherein each subsequent dose is about 6400 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 1000 μg, and (ii) administering to the subject one or more subsequent doses of the bispecific antibody, wherein each subsequent dose is about 8960 μg, wherein the bispecific antibody is as provided for herein. In certain embodiments, the bispecific antibody is administered intravenously, subcutaneously, or intramuscularly.

In some embodiments, the method of treating a cancer with a bispecific antibody that binds to Claudin 6 (CLDN6) and CD3 comprises (i) administering to the subject a first dose of the bispecific antibody, wherein the first dose is about 1000 μg, and (ii) administering to the subject one or more subsequent doses of the bispecific antibody, wherein each subsequent dose is about 12800 μg, wherein the bispecific antibody is as provided for herein. In certain embodiments, the bispecific antibody is administered intravenously, subcutaneously, or intramuscularly.

In some embodiments, at least one subsequent dose of the one or more subsequent doses is administered 7 days, 8 days, 9 days, 10 days, 11 days, 12 days, 13 days, or 14 days after the first dose. In some embodiments, at least one subsequent dose of the one or more subsequent doses is administered one, two, three, four, five, six, seven, eight, nine, ten, eleven, twelve, or more than twelve weeks after the first dose.

In some embodiments, at least one, two, three, four, five, six, seven, eight, nine, ten, eleven, twelve, or more than twelve subsequent doses are administered to the subject.

In some embodiments, the method comprises (i) administering to the subject a first dose of a bispecific antibody and (ii) administering to the subject one or more subsequent doses of the bispecific antibody, wherein the first of the one or more subsequent doses (i.e. the second dose) is administered one week after the first dose. In some embodiments, the method comprises (i) administering to the subject a first dose of a bispecific antibody and (ii) administering to the subject one or more subsequent doses of the bispecific antibody, wherein the first of the one or more subsequent doses (i.e. the second dose) is administered two weeks after the first dose. In some embodiments, the method comprises (i) administering to the subject a first dose of a bispecific antibody and (ii) administering to the subject one or more subsequent doses of the bispecific antibody, wherein starting with the second of the one or more subsequent doses (i.e. the third dose) each subsequent dose is administered one week after a previous dose. In certain embodiments, the bispecific antibody is administered intravenously, subcutaneously, or intramuscularly.

In some embodiments, the method comprises (i) intravenously administering to the subject a first dose of a bispecific antibody and (ii) intravenously administering to the subject one or more subsequent doses of the bispecific antibody, wherein the bispecific antibody is administered over a period of about 10 minutes to about 30 minutes, about 10 minutes to about 20 minutes, or about 12 minutes to about 18 minutes. In some embodiments, the method comprises (i) intravenously administering to the subject a first dose of a bispecific antibody and (ii) intravenously administering to the subject one or more subsequent doses of the bispecific antibody, wherein the bispecific antibody is administered over a period of about 10, 11, 12, 13, 14, 15, 16, 17, 18, 19, 20, 21, 22, 23, 24, 25, 26, 27, 28, 29, or 30 minutes.

In some embodiments, the method of treating a cancer with a bispecific antibody that binds to Claudin 6 (CLDN6) and CD3 comprises (i) administering to the subject the bispecific antibody as provided for herein and (ii) administering to the subject a corticosteroid, an antihistamine, or a combination thereof. In some embodiments, the corticosteroid, antihistamine, or combination thereof are administered prior to the first dose of the bispecific antibody. In some embodiments, the corticosteroid, antihistamine, or combination thereof is administered prior to the administration of the bispecific antibody as provided for herein. In some embodiments, the corticosteroid, antihistamine, or combination thereof are administered at the same time as the bispecific antibody as provided for herein. In some embodiments, the corticosteroid, antihistamine, or combination thereof are each, independently, administered orally or intravenously.

In some embodiments, the corticosteroid is administered at a concentration of about 1 mg to about 50 mg, about 5 mg to about 40 mg, about 5 mg to about 30 mg, about 10 mg to about 30 mg, about 10 mg to about 25 mg, about 10 mg to about 20 mg, or about 12 mg to about 20 mg. In some embodiments, the corticosteroid is administered at a concentration of about 1, 2, 4, 5, 10, 11, 12, 13, 14, 15, 16, 17, 18, 19, 20, 25, 30, 35, 40, 45, or 50 mg. In some embodiments, the corticosteroid is administered at a concentration of about 10 mg. In some embodiments, the corticosteroid is administered at a concentration of about 12 mg. In some embodiments, the corticosteroid is administered at a concentration of about 14 mg. In some embodiments, the corticosteroid is administered at a concentration of about 16 mg. In some embodiments, the corticosteroid is administered at a concentration of about 18 mg. In some embodiments, the corticosteroid is administered at a concentration of about 20 mg.

In some embodiments, the antihistamine is administered at a concentration of about 1 mg to about 100 mg, about 10 mg to about 90 mg, about 20 mg to about 80 mg, about 25 mg to about 75 mg, about 30 mg to about 70 mg, about 35 mg to about 65 mg, about 40 mg to about 60 mg, or about 45 mg to about 55 mg. In some embodiments, the antihistamine is administered at a concentration of about 10, 20, 30, 40, 45, 50, 55, 60, 65, 70, 80, 90, or 100 mg. In some embodiments, the antihistamine is administered at a concentration of about 40 mg. In some embodiments, the antihistamine is administered at a concentration of about 45 mg. In some embodiments, the antihistamine is administered at a concentration of about 50 mg. In some embodiments, the antihistamine is administered at a concentration of about 55 mg. In some embodiments, the antihistamine is administered at a concentration of about 60 mg.

In some embodiments, the method of treating a cancer in a subject in need thereof comprises (i) intravenously administering to the subject a first dose of a bispecific antibody wherein the first dose is selected from the group consisting of: about 22.5 μg, about 70 μg, about 140 μg, about 200 μg, about 280 μg, about 400 μg, about 560 μg, about 800 μg, or about 1000 μg, and (ii) intravenously administering to the subject one or more subsequent doses, wherein each subsequent dose is selected from the group consisting of: about 22.5 μg, about 70 μg, about 140 μg, about 200 μg, about 280 μg, about 400 μg, about 560 μg, about 800 μg, about 1120 μg, about 1600 μg, about 2240 μg, about 3200 μg, about 4480 μg, about 6400 μg, about 8960 μg, or about 12800 μg, wherein the bispecific antibody is as provided for herein.

In some embodiments, treatment of a subject via the methods provided for herein results in a Cₘₐₓ of the bispecific antibody approximately equivalent to 0.04 nM.

In some embodiments, treatment of a subject via the methods provided for herein results in a Cₘₐₓ of the bispecific antibody of at least 0.04 nM

In some embodiments, treatment of a subject via the methods provided for herein results in an area under the curve (AUC) for the bispecific antibody of approximately 175 hr*ng/mL.

In some embodiments, treatment of a subject via the methods provided for herein results in an area under the curve (AUC) for the bispecific antibody of at least 175 hr*ng/mL.

In some embodiments, treatment of a subject via the methods provided for herein results in minimal accumulation of anti-drug antibodies.

The activity of a bispecific antibody as provided for herein may be assessed by any methodology known. In certain embodiment, the activity of the bispecific antibody is assessed via RECIST (Response Evaluation Criteria in Solid Tumors) Guidelines. The RECIST Guidelines are a published set of rules that define when tumors in cancer patients improve (“respond”), stay the same (“stabilize”), or worsen (“progress”) during treatment. For the context of the present disclosure, the RECIST Guidelines are as shown in the table below:

| Target | Non-target | New | |
|---|---|---|---|
| Lesions | Lesions | Lesions | Overall Response |
| CR | CR | No | Complete Response |
| CR | Non-CR/non-PD | No | Partial Response |
| PR | Non-CR/non-PD | No | Partial Response |
| SD | Non-CR/non-PD | No | Stable Disease |
| PD | Any | Yes/No | Progressive Disease |
| Any | PD | Yes/No | Progressive Disease |
| Any | Any | Yes | Progressive Disease |

CR = complete response
PR = partial response
SD = Stable Disease
PD = Progressive Disease

In some embodiments, treatment of a subject via the methods provided for herein results in a complete response per RECIST Guidelines.

In some embodiments, treatment of a subject via the methods provided for herein results in a partial response per RECIST Guidelines.

In some embodiments, treatment of a subject via the methods provided for herein results in a stable disease response per RECIST Guidelines.

In certain embodiment, the activity of the bispecific antibody is assessed via Immune RECIST Guidelines. The RECIST Guidelines are a published set of rules that define when tumors in cancer patients improve (“respond”), stay the same (“stabilize”), or worsen (“progress”) during treatment with immunotherapeutics. For the context of the present disclosure, the Immune RECIST Guidelines are as shown in the table below:

Time-Point Response

| | | | Time Point Response | |
|---|---|---|---|---|
| Target | Non-Target | New | No prior | Prior |
| Lesions* | Lesions* | Lesions* | iUPD** | iUPD**, *** |
| iCR | iCR | No | iCR | iCR |
| iCR | Non-iCR/Non- | No | iPR | iPR |
| | iUPD | | | |
| iPR | Non-iCR/Non- | No | iPR | iPR |
| | iUPD | | | |
| iSD | Non-iCR/Non- | No | iSD | iSD |
| | iUPD | | | |
| iUPD with | iUPD with no | Yes | NA | NLs confirms iCPD if NLs were |
| no change | change OR | | | previously identified and increase in |
| | | | | size (≥5 mm in SOM for NLT or any |
| | | | | increase for NLNT) or number; If no |
| OR decrease | decrease from | | | change in NLs (size or number) from |
| from last TP | last TP | | | last TP, remains iUPD |
| iSD, iPR, | iUPD | No | iUPD | Remains iUPD unless iCPD confirmed |
| iCR | | | | based in further increase in size of NT |
| | | | | disease (need not meet RECIST 1.1 |
| | | | | criteria for unequivocal PD) |
| iUPD | Non-iCR/ Non- | No | iUPD | Remains iUPD unless iCPD confirmed |
| | iUPD; iCR | | | based on: further increase in SOM of |
| | | | | at least 5 mm, otherwise remains iUPD |
| iUPD | iUPD | No | iUPD | Remains iUPD unless iCPD confirmed |
| | | | | based on further increase in: |
| | | | | previously identified T lesion iUPD in |
| | | | | SOM ≥5 mm and/or |
| | | | | NT lesion iUPD (prior assessment - |
| | | | | need not be unequivocal PD) |
| iUPD | iUPD | Yes | iUPD | Remains iUPD unless iCPD confirmed |
| | | | | based on further increase in: |
| | | | | previously identified T lesion iUPD |
| | | | | in SOM ≥5 mm and/or |
| | | | | previously identified NT lesion iUPD |
| | | | | (need not be unequivocal) and/or |
| | | | | size or number of new lesions |
| | | | | previously identified |
| Non- | Non-iUPD/PD | Yes | iUPD | Remains iUPD unless iCPD confirmed |
| iUPD/PD | | | | based on: |
| | | | | Increase in size or number of new |
| | | | | lesions previously identified |

*Using RECIST 1.1 principles. If no PSPD occurs, RECIST 1.1 and iRECIST categories for CR, PR, and DS would be the same.
**in any lesion category.
*** previously identified in assessment immediately prior to this TP.
iCR = immune complete response; iPR = immune partial response; iSD—immune stable disease; iUPD—immune unconfirmed progression; iCPD—immune confirmed progression; NL—new lesion; NLT—new target lesion; NLNT—new lesion non target; TP—time point.

In some embodiments, treatment of a subject via the methods provided for herein results in a complete response per Immune RECIST Guidelines.

In some embodiments, treatment of a subject via the methods provided for herein results in a partial response per Immune RECIST Guidelines.

In some embodiments, treatment of a subject via the methods provided for herein results in a stable disease response per Immune RECIST Guidelines.

In some embodiments, treatment of a subject via the methods provided for herein results in an increase in levels of one or more cytokines in the subject selected from IL-2, IL-6, IL-8, IL-10, MCP1, IFNγ, TNFα, GM-CSF and CRP.

In some embodiments, treatment of a subject via the methods provided for herein results in an increase in levels of one or more of CD3+ T cells, CD4+ T cells, CD*+ T cells, CD69+ T cells, or Ki67+ T cells in the subject.

In some embodiments, treatment of a subject via the methods provided for herein results in an increase in levels of one or more of T cells, B cells, natural killer (NK) cells, monocytes, macrophages, neutrophils, eosinophils, basophils, mast cells, or dendritic cells in the subject.

In some embodiments, treatment of a subject via the methods provided for herein results in a decrease in CLDN6 expression in the tumor microenvironment.

In some embodiments, treatment of a subject via the methods provided for herein results in a change in the number and kind of infiltrating lymphocytes (TILs).

In some embodiments, treatment of a subject via the methods provided for herein results in a decrease in tumor size. In some embodiments, treatment of a subject via the methods provided for herein results in complete destruction or depletion of the tumor.

In some embodiments, treatment of a subject via the methods provided for herein results in depletion of tumor cells in the subject. In some embodiments, treatment of a subject via the methods provided for herein results in complete depletion of tumor cells in the subject.

In some embodiments, treatment of a subject via the methods provided for herein results in an increased survival rate of the subject over a period of time as compared to subjects that do not receive the bispecific antibody. In some embodiments, treatment of a subject via the methods provided for herein results the subject showing no signs of cancer.

### ENUMERATED EMBODIMENTS

In some embodiments, the following embodiments are provided:

1. A method of treating a cancer in a subject with a bispecific antibody that binds to Claudin 6 (CLDN6) and CD3, the method comprising:
administering a first dose of the bispecific antibody; and
administering one or more subsequent doses of the bispecific antibody,
wherein the first dose is less than or the same as the one or more subsequent doses.

2. The method of embodiment 1,
wherein the first dose of the bispecific antibody is selected from the group consisting of: about 10 μg to about 1500 μg, about 20 μg to about 1250 μg, about 22.5 μg to about 1000 μg, about 50 μg to about 800 μg, about 70 μg to about 750 μg, about 100 μg to about 500 μg, about 200 μg to about 400 μg, about 500 μg to about 1000 μg, about 600 μg to about 1000 μg, or about 800 μg to about 1000 μg, and
wherein the one or more subsequent dose of the bispecific antibody is selected from the group consisting of: about 10 μg to about 15000 μg, about 20 μg to about 13500 μg, about 22.5 μg to about 12800 μg, about 50 μg to about 10000 μg, about 70 μg to about 7500 μg, about 100 μg to about 6400 μg, about 200 μg to about 5000 μg, about 250 μg to about 4000 μg, about 300 μg to about 3200 μg, about 400 μg to about 2500 μg, about 500 μg to about 2000 μg, about 600 μg to about 1600 μg, about 800 μg to about 1000 μg, about 250 μg to about 10000 μg, about 550 μg to about 10000 μg, about 1100 μg to about 10000 μg, about 2000 μg to about 10000 μg, or about 4000 μg to about 10000 μg.

3. The method of embodiment 1 or embodiment 2, wherein the first dose is about 22.5 μg.

4. The method of embodiment 1 or embodiment 2, wherein the first dose is about 70 μg.

5. The method of embodiment 1 or embodiment 2, wherein the first dose is about 140 μg.

6. The method of embodiment 1 or embodiment 2, wherein the first dose is about 200 μg.

7. The method of embodiment 1 or embodiment 2, wherein the first dose is about 280 μg.

8. The method of embodiment 1 or embodiment 2, wherein the first dose is about 400 μg.

9. The method of embodiment 1 or embodiment 2, wherein the first dose is about 560 μg.

10. The method of embodiment 1 or embodiment 2, wherein the first dose is about 800 μg.

11. The method of embodiment 1 or embodiment 2, wherein the first dose is about 1000 μg.

12. The method of any one of embodiments 1-11, wherein the one or more subsequent doses are the same amount as the first dose.

13. The method of any one of embodiments 1-11, wherein the one or more subsequence doses are in an amount different from the first dose.

14. The method of any one of embodiments 1-13, wherein the one or more subsequent doses is about 22.5 μg.

15. The method of any one of embodiments 1-13, wherein the one or more subsequent doses is about 70 μg.

16. The method of any one of embodiments 1-13, wherein the one or more subsequent doses is about 140 μg.

17. The method of any one of embodiments 1-13, wherein the one or more subsequent doses is about 200 μg.

18. The method of any one of embodiments 1-13, wherein the one or more subsequent doses is about 280 μg.

19. The method of any one of embodiments 1-13, wherein the one or more subsequent doses is about 400 μg.

20. The method of any one of embodiments 1-13, wherein the one or more subsequent doses is about 560 μg.

21. The method of any one of embodiments 1-13, wherein the one or more subsequent doses is about 800 μg.

22. The method of any one of embodiments 1-13, wherein the one or more subsequent doses is about 1120 μg.

23. The method of any one of embodiments 1-13, wherein the one or more subsequent doses is about 1600 μg.

24. The method of any one of embodiments 1-13, wherein the one or more subsequent doses is about 2240 μg.

25. The method of any one of embodiments 1-13, wherein the one or more subsequent doses is about 3200 μg.

26. The method of any one of embodiments 1-13, wherein the one or more subsequent doses is about 4480 μg.

27. The method of any one of embodiments 1-13, wherein the one or more subsequent doses is about 6400 μg.

28. The method of any one of embodiments 1-13, wherein the one or more subsequent doses is about 8960 μg.

29. The method of any one of embodiments 1-13, wherein the one or more subsequent doses is about 12800 μg.

30. The method of any one of embodiments 1-29, wherein the one or more subsequent doses is administered one, two, three, four, five, six, seven, eight, nine, ten, eleven, twelve, or more than twelve weeks after the first dose.

31. The method of any one of embodiments 1-30, wherein at least one, two, three, four, five, six, seven, eight, nine, ten, eleven, twelve, or more than twelve subsequent doses are administered to the subject.

32. The method of any one of embodiments 1-31, wherein each subsequent dose is administered one week after a previous dose.

33. The method of any one of embodiments 1-32, wherein the one or more subsequent dose is administered one week after the first dose.

34. The method of any one of embodiments 1-32, wherein the one or more subsequent dose is administered two weeks after the first dose.

35. The method of any one of embodiments 1-34, wherein the bispecific antibody is intravenously administered over a period of about 10 minutes to about 30 minutes, about 10 minutes to about 20 minutes, or about 12 minutes to about 18 minutes.

36. The method of any one of embodiments 1-35, wherein the bispecific antibody is administered as part of a pharmaceutically acceptable composition comprising the bispecific antibody, a pharmaceutically acceptable buffer, a sugar, and a non-ionic surfactant.

37. The method of any one of embodiments 1-36, wherein the bispecific antibody comprises a CLDN6 binding domain and a CD3 binding domain,
wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and
wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

38. The method of any one of embodiments 1-37, wherein the CLDN6 binding domain comprises a first VH comprising an amino acid sequence having at least 90% identity to the amino acid sequence of SEQ ID NO: 7, and comprises a first VL comprising an amino acid sequence having at least 90% identity to the amino acid sequence of SEQ ID NO: 8.

39. The method of any one of embodiments 1-38, wherein the CLDN6 binding domain comprises a first VH comprising an amino acid sequence having at least 95% identity to the amino acid sequence of SEQ ID NO: 7, and comprises a first VL comprising an amino acid sequence having at least 95% identity to the amino acid sequence of SEQ ID NO: 8.

40. The method of any one of embodiments 1-39, wherein the CLDN6 binding domain comprises a first VH comprising an amino acid sequence having at least 98% identity to the amino acid sequence of SEQ ID NO: 7, and comprises a first VL comprising an amino acid sequence having at least 98% identity to the amino acid sequence of SEQ ID NO: 8.

41. The method of any one of embodiments 1-40, wherein the CLDN6 binding domain comprises a first VH comprising the amino acid sequence of SEQ ID NO: 7, and comprises a first VL comprising the amino acid sequence of SEQ ID NO: 8.

42. The method of any one of embodiments 1-41, wherein the CLDN6 binding domain comprises a heavy chain (HC) and a light chain (LC), wherein the HC comprises an amino acid sequence having at least 90% identity to the amino acid sequence of SEQ ID NO: 17 and the LC comprises an amino acid sequence having at least 90% identity to the amino acid sequence of SEQ ID NO: 18.

43. The method of any one of embodiments 1-42, wherein the CLDN6 binding domain comprises a HC and a LC, wherein the HC comprises an amino acid sequence having at least 95% identity to the amino acid sequence of SEQ ID NO: 17 and the LC comprises an amino acid sequence having at least 95% identity to the amino acid sequence of SEQ ID NO: 18.

44. The method of any one of embodiments 1-43, wherein the CLDN6 binding domain comprises a HC and a LC, wherein the HC comprises an amino acid sequence having at least 98% identity to the amino acid sequence of SEQ ID NO: 17 and the LC comprises an amino acid sequence having at least 98% identity to the amino acid sequence of SEQ ID NO: 18.

45. The method of any one of embodiments 1-44, wherein the CLDN6 binding domain comprises a HC and a LC, wherein the HC comprises the amino acid sequence of SEQ ID NO: 17 and the LC comprises the amino acid sequence of SEQ ID NO: 18.

46. The method of any one of embodiments 1-45, wherein the CD3 binding domain comprises a second VH comprising an amino acid sequence having at least 90% identity to the amino acid sequence of SEQ ID NO: 15, and comprises a second VL comprising an amino acid sequence having at least 90% identity to the amino acid sequence of SEQ ID NO: 16.

47. The method of any one of embodiments 1-46, wherein the CD3 binding domain comprises a second VH comprising an amino acid sequence having at least 95% identity to the amino acid sequence of SEQ ID NO: 15, and comprises a second VL comprising an amino acid sequence having at least 95% identity to the amino acid sequence of SEQ ID NO: 16.

48. The method of any one of embodiments 1-47, wherein the CD3 binding domain comprises a second VH comprising an amino acid sequence having at least 98% identity to the amino acid sequence of SEQ ID NO: 15, and comprises a second VL comprising an amino acid sequence having at least 98% identity to the amino acid sequence of SEQ ID NO: 16.

49. The method of any one of embodiments 1-48, wherein the CD3 binding domain comprises a second VH comprising the amino acid sequence of SEQ ID NO: 15, and comprises a second VL comprising the amino acid sequence of SEQ ID NO: 16.

50. The method of any one of embodiments 46-49, wherein the second VH and the second VL are linked by a polypeptide linker.

51. The method of embodiment 50, wherein the polypeptide linker comprises an amino acid sequence of SEQ ID NO: 20.

52. The method of any one of embodiments 1-51, wherein the CD3 binding domain comprises an amino acid sequence having at least 90% identity to the amino acid sequence of SEQ ID NO: 19.

53. The method of any one of embodiments 1-52, wherein the CD3 binding domain comprises an amino acid sequence having at least 95% identity to the amino acid sequence of SEQ ID NO: 19.

54. The method of any one of embodiments 1-53, wherein the CD3 binding domain comprises an amino acid sequence having at least 98% identity to the amino acid sequence of SEQ ID NO: 19.

55. The method of any one of embodiments 1-54, wherein the CD3 binding domain comprises the amino acid sequence of SEQ ID NO: 19.

56. The method of any one of embodiments 1-55, wherein the bispecific antibody comprising a CLDN6 binding domain and a CD3 binding domain is a Fab-Fc-scFv.

57. The method of any one of embodiments 1-55, wherein the bispecific antibody comprising a CLDN6 binding domain and a CD3 binding domain is a IgG-scFv.

58. The method of any one of embodiments 1-55, wherein the bispecific antibody comprising a CLDN6 binding domain and a CD3 binding domain is a IgG-(scFv)₂.

59. The method of any one of embodiments 1-58, wherein the cancer is a solid tumor.

60. The method of embodiment 59, wherein the solid tumor expresses CLDN6.

61. The method of embodiment 60, wherein CLDN6 is expressed in at least 10% of the solid tumor cells.

62. The method of any one of embodiments 1-61, wherein the cancer is selected from ovarian cancer, testicular cancer, or endometrial cancer.

63. The method of embodiment 62, wherein the cancer is ovarian cancer.

64. The method of embodiment 62 or 63, wherein the ovarian cancer is platinum-refractory/resistant ovarian cancer (PRROC).

65. The method of embodiment 62, wherein the cancer is testicular cancer. 66. The method of embodiment 62, wherein the cancer is endometrial cancer. 67. A method of treating a cancer in a subject in need thereof, comprising:
intravenously administering to the subject a first dose of a bispecific antibody, wherein the first dose is selected from the group consisting of: about 22.5 μg, about 70 μg, about 140 μg, about 200 μg, about 280 μg, about 400 μg, about 560 μg, about 800 μg, or about 1000 μg, and
intravenously administering to the subject one or more subsequent doses, wherein each subsequent dose is selected from the group consisting of: about 22.5 μg, about 70 μg, about 140 μg, about 200 μg, about 280 μg, about 400 μg, about 560 μg, about 800 μg, about 1120 μg, about 1600 μg, about 2240 μg, about 3200 μg, about 4480 μg, about 6400 μg, about 8960 μg, or about 12800 μg,
wherein the bispecific antibody comprises a CLDN6 binding domain and a CD3 binding domain;
wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and
wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

68. The method of embodiment 67, wherein the first dose is about 22.5 μg.

69. The method of embodiment 67, wherein the first dose is about 70 μg.

70. The method of embodiment 67, wherein the first dose is about 140 μg.

71. The method of embodiment 67, wherein the first dose is about 200 μg.

72. The method of embodiment 67, wherein the first dose is about 280 μg.

73. The method of embodiment 67, wherein the first dose is about 400 μg.

74. The method of embodiment 67, wherein the first dose is about 560 μg.

75. The method of embodiment 67, wherein the first dose is about 800 μg.

76. The method of embodiment 67, wherein the first dose is about 1000 μg.

77. The method of any one of embodiments 67-76, wherein the one or more subsequent doses are in the same amount as the first dose.

78. The method of any one of embodiments 67-76, wherein the one or more subsequence doses are in a different amount as the first dose.

79. The method of any one of embodiments 67-78, wherein at least one or more of the one or more subsequent doses is about 22.5 μg.

80. The method of any one of embodiments 67-78, wherein at least one or more of the one or more subsequent doses is about 70 μg.

81. The method of any one of embodiments 67-78, wherein at least one or more of the one or more subsequent doses is about 140 μg.

82. The method of any one of embodiments 67-78, wherein at least one or more of the one or more subsequent doses is about 200 μg.

83. The method of any one of embodiments 67-78, wherein at least one or more of the one or more subsequent doses is about 280 μg.

84. The method of any one of embodiments 67-78, wherein at least one or more of the one or more subsequent doses is about 400 μg.

85. The method of any one of embodiments 67-78, wherein at least one or more of the one or more subsequent doses is about 560 μg.

86. The method of any one of embodiments 67-78, wherein at least one or more of the one or more subsequent doses is about 800 μg.

87. The method of any one of embodiments 67-78, wherein at least one or more of the one or more subsequent doses is about 1120 μg.

88. The method of any one of embodiments 67-78, wherein at least one or more of the one or more subsequent doses is about 1600 μg.

89. The method of any one of embodiments 67-78, wherein at least one or more of the one or more subsequent doses is about 2240 μg.

90. The method of any one of embodiments 67-78, wherein at least one or more of the one or more subsequent doses is about 3200 μg.

91. The method of any one of embodiments 67-78, wherein at least one or more of the one or more subsequent doses is about 4480 μg.

92. The method of any one of embodiments 67-78, wherein at least one or more of the one or more subsequent doses is about 6400 μg.

93. The method of any one of embodiments 67-78, wherein at least one or more of the one or more subsequent doses is about 8960 μg.

94. The method of any one of embodiments 67-78, wherein at least one or more of the one or more subsequent doses is about 12800 μg.

95. The method of embodiment 67, wherein the first dose is about 22.5 μg and at least one or more of the one or more subsequent doses is about 22.5 μg.

96. The method of embodiment 67, wherein the first dose is about 22.5 μg and at least one or more of the one or more subsequent doses is about 70 μg.

97. The method of embodiment 67, wherein the first dose is about 70 μg and at least one or more of the one or more subsequent doses is about 140 μg.

98. The method of embodiment 67, wherein the first dose is about 70 μg and at least one or more of the one or more subsequent doses is about 200 μg.

99. The method of embodiment 67, wherein the first dose is about 140 μg and at least one or more of the one or more subsequent doses is about 280 μg.

100. The method of embodiment 67, wherein the first dose is about 200 μg and at least one or more of the one or more subsequent doses is about 400 μg.

101. The method of embodiment 67, wherein the first dose is about 280 μg and at least one or more of the one or more subsequent doses is about 560 μg.

102. The method of embodiment 67, wherein the first dose is about 400 μg and at least one or more of the one or more subsequent doses is about 800 μg.

103. The method of embodiment 67, wherein the first dose is about 560 μg and at least one or more of the one or more subsequent doses is about 1120 μg.

104. The method of embodiment 67, wherein the first dose is about 800 μg and at least one or more of the one or more subsequent doses is about 1600 μg.

105. The method of embodiment 67, wherein the first dose is about 1000 μg and at least one or more of the one or more subsequent doses is about 2240 μg.

106. The method of embodiment 67, wherein the first dose is about 1000 μg and at least one or more of the one or more subsequent doses is about 3200 μg.

107. The method of embodiment 67, wherein the first dose is about 1000 μg and at least one or more of the one or more subsequent doses is about 4480 μg.

108. The method of embodiment 67, wherein the first dose is about 1000 μg and at least one or more of the one or more subsequent doses is about 6400 μg.

109. The method of embodiment 67, wherein the first dose is about 1000 μg and at least one or more of the one or more subsequent doses is about 8960 μg.

110. The method of embodiment 67, wherein the first dose is about 1000 μg and at least one or more of the one or more subsequent doses is about 12800 μg.

111. The method of any one of embodiments 67-110, wherein at least one subsequent dose of the one or more subsequent doses is administered one, two, three, four, five, six, seven, eight, nine, ten, eleven, twelve, or more than twelve weeks after the first dose.

112. The method of any one of embodiments 67-111, wherein at least one, two, three, four, five, six, seven, eight, nine, ten, eleven, twelve, or more than twelve subsequent doses are administered to the subject.

113. The method of any one of embodiments 67-112, wherein each subsequent dose is administered one week after a previous dose.

114. The method of any one of embodiments 67-113, wherein a second dose is administered one week after the first dose.

115. The method of any one of embodiments 67-114, wherein a second dose is administered two weeks after the first dose.

116. The method of any one of embodiments 67-115, wherein starting with a third dose, each subsequent dose is administered one week after a previous dose.

117. The method of any one of embodiments 67-116, wherein the bispecific antibody is administered over a period of about 10 minutes to about 30 minutes, about 10 minutes to about 20 minutes, or about 12 minutes to about 18 minutes.

118. The method of any one of embodiments 67-117, wherein the bispecific antibody is administered as part of a pharmaceutically acceptable composition comprising the bispecific antibody, a pharmaceutically acceptable buffer, a sugar, and a non-ionic surfactant.

119. The method of any one of embodiments 67-118, wherein the CLDN6 binding domain comprises a first VH comprising an amino acid sequence having at least 90% identity to the amino acid sequence of SEQ ID NO: 7, and comprises a first VL comprising an amino acid sequence having at least 90% identity to the amino acid sequence of SEQ ID NO: 8.

120. The method of any one of embodiments 67-119, wherein the CLDN6 binding domain comprises a first VH comprising an amino acid sequence having at least 95% identity to the amino acid sequence of SEQ ID NO: 7, and comprises a first VL comprising an amino acid sequence having at least 95% identity to the amino acid sequence of SEQ ID NO: 8.

121. The method of any one of embodiments 67-120, wherein the CLDN6 binding domain comprises a first VH comprising an amino acid sequence having at least 98% identity to the amino acid sequence of SEQ ID NO: 7, and comprises a first VL comprising an amino acid sequence having at least 98% identity to the amino acid sequence of SEQ ID NO: 8.

122. The method of any one of embodiments 67-121, wherein the CLDN6 binding domain comprises a first VH comprising the amino acid sequence of SEQ ID NO: 7, and comprises a first VL comprising the amino acid sequence of SEQ ID NO: 8.

123. The method of any one of embodiments 67-122, wherein the CLDN6 binding domain comprises a heavy chain (HC) and a light chain (LC), wherein the HC comprises an amino acid sequence having at least 90% identity to the amino acid sequence of SEQ ID NO: 17 and the LC comprises an amino acid sequence having at least 90% identity to the amino acid sequence of SEQ ID NO: 18.

124. The method of any one of embodiments 67-123, wherein the CLDN6 binding domain comprises a HC and a LC, wherein the HC comprises an amino acid sequence having at least 95% identity to the amino acid sequence of SEQ ID NO: 17 and the LC comprises an amino acid sequence having at least 95% identity to the amino acid sequence of SEQ ID NO: 18.

125. The method of any one of embodiments 67-124, wherein the CLDN6 binding domain comprises a HC and a LC, wherein the HC comprises an amino acid sequence having at least 98% identity to the amino acid sequence of SEQ ID NO: 17 and the LC comprises an amino acid sequence having at least 98% identity to the amino acid sequence of SEQ ID NO: 18.

126. The method of any one of embodiments 67-125, wherein the CLDN6 binding domain comprises a HC and a LC, wherein the HC comprises the amino acid sequence of SEQ ID NO: 17 and the LC comprises the amino acid sequence of SEQ ID NO: 18.

127. The method of any one of embodiments 67-126, wherein the CD3 binding domain comprises a second VH comprising an amino acid sequence having at least 90% identity to the amino acid sequence of SEQ ID NO: 15, and comprises a second VL comprising an amino acid sequence having at least 90% identity to the amino acid sequence of SEQ ID NO: 16.

128. The method of any one of embodiments 67-127, wherein the CD3 binding domain comprises a second VH comprising an amino acid sequence having at least 95% identity to the amino acid sequence of SEQ ID NO: 15, and comprises a second VL comprising an amino acid sequence having at least 95% identity to the amino acid sequence of SEQ ID NO: 16.

129. The method of any one of embodiments 67-128, wherein the CD3 binding domain comprises a second VH comprising an amino acid sequence having at least 98% identity to the amino acid sequence of SEQ ID NO: 15, and comprises a second VL comprising an amino acid sequence having at least 98% identity to the amino acid sequence of SEQ ID NO: 16.

130. The method of any one of embodiments 67-129, wherein the CD3 binding domain comprises a second VH comprising the amino acid sequence of SEQ ID NO: 15, and comprises a second VL comprising the amino acid sequence of SEQ ID NO: 16.

131. The method of any one of embodiments 127-130, wherein the second VH and the second VL are linked by a polypeptide linker.

132. The method of embodiment 131, wherein the polypeptide linker comprises an amino acid sequence of SEQ ID NO: 20.

133. The method of any one of embodiments 67-132, wherein the CD3 binding domain comprises an amino acid sequence having at least 90% identity to the amino acid sequence of SEQ ID NO: 19.

134. The method of any one of embodiments 67-133, wherein the CD3 binding domain comprises an amino acid sequence having at least 95% identity to the amino acid sequence of SEQ ID NO: 19.

135. The method of any one of embodiments 67-134, wherein the CD3 binding domain comprises an amino acid sequence having at least 98% identity to the amino acid sequence of SEQ ID NO: 19.

136. The method of any one of embodiments 67-135, wherein the CD3 binding domain comprises the amino acid sequence of SEQ ID NO: 19.

137. The method of any one of embodiments 67-136, wherein the bispecific antibody comprising a CLDN6 binding domain and a CD3 binding domain is a Fab-Fc-scFv.

138. The method of any one of embodiments 67-136, wherein the bispecific antibody comprising a CLDN6 binding domain and a CD3 binding domain is an IgG-scFv.

139. The method of any one of embodiments 67-136, wherein the bispecific antibody comprising a CLDN6 binding domain and a CD3 binding domain is an IgG-(scFv)₂.

140. The method of any one of embodiments 67-139, wherein the cancer is a solid tumor.

141. The method of embodiment 140, wherein the solid tumor expresses CLDN6.

142. The method of embodiment 141, wherein CLDN6 is expressed in at least 10% of the solid tumor cells.

143. The method of any one of embodiments 67-142, wherein the cancer is selected from ovarian cancer, testicular cancer, or endometrial cancer.

144. The method of embodiment 143, wherein the cancer is ovarian cancer.

145. The method of embodiment 143 or 144, wherein the ovarian cancer is platinum-refractory/resistant ovarian cancer (PRROC).

146. The method of embodiment 143, wherein the cancer is testicular cancer.

147. The method of embodiment 143, wherein the cancer is endometrial cancer.

148. A method of treating platinum refractory/resistant ovarian cancer (PRROC) in a subject in need thereof, comprising:
intravenously administering to the subject a first dose of a bispecific antibody, wherein the first dose is selected from the group consisting of: about 22.5 μg, about 70 μg, about 140 μg, about 200 μg, about 280 μg, about 400 μg, about 560 μg, about 800 μg, or about 1000 μg, and
intravenously administering to the subject one or more subsequent doses, wherein each subsequent dose is selected from the group consisting of: about 22.5 μg, about 70 μg, about 140 μg, about 200 μg, about 280 μg, about 400 μg, about 560 μg, about 800 μg, about 1120 μg, about 1600 μg, about 2240 μg, about 3200 μg, about 4480 μg, about 6400 μg, about 8960 μg, or about 12800 μg,
wherein the bispecific antibody comprises a CLDN6 binding domain and a CD3 binding domain;
wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and
wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

149. The method of embodiment 148, wherein the first dose is about 22.5 μg and at least one or more of the one or more subsequent doses is about 22.5 μg.

150. The method of embodiment 148, wherein the first dose is about 22.5 μg and at least one or more of the one or more subsequent doses is about 70 μg.

151. The method of embodiment 148, wherein the first dose is about 70 μg and at least one or more of the one or more subsequent doses is about 140 μg.

152. The method of embodiment 148, wherein the first dose is about 70 μg and at least one or more of the one or more subsequent doses is about 200 μg.

153. The method of embodiment 148, wherein the first dose is about 140 μg and at least one or more of the one or more subsequent doses is about 280 μg.

154. The method of embodiment 148, wherein the first dose is about 200 μg and at least one or more of the one or more subsequent doses is about 400 μg.

155. The method of embodiment 148, wherein the first dose is about 280 μg and at least one or more of the one or more subsequent doses is about 560 μg.

156. The method of embodiment 148, wherein the first dose is about 400 μg and at least one or more of the one or more subsequent doses is about 800 μg.

157. The method of embodiment 148, wherein the first dose is about 560 μg and at least one or more of the one or more subsequent doses is about 1120 μg.

158. The method of embodiment 148, wherein the first dose is about 800 μg and at least one or more of the one or more subsequent doses is about 1600 μg.

159. The method of embodiment 148, wherein the first dose is about 1000 μg and at least one or more of the one or more subsequent doses is about 2240 μg.

160. The method of embodiment 148, wherein the first dose is about 1000 μg and at least one or more of the one or more subsequent doses is about 3200 μg.

161. The method of embodiment 148, wherein the first dose is about 1000 μg and at least one or more of the one or more subsequent doses is about 4480 μg.

162. The method of embodiment 148, wherein the first dose is about 1000 μg and at least one or more of the one or more subsequent doses is about 6400 μg.

163. The method of embodiment 148, wherein the first dose is about 1000 μg and at least one or more of the one or more subsequent doses is about 8960 μg.

164. The method of embodiment 148, wherein the first dose is about 1000 μg and at least one or more of the one or more subsequent doses is about 12800 μg.

165. The method of any one of embodiments 148-165, wherein at least one, two, three, four, five, six, seven, eight, nine, ten, eleven, twelve, or more than twelve subsequent doses are administered to the subject.

166. The method of any one of embodiments 148-165, wherein each subsequent dose is administered one week after a previous dose.

167. The method of any one of embodiments 148-166, wherein a second dose is administered one week after the first dose.

168. The method of any one of embodiments 148-166, wherein a second dose is administered two weeks after the first dose.

169. The method of any one of embodiments 148-168, wherein starting with a third dose, each subsequent dose is administered one week after a previous dose.

170. The method of any one of embodiments 148-169, wherein the bispecific antibody is administered over a period of about 10 minutes to about 30 minutes, about 10 minutes to about 20 minutes, or about 12 minutes to about 18 minutes.

171. The method of any one of embodiments 148-170, wherein the bispecific antibody is administered as part of a pharmaceutically acceptable composition comprising the bispecific antibody, a pharmaceutically acceptable buffer, a sugar, and a non-ionic surfactant.

172. The method of any one of embodiments 148-171, wherein the CLDN6 binding domain comprises a first VH comprising an amino acid sequence having at least 95% identity to the amino acid sequence of SEQ ID NO: 7, and comprises a first VL comprising an amino acid sequence having at least 95% identity to the amino acid sequence of SEQ ID NO: 8.

173. The method of any one of embodiments 148-172, wherein the CLDN6 binding domain comprises a first VH comprising the amino acid sequence of SEQ ID NO: 7, and comprises a first VL comprising the amino acid sequence of SEQ ID NO: 8.

174. The method of any one of embodiments 148-173, wherein the CLDN6 binding domain comprises a HC and a LC, wherein the HC comprises an amino acid sequence having at least 95% identity to the amino acid sequence of SEQ ID NO: 17 and the LC comprises an amino acid sequence having at least 95% identity to the amino acid sequence of SEQ ID NO: 18.

175. The method of any one of embodiments 148-174, wherein the CLDN6 binding domain comprises a HC and a LC, wherein the HC comprises the amino acid sequence of SEQ ID NO: 17 and the LC comprises the amino acid sequence of SEQ ID NO: 18.

176. The method of any one of embodiments 148-175, wherein the CD3 binding domain comprises a second VH comprising an amino acid sequence having at least 95% identity to the amino acid sequence of SEQ ID NO: 15, and comprises a second VL comprising an amino acid sequence having at least 95% identity to the amino acid sequence of SEQ ID NO: 16.

177. The method of any one of embodiments 148-176, wherein the CD3 binding domain comprises a second VH comprising the amino acid sequence of SEQ ID NO: 15, and comprises a second VL comprising the amino acid sequence of SEQ ID NO: 16.

178. The method of any one of embodiments 148-177, wherein the second VH and the second VL are linked by a polypeptide linker.

179. The method of embodiment 178, wherein the polypeptide linker comprises an amino acid sequence of SEQ ID NO: 20.

180. The method of any one of embodiments 148-179, wherein the CD3 binding domain comprises an amino acid sequence having at least 95% identity to the amino acid sequence of SEQ ID NO: 19.

181. The method of any one of embodiments 148-180, wherein the CD3 binding domain comprises the amino acid sequence of SEQ ID NO: 19.

182. The method of any one of embodiments 148-181, wherein the bispecific antibody comprising a CLDN6 binding domain and a CD3 binding domain is a Fab-Fc-scFv.

183. A method of treating a cancer in a subject in need thereof, comprising:
intravenously administering to the subject a first dose of a bispecific antibody, wherein the first dose is selected from the group consisting of: about 22.5 μg, about 70 μg, about 140 μg, about 200 μg, about 280 μg, about 400 μg, about 560 μg, about 800 μg, or about 1000 μg, and
intravenously administering to the subject one or more subsequent doses, wherein each subsequent dose is selected from the group consisting of: about 22.5 μg, about 70 μg, about 140 μg, about 200 μg, about 280 μg, about 400 μg, about 560 μg, about 800 μg, about 1120 μg, about 1600 μg, about 2240 μg, about 3200 μg, about 4480 μg, about 6400 μg, about 8960 μg, or about 12800 μg,
wherein the bispecific antibody binds to claudin 6 (CLDN6) and CD3, the bispecific antibody comprising:
a first polypeptide comprising a first light chain comprising a first variable light chain region, wherein the first variable light chain region comprises:
   (1) a CDR1 sequence comprising the amino acid sequence of SEQ ID NO: 4;
   (2) a CDR2 sequence comprising the amino acid sequence of SEQ ID NO: 5; and
   (3) a CDR3 sequence comprising the amino acid sequence of SEQ ID NO: 6;
a second polypeptide comprising a first heavy chain comprising a first variable heavy chain region and a constant domain, wherein the first variable heavy chain region comprises:
   (1) a CDR1 comprising the amino acid sequence of SEQ ID NO: 1;
   (2) a CDR2 comprising the amino acid sequence of SEQ ID NO: 2, and
   (3) a CDR3 comprising the amino acid sequence of SEQ ID NO: 3; and
a third polypeptide comprising a second light chain and a second heavy chain and a constant domain, wherein:
   the second heavy chain comprises a second variable heavy chain region comprising:
   (1) a CDR1 comprising the amino acid sequence of SEQ ID NO: 9;
   (2) a CDR2 comprising the amino acid sequence of SEQ ID NO: 10; and
   (3) a CDR3 comprising the amino acid sequence of SEQ ID NO: 11; and
   the second light chain comprises a second variable light chain region comprising:
   (1) a CDR1 comprising the amino sequence of SEQ ID NO: 12;
   (2) a CDR2 comprising the amino acid sequence of SEQ ID NO: 13; and
   (3) a CDR3 comprising the amino acid sequence of SEQ ID NO: 14, or a variant thereof,
wherein:
   the first polypeptide comprises an amino acid sequence that is at least 95% identical to the amino acid sequence of SEQ ID NO: 18;
   the second polypeptide comprises an amino acid sequence that is at least 95% identical to the amino acid sequence of SEQ ID NO: 17; and
   the third polypeptide comprises an amino acid sequence that is at least 95% identical to the amino acid sequence of SEQ ID NO: 19.

184. The method of embodiment 183, wherein:
the first polypeptide of the bispecific antibody comprises the amino acid sequence of SEQ ID NO: 18;
the second polypeptide of the bispecific antibody comprises the amino acid sequence of SEQ ID NO: 17; and
the third polypeptide of the bispecific antibody comprises the amino acid sequence of SEQ ID NO: 19.

185. The method of embodiment 183 or 184, wherein the first dose is about 22.5 μg and at least one or more of the one or more subsequent doses is about 22.5 μg.

186. The method of embodiment 183 or 184, wherein the first dose is about 22.5 μg and at least one or more of the one or more subsequent doses is about 70 μg.

187. The method of embodiment 183 or 184, wherein the first dose is about 70 μg and at least one or more of the one or more subsequent doses is about 140 μg.

188. The method of embodiment 183 or 184, wherein the first dose is about 70 μg and at least one or more of the one or more subsequent doses is about 200 μg.

189. The method of embodiment 183 or 184, wherein the first dose is about 140 μg and at least one or more of the one or more subsequent doses is about 280 μg.

190. The method of embodiment 183 or 184, wherein the first dose is about 200 μg and at least one or more of the one or more subsequent doses is about 400 μg.

191. The method of embodiment 183 or 184, wherein the first dose is about 280 μg and at least one or more of the one or more subsequent doses is about 560 μg.

192. The method of embodiment 183 or 184, wherein the first dose is about 400 μg and at least one or more of the one or more subsequent doses is about 800 μg.

193. The method of embodiment 183 or 184, wherein the first dose is about 560 μg and at least one or more of the one or more subsequent doses is about 1120 μg.

194. The method of embodiment 183 or 184, wherein the first dose is about 800 μg and at least one or more of the one or more subsequent doses is about 1600 μg.

195. The method of embodiment 183 or 184, wherein the first dose is about 1000 μg and at least one or more of the one or more subsequent doses is about 2240 μg.

196. The method of embodiment 183 or 184, wherein the first dose is about 1000 μg and at least one or more of the one or more subsequent doses is about 3200 μg.

197. The method of embodiment 183 or 184, wherein the first dose is about 1000 μg and at least one or more of the one or more subsequent doses is about 4480 μg.

198. The method of embodiment 183 or 184, wherein the first dose is about 1000 μg and at least one or more of the one or more subsequent doses is about 6400 μg.

199. The method of embodiment 183 or 184, wherein the first dose is about 1000 μg and at least one or more of the one or more subsequent doses is about 8960 μg.

200. The method of embodiment 183 or 184, wherein the first dose is about 1000 μg and at least one or more of the one or more subsequent doses is about 12800 μg.

201. The method of any one of embodiments 183-200, wherein at least one, two, three, four, five, six, seven, eight, nine, ten, eleven, twelve, or more than twelve subsequent doses are administered to the subject.

202. The method of any one of embodiments 183-201, wherein each subsequent dose is administered one week after a previous dose.

203. The method of any one of embodiments 183-202, wherein a second dose is administered one week after the first dose.

204. The method of any one of embodiments 183-202, wherein a second dose is administered two weeks after the first dose.

205. The method of any one of embodiments 183-204, wherein starting with a third dose, each subsequent dose is administered one week after a previous dose.

206. The method of any one of embodiments 183-205, wherein the bispecific antibody is administered over a period of about 10 minutes to about 30 minutes, about 10 minutes to about 20 minutes, or about 12 minutes to about 18 minutes.

207. The method of any one of embodiments 183-206, wherein the bispecific antibody is administered as part of a pharmaceutically acceptable composition comprising the bispecific antibody, a pharmaceutically acceptable buffer, a sugar, and a non-ionic surfactant.

208. The method of any one of embodiments 183-207, wherein the cancer is a solid tumor.

209. The method of embodiment 208, wherein the solid tumor expresses CLDN6.

210. The method of embodiment 209, wherein CLDN6 is expressed in at least 10% of the solid tumor cells.

211. The method of any one of embodiments 183-210, wherein the cancer is selected from ovarian cancer, testicular cancer, or endometrial cancer.

212. The method of embodiment 211, wherein the cancer is ovarian cancer.

213. The method of embodiment 211 or 212, wherein the ovarian cancer is platinum-refractory/resistant ovarian cancer (PRROC).

214. The method of embodiment 211, wherein the cancer is testicular cancer.

215. The method of embodiment 211, wherein the cancer is endometrial cancer.

216. The method of any one of embodiments 1-215, wherein administration of the first dose of the bispecific antibody and administration of the one or more subsequent doses of the bispecific antibody results in a Cₘₐₓ approximately equivalent to 0.04 nM.

217. The method of any one of embodiments 1-215, wherein administration of the first dose of the bispecific antibody and administration of the one or more subsequent doses of the bispecific antibody results in a Cₘₐₓ of at least 0.04 nM.

218. The method of any one of embodiments 1-215, wherein administration of the first dose of the bispecific antibody and administration of the one or more subsequent doses of the bispecific antibody results in an area under the curve (AUC) of approximately 175 hr*ng/mL.

219. The method of any one of embodiments 1-215, wherein administration of the first dose of the bispecific antibody and administration of the one or more subsequent doses of the bispecific antibody results in an area under the curve (AUC) of at least 175 hr*ng/mL.

220. The method of any one of embodiments 1-215, wherein administration of the first dose of the bispecific antibody and administration of the one or more subsequent doses of the bispecific antibody results in minimal accumulation of anti-drug antibodies.

221. The method of any one of embodiments 1-215, wherein administration of the first dose of the bispecific antibody and administration of the one or more subsequent doses of the bispecific antibody results in a complete response per RECIST Guidelines.

222. The method of any one of embodiments 1-215, wherein administration of the first dose of the bispecific antibody and administration of the one or more subsequent doses of the bispecific antibody results in a partial response per RECIST Guidelines.

223. The method of any one of embodiments 1-215, wherein administration of the first dose of the bispecific antibody and administration of the one or more subsequent doses of the bispecific antibody results in a stable disease response per RECIST Guidelines.

224. The method of any one of embodiments 1-215, wherein administration of the first dose of the bispecific antibody and administration of the one or more subsequent doses of the bispecific antibody results in a complete response per Immune RECIST Guidelines.

225. The method of any one of embodiments 1-215, wherein administration of the first dose of the bispecific antibody and administration of the one or more subsequent doses of the bispecific antibody results in a partial response per Immune RECIST Guidelines.

226. The method of any one of embodiments 1-215, wherein administration of the first dose of the bispecific antibody and administration of the one or more subsequent doses of the bispecific antibody results in a stable disease response per Immune RECIST Guidelines.

227. The method of any one of embodiments 1-215, wherein administration of the first dose of the bispecific antibody and administration of the one or more subsequent doses of the bispecific antibody results in an increase in levels of one or more cytokines in the subject selected from IL-2, IL-6, IL-8, IL-10, MCP1, IFNγ, TNFα, GM-CSF and CRP.

228. The method of any one of embodiments 1-215, wherein administration of the first dose of the bispecific antibody and administration of the one or more subsequent doses of the bispecific antibody results in an increase in levels of one or more of CD3+ T cells, CD4+ T cells, CD*+ T cells, CD69+ T cells, or Ki67+ T cells in the subject.

229. The method of any one of embodiments 1-215, wherein administration of the first dose of the bispecific antibody and administration of the one or more subsequent doses of the bispecific antibody results in an increase in levels of one or more of T cells, B cells, natural killer (NK) cells, monocytes, macrophages, neutrophils, eosinophils, basophils, mast cells, or dendritic cells in the subject.

230. The method of any one of embodiments 1-215, wherein administration of the first dose of the bispecific antibody and administration of the one or more subsequent doses of the bispecific antibody results in a decrease in CLDN6 expression in the tumor microenvironment.

231. The method of any one of embodiments 1-215, wherein administration of the first dose of the bispecific antibody and administration of the one or more subsequent doses of the bispecific antibody results in a change in the number and kind of infiltrating lymphocytes (TILs).

232. The method of any one of embodiments 1-215, wherein administration of the first dose of the bispecific antibody and administration of the one or more subsequent doses of the bispecific antibody results in a decrease in tumor size.

233. The method of any one of embodiments 1-215, wherein administration of the first dose of the bispecific antibody and administration of the one or more subsequent doses of the bispecific antibody results in depletion of tumor cells in the subject.

234. The method of any one of embodiments 1-215, wherein administration of the first dose of the bispecific antibody and administration of the one or more subsequent doses of the bispecific antibody results in an increased survival rate over a period of time as compared to subjects that do not receive the bispecific antibody.

### EXAMPLES

The following examples are illustrative, but not limiting, of the compounds, compositions, and methods described herein. Other suitable modifications and adaptations known to those skilled in the art are within the scope of the following embodiments.

### Example 1: Phase La Dose Escalation of CLDN6/CD3 Bispecific Antibody

Subjects with PRROC, testicular, or endometrial cancer may be enrolled in one of approximately 9 dose escalation cohorts to assess safety, tolerability and to determine the RDE. Study drug is to be administered once weekly (Q1W) for each cycle. A cycle is defined as 28 days. Subjects will be dosed until documentation of disease progression, unacceptable toxicity, or subject/physician decision.

The CLDN6/CD3 bispecific utilized in the present example is as provided for herein. Specifically, the CLDN6/CD3 bispecific referenced in the present example is of Fab-Fc-scFv format and is defined by a CLDN6 HC of SEQ ID NO: 17, a CLDN6 LC of SEQ ID NO: 18, and a CD3 scFv+constant domain of SEQ ID NO: 19.

The CLDN6/CD3 bispecific is formulated at 15 mg/mL as a sterile, iso-osmotic clear aqueous solution. It contains no preservatives and is intended for single-use parenteral administration only. The formulation includes 20 mM L-Histidine, 240 mM Sucrose, and 0.03% (w/v) Polysorbate 20. The pH is 6.0.

### Administration

Infusions are administered intravenously over 15 (±3) minutes using an infusion pump and syringe. If medically indicated, interruption in dose delivery, or slowing of infusion, is allowed.

### Premedications for the CLDN6/CD3 Bispecific

Prior to the priming dose and the first full dose in Cycle 1, subjects are pre-medicated with:
Corticosteroid (oral or iv dexamethasone, 16 mg or equivalent)
Antihistamines (oral or iv diphenhydramine, 50 mg or equivalent)

### Inclusion Criteria

### Inclusion Criteria (Subjects Must Meet all the Following Criteria):

1. Written informed consent provided prior to any screening procedures.
2. Male or female subjects, ≥18 years of age at the time of obtaining informed consent.
3. Ovarian cancer subjects with platinum-resistant (i.e., platinum-free interval <6 months) recurrent epithelial (non-mucinous) ovarian, fallopian tube, or primary peritoneal cancer who have progressed on or after standard of care therapies, including at least 1 line of therapy containing platinum derivative and taxane. Participant must have had prior treatment with bevacizumab or did not receive bevacizumab based on Investigator judgment. These subjects must have histologically or cytologically confirmed CLDN6-positive (defined as CLDN6 expression ≥10% of the tumor cells (≥+1 intensity) by immunohistochemistry [IHC] testing) advanced solid tumors previously treated with standard of care systemic therapy, or for whom no standard therapy is available.
4. Testicular germ cell cancer subjects with at least 2 prior lines of treatment, including platinum-based chemotherapy and either salvage chemotherapy or high dose chemotherapy with autologous stem cell transplant. These subjects are exempt from the requirement for a histologically or cytologically confirmed CLDN6-positive (by immunohistochemistry testing) tumor.
5. Endometrial carcinoma subjects with disease progression on or after treatment with at least 1 platinum-containing or immunotherapy-based regimen for advanced, recurrent, or metastatic disease. These subjects must have histologically or cytologically confirmed CLDN6-positive (defined as CLDN6 expression 10% of the tumor cells (≥+1 intensity) by immunohistochemistry [IHC] testing) advanced solid tumors previously treated with standard of care systemic therapy, or for whom no standard therapy is available.
   Note: Toxicity from prior therapy must be resolved to National Cancer institute (NCI) Common Terminology Criteria for Adverse Events (CTCAE) version 5.0 Grade ≤1, with the exception of alopecia and up to grade 2 peripheral neuropathy.
6. Willingness to provide archival formalin-fixed, paraffin-embedded (FFPE) tumor tissue (10-20 freshly cut slides) collected within the previous 2 years, when available. If no archival tissue is available that was collected within a 2-year timeframe, or if the minimum number of slides is not available, willingness to undergo a pretreatment core or excisional biopsy (fine needle aspirate [FNA] is not adequate) if medically feasible and safe. Note: this may be performed during pre-screening period.
7. Measurable disease per RECIST 1.1.
8. Eastern Cooperative Oncology Group (ECOG) performance status of 0 or 1 and life expectancy of 12 weeks.
9. Adequate organ function as defined by:
   a. Absolute neutrophil count (ANC) ≥1.5×10⁹/L (1000/μL).
   b. Platelets ≥100.0×10⁹/L (100 000/μL).
   c. Hemoglobin ≥8.5.0 g/dL.
   d. Creatinine clearance (CrCl) ≥50 mL/min as calculated by the Cockroft-Gault method.
   e. Potassium n=between LLN and 5.5 mmol/L. If the subject is asymptomatic and no intervention is indicated, levels between LLN and 3.0 mmol/L are acceptable.
   f. Sodium between 130 and 150 mmol/L.
   g. Calcium (corrected for albumin): total between 2.0 and 2.9 mmol/L and ionized calcium between 1.0 and 1.5 mmol/L.
   h. Magnesium between 0.5 and 1.23 mmol/L.
   i. Serum albumin ≥3.0 g/dL (≥30 g/L).
   j. Serum total bilirubin ≤1.5× the upper limit of normal (ULN).
   k. Aspartate aminotransferase (AST)≤3.0×ULN; alanine aminotransferase (ALT) ≤3.0×ULN.
10. Women of child-bearing potential (WCBP), defined as a sexually mature woman who has not undergone surgical sterilization or who has not been naturally postmenopausal for at least 12 consecutive months (i.e., who has had menses any time in the preceding 12 consecutive months) must agree to use 2 effective contraceptive methods; examples include oral, parenteral, or implantable hormonal contraceptive, intra-uterine device, barrier contraceptive with spermicide, partner's latex (or polyisoprene, if latex allergy) condom or vasectomy while on study treatment and for at least 4 months after the last dose of the study drug.
   a. WCBP must have a negative serum pregnancy test within 7 days prior to first dose of the study drug.
   b. Male subjects must agree to use a latex (or polyisoprene, if latex allergy) condom, even if they had a successful vasectomy, while on study treatment and for at least 12 weeks after the last dose of the study drug.
11. Subjects must be willing and able to sign the informed consent form, and to adhere to the study visit schedule and other protocol requirements.

### Exclusion Criteria

### Exclusion Criteria (Subjects Must not Meet any of the Following Criteria):

1. Evidence of central nervous system metastases, leptomeningeal disease, or spinal cord compression.
2. Prior history of malignancy other than inclusion diagnosis within 3 years prior to first dose of the study drug.
   Note: subjects with adequately treated basal cell or squamous cell skin cancer, non-invasive superficial bladder cancer, in situ cervical cancer, in situ breast cancer, in situ prostate cancer may be eligible if have shown no evidence of active disease for 2 years prior to first dose of drug.
3. Uncontrolled significant active infection or any medical or other condition that in the opinion of the investigator(s) would preclude the subject's participation in the study.
4. Major surgery (not including placement of vascular access device or tumor biopsies) within 4 weeks prior to first dose of study drug, or no recovery from side effects of such intervention.
5. Cardiovascular disease, e.g., stroke or myocardial infarction (MI) within 6 months of first dose of study drug, New York Heart Association (NYHA) class 3 or 4 congestive heart failure, unstable angina pectoris, serious cardiac arrythmias, etc.
6. Autoimmune disorders requiring chronic systemic therapy or any other form of immunosuppressive therapy while on the study. Recent or current use of inhaled steroids, physiological substitution in case of adrenal insufficiency, or local or topical use is not exclusionary.
7. Subjects with testicular germ cell cancer who have had less than 2 prior lines of treatment.
8. Prior CLDN6-targeted therapy.
9. Known or suspected allergy to the study drug or any component of the study drug.
10. Concurrent participation in another investigational clinical trial.
11. Pregnant or breast-feeding females.
12. Any other severe acute or chronic medical or psychiatric conditions or laboratory abnormality that may increase the risk associated with the study participation or the study drug administration or may interfere with the interpretation of study results and, in the judgment of the Investigator, would make the subject inappropriate for enrollment in this study.

### Trial Objectives and Endpoints

The objectives and corresponding endpoints for Phase 1a are provided in Table 13 below:

TABLE 13
Phase 1a Dose Escalation Objectives and Endpoints

| | |
|---|---|
| Objective | Endpoint |

Phase 1a, Dose Escalation

| | |
|---|---|
| Primary Objectives | Endpoints |
| To characterize the safety and tolerability | Incidence of dose limiting toxicities (DLTs). |
| of CLDN6/CD3 bispecific in subjects | Type, incidence, and severity of adverse events (AEs), |
| with PRROC and other advanced | serious adverse events (SAEs), adverse events of special |
| CLDN6- positive solid tumors (testicular | interest (AESI), and laboratory abnormalities using the |
| and endometrial). | National Cancer Institute (NCI) Common Terminology |
| | Criteria for Adverse Events (CTCAE) v.5.0. |
| | Frequency and duration of dose interruptions and |
| | reductions. |
| To establish the recommended doses for | Doses of CLDN6/CD3 bispecific in μg (IV) for the |
| expansion (RDE) of CLDN6/CD3 | treatment of subjects in Phase 1b. |
| bispecific. | |
| Secondary Objectives | Endpoints |
| To characterize the pharmacokinetic (PK) | PK parameters including, but not limited to maximum |
| profile of CLDN6/CD3 bispecific. | concentration (Cₘₐₓ), time to Cₘₐₓ (Tₘₐₓ), area under the |
| | curve (AUC), and terminal half-life (t_{1/2}). |
| To evaluate the immunogenicity of | Assessment of anti-drug antibodies (ADA). |
| CLDN6/CD3 bispecific. | |
| To evaluate preliminary efficacy of | Overall response rate (ORR), disease control rate (DCR) |
| CLDN6/CD3 bispecific according | based on best overall response (BOR), clinical benefit rate |
| to Response Evaluation Criteria in | (CBR), and duration of response (DOR) assessed by |
| Solid Tumors (RECIST v1.1). | Investigators according to Response Evaluation Criteria in |
| | Solid Tumors (RECIST v1.1). |
| To evaluate the efficacy of | iORR, iDCR, iCBR, iDOR, assessed by Investigators |
| CLDN6/CD3 bispecific as per Immune | according to iRECIST. |
| RECIST (iRECIST). | |
| Exploratory Objective | Endpoints |
| Assess changes in pro-inflammatory | Change over time of pro-inflammatory cytokines (e.g., |
| biomarkers. | TNFα, IFNγ, GM-CSF, IL-1, IL-2, IL6, CRP, etc.). |
| Evaluate the pharmacodynamic (PD) | Expression of markers of subset, activation, and |
| effect of CLDN6/CD3 bispecific | proliferation by T cells (e.g. CD3, CD4, CD8, CD69, Ki67, |
| monotherapy in modulating the immune | etc.). |
| system in blood. | Number and kind of immune cell subsets such as T cells in |
| | circulation. |
| Assess changes within the tumor and | Changes in CLDN6 expression. |
| microenvironment. | Change in the number and kind of infiltrating lymphocytes |
| | (TILs). |

Expansion Doses for Phase 1b will be determined by Sponsor in conjunction with SRC based upon all available safety, PK, PD, biomarker, and preliminary efficacy data from Phase 1a.

Accelerated Titration Cohorts (1-3): Cohorts 1-3 will have an accelerated titration design to enroll one subject each. For the one subject entering Cohort 1, if no ≥Grade 2 AEs occur, and upon SRC approval, Cohort 2 may then enroll a single subject. Assuming no ≥Grade 2 AEs in Cohorts 1 and 2, enrollment in Cohort 3 will proceed following this same paradigm. However, in the event of any ≥Grade 2 AE in any of the first three cohorts, enrollment will expand to 3 subjects per cohort by the standard 3+3 design. A nuance of this approach will be that a Grade 2 AE will not be considered a dose-limiting toxicity, rather a special measure taken to enhance safety of dosing. If a DLT occurs in the first subject enrolled in the cohort, the study will revert to a 3+3 design, and an additional 2 subjects will be enrolled to the same Cohort.

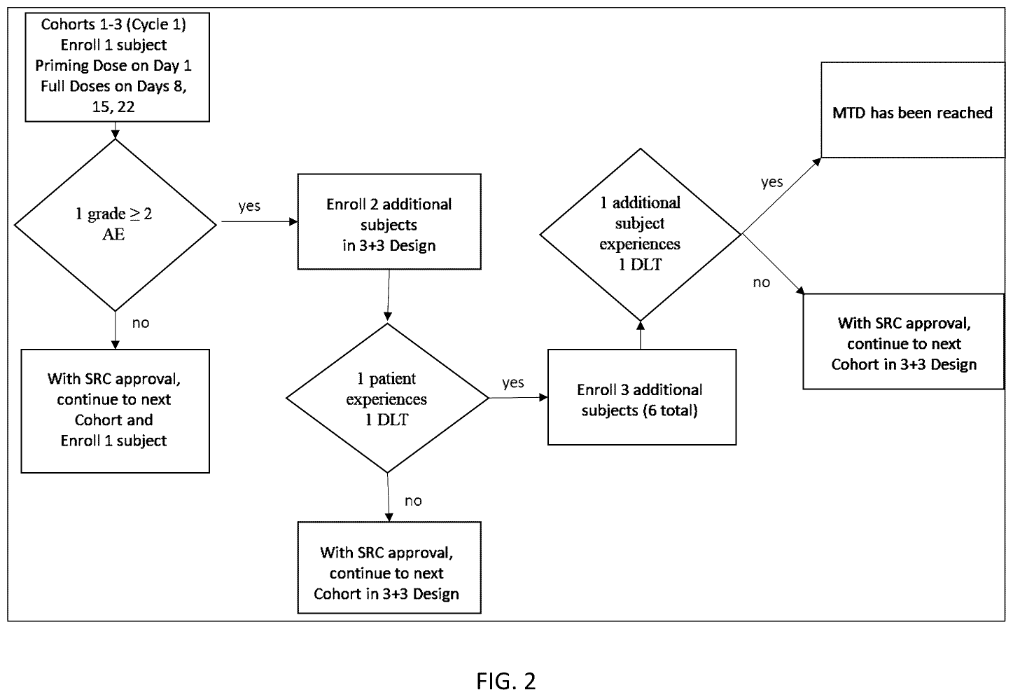

In the 3+3 design, 3 subjects are enrolled in each cohort, unless a dose-limiting toxic event occurs. A dose-limiting toxicity (DLT) is defined as any Grade ≥3 AE that occurs and is determined to be related to study product and meets the criteria defined above. If no DLT occurs in any of the subjects enrolled in a cohort during the 4-week DLT Observation Period, and upon review and approval of the SRC, dose escalation to the next Cohort will proceed. If a DLT occurs in one of the three subjects enrolled in the cohort, an additional 3 subjects will be enrolled in the same Cohort. If none of the 3 newly added subjects experiences a DLT, and upon review and approval of the SRC, dose escalation to the next Cohort will proceed. If ≥1 DLT occurs in any of the 3 newly added subjects (i.e. 2/6 subjects), the maximum tolerated dose (MTD) will have been considered attained. The enrollment for Cohorts 1-3 is as illustrated in FIG. 2.

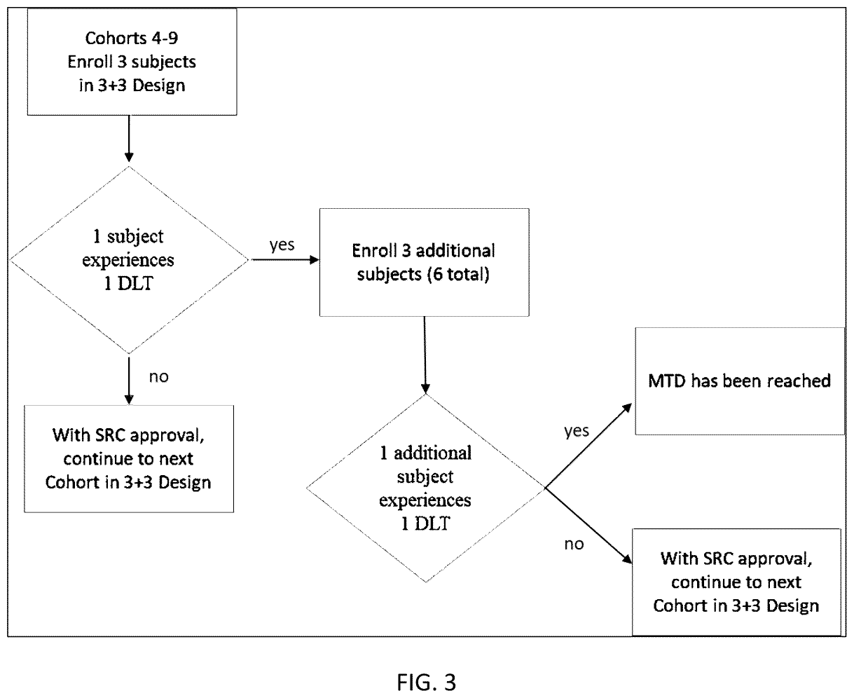

Dose Escalation Cohorts (4-9): After the first three dose level cohorts have established safety during the DLT Observation Period, Cohort 4 may begin, with enrollment proceeding using the standard 3+3 design. Cohorts 4-9 will enroll 3 subjects each unless a dose-limiting toxic event occurs. Dose-limiting toxicities (DLTs) are defined as any Grade ≥3 AE that occurs and is determined to be related to study product and meets the criteria as defined above. In Cohorts 4-9, subject enrollment will be staggered for safety. After the priming dose of the CLDN6/CD3 bispecific has been administered to the first subject enrolled in a cohort, there will be a two-week enrollment pause, to observe for any unexpected reactions or toxicities. No additional subjects will be enrolled in the study during this two-week period. If no DLT occurs in any of the 3 subjects in a cohort during the 4-week DLT Observation Period, and upon review and approval of the SRC, dose escalation to the next Cohort will proceed. If a DLT occurs in 1 subject, an additional 3 subjects will be enrolled to the same Cohort. If none of the 3 newly added subjects has DLT, upon review and approval of the SRC, dose escalation to the next Cohort will proceed. If ≥1 DLT occurs in any of the 3 newly added subjects (i.e. 2/6 subjects), the maximum tolerated dose (MTD) will have been considered attained. The enrollment for Cohorts 4-9 is as illustrated in FIG. 3.

All subjects in Phase 1a cohorts 1-9 will be admitted for in-subject monitoring for 24-hours following both their priming dose and their first full dose of the CLDN6/CD3 bispecific in cycle 1.

Although a classical 3+3 design is used, the decision to escalate the dose from one cohort to the next will take into account quantitative approaches, such as PK/PD modeling, as part of the assessment to support the optimization of dose to be selected for further development and leverage all available data including PK, PD, efficacy, and safety of the study drug. This approach will help select a pharmacologically optimal dose for further development rather than the maximum tolerated dose.

### Dosage, Administration, and Schedule

The cohort specified dose (see Table 14 below) of the CLDN6/CD3 bispecific will be administered as an IV infusion once weekly. Duration of all infusions is 15 minutes (±3 minutes). Please refer to the study specific pharmacy manual regarding full dosing and administration instructions.

The priming dose of CLDN6/CD3 bispecific will be administered on Cycle 1 Day 1 for all subjects. The full cohort dose will be administered on Day 8 and weekly thereafter.

TABLE 14
Provisional Dose Levels for Dose Escalation

| | | |
|---|---|---|
| | Priming Dose | Full Dose |
| Cohort | Cycle 1, Day 1 | Cycle 1, Day 8+ |

| | | | | |
|---|---|---|---|---|
| 1^{a} | 22.5 | μg | 22.5 | μg |
| 2^{a} | 22.5 | μg | 70 | μg |
| 3^{a,b} | 70 | μg | 200 | μg |
| 4 | 200 | μg | 400 | μg |
| 5 | 400 | μg | 800 | μg |
| 6 | 800 | μg | 1600 | μg |
| 7 | 1000 | μg | 3200 | μg |
| 8 | 1000 | μg | 6400 | μg |
| 9 | 1000 | μg | 12800 | μg |

^{a}An accelerated titration design, enrolling only 1 subject at dose level 1, with conversion to 3 + 3 design if the subject experienced any ≥ Grade 2 adverse event regardless of attribution, unless it can be clearly attributed to disease progression.
^{b}For Cohort 3, if there were no ≥ Grade 2 AEs in Cohorts 1 or 2, Cohort 3 will continue to follow the accelerated titration design of n = 1. Therefore Cohort 3 may enroll as few as 1, or as many as 6 subjects, prior to escalation to Cohort 4.

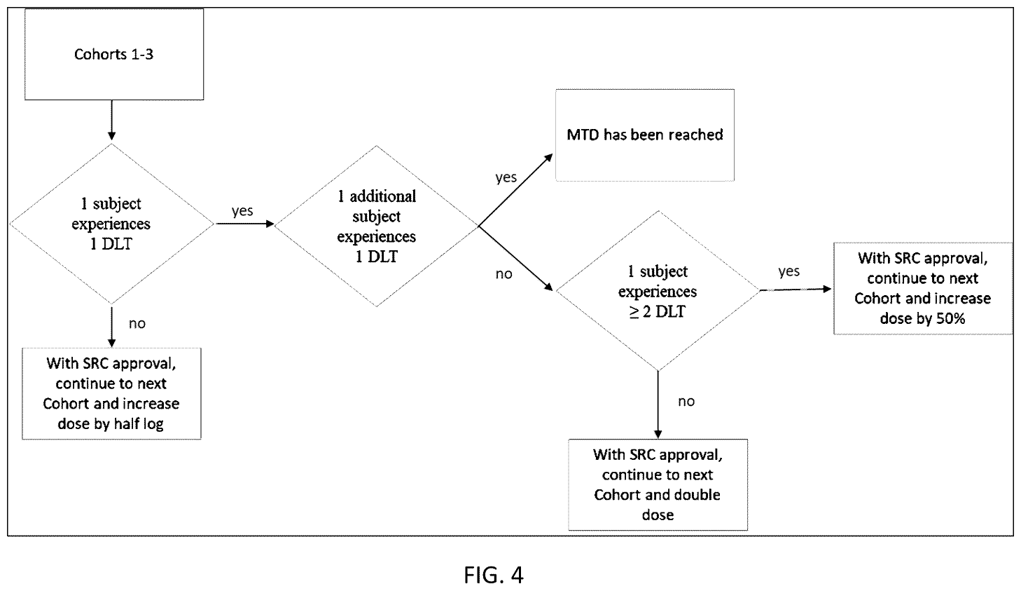

Cohorts 1-3: Cohort 1 will begin with a 22.5 μg priming dose, followed by once weekly doses of 22.5 μg (the calculated MABEL dose). The dose to be evaluated at cohorts 2 and 3 will be half log dose increases from the previous cohort dose unless a DLT is observed. If a single subject in a cohort experiences a DLT then the subsequent cohort dose will be double the current dose instead of a half log increase. If one subject experiences 2 DLTs during the DLT observation period, then the subsequent cohort dose will increase by 50%. If 2 subjects in a single cohort experience a DLT, then the MTD has been reached. The dose escalation rules for Cohorts 1-3 are as illustrated in FIG. 4.

After the first three cohorts have established safety during the DLT Observation Period, subjects may be enrolled in up to 6 additional cohorts (4-9), at doses that have cleared DLT safety assessment and been approved by the SRC, to further evaluate safety, PK, and preliminary efficacy, and to inform optimal selection of RDE.

Cohorts 4-9: In Cohorts 4-9, subject enrollment will be staggered for safety. After the priming dose of the CLDN6/CD3 bispecific is administered to the first subject enrolled in a cohort, there will be a two-week enrollment pause, to observe for any unexpected reactions or toxicities. No additional subjects will be enrolled in the study during this two-week period.

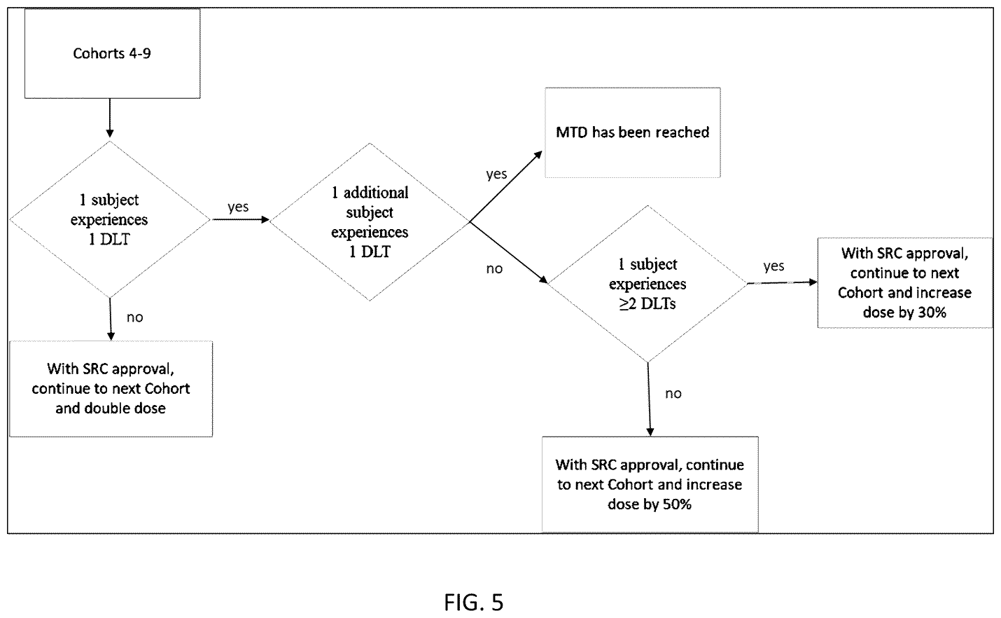

In cohort 4 and subsequent cohorts, the dose will be doubled from the previous cohort until a DLT is observed. If a single subject in a cohort experiences a DLT, then the subsequent cohort dose will increase by 50%. If one subject experiences 2 DLTs during the DLT observation period, then the subsequent cohort dose will increase by 30%. If 2 subjects in a single cohort experience a DLT, then the MTD has been reached. SRC will review all safety data, along with any available PK and PD data from the previous cohorts and approve continuation to the next dose level. The dose escalation rules for Cohorts 4-9 are as illustrated in FIG. 5.

Dose escalation will continue until the MTD is reached, or the RDE is otherwise determined before reaching the MTD. If the highest planned dose level is completed and determined to be safe and the MTD is not exceeded, the SRC will review all available study data and decide whether further dose escalation is justified. Intermediate dose levels, as well as alternative dosing schedules, may be added during the study.

The CLDN6/CD3 bispecific will be administered at the study site by a qualified staff member. A physician or appropriately qualified medical professional must be available at the time of administration. The date, time, package lot number, dose, and the start and stop time of infusion will be recorded on the eCRF. Please refer to the pharmacy manual for more details.

### Study Assessments

Assessment of Efficacy: At baseline and during the course of the clinical trial, tumor assessments will be performed as clinically indicated as per the Schedule of Assessments (Table 15 below). Results of each assessment should be entered in the eCRFs.

TABLE 15
Schedule of Assessments

| | Phase | | | | | | | | | | | | |
|---|---|---|---|---|---|---|---|---|---|---|---|---|---|
| | Screening | Treatment | | | | | | | | | | | |
| | Period | | | | | | | | | | | | |
| | | Cycle 1 | | | | Cycle 2 | | | | Cycle 3+ | | | |
| | Day in cycle | | | | | | | | | | | | |
| | | 1 | 8 | 15 | 22 | 1 | 8 | 15 | 22 | 1 | 8 | 15 | 22 |
| | Visit window [days] | | | | | | | | | | | | |
| | ≤28 days | 0 | +1 | +1 | +1 | +1 | +1 | +1 | +1 | +1 | +1 | +1 | +1 |
| Informed | X | | | | | | | | | | | | |
| consent | | | | | | | | | | | | | |
| Archival FFPE | X | | | | | | | | | | | | |
| tumor tissue | | | | | | | | | | | | | |
| sample | | | | | | | | | | | | | |
| collection^{c} | | | | | | | | | | | | | |
| Tumor Biopsy | X^{d} | | | | | | | X^{e} | | | | | |
| Treatment | | X^{f} | X | X | X | X | X | X | X | X | X | X | X |
| administration | | | | | | | | | | | | | |
| Eligibility | X | | | | | | | | | | | | |
| criteria | | | | | | | | | | | | | |
| Medical history | X | | | | | | | | | | | | |
| Concomitant | X | X | X | X | X | X | X | X | X | X | X | X | X |
| medications | | | | | | | | | | | | | |
| Adverse events | X | X | X | X | X | X | X | X | X | X | X | X | X |
| Physical | X | X | X | | | | | | | X | | | |
| examinations^{g} | | | | | | | | | | | | | |
| ECOG PS | X | X | | | | X | | | | X | | | |
| 12-lead ECG^{h} | X | X | | | | X | | | | X | | | |
| Bone scanⁱ | X | | | | | | | | | | | | |
| CT Scan/MRI^{j} | X | | | | | | | | | X | | | |
| Pregnancy | X | X | | | | X | | | | X | | | |
| testing^{k} | | | | | | | | | | | | | |
| CBC^{l} | X | X | X | | | X | | | | X | | | |
| CMP^{m, r} | X | X | X | | | X | X | | | X | X | | |
| Lipid panelⁿ | X | X | | | | X | | | | X | | | |
| Coagulation^{o} | X | X | | | | X | | | | X | | | |
| Urine testing | X | X | | | | X | | | | X | | | |
| dipstick^{p} | | | | | | | | | | | | | |
| Pre- | | X | X | | | | | | | | | | |
| treatment | | | | | | | | | | | | | |
| with | | | | | | | | | | | | | |
| diphenhydramine | | | | | | | | | | | | | |
| Pre- | | X | X | | | | | | | | | | |
| treatment | | | | | | | | | | | | | |
| with | | | | | | | | | | | | | |
| dexamethasone | | | | | | | | | | | | | |
| In-subject | | X | X | | | | | | | | | | |
| monitoring | | | | | | | | | | | | | |
| for 24- | | | | | | | | | | | | | |
| hours^{s} | | | | | | | | | | | | | |
| Anti-drug | | X | X | X | X | X | | | | X | | | |
| antibodies^{a} | | | | | | | | | | | | | |
| Pharmacokinetics | | See Table 17 and Table 18 for PK sampling timepoints | | | | | | | | | | | |
| Pharmacodynamics | | See Table 19 and Table 20 for PD sampling timepoints | | | | | | | | | | | |
| Survival | | | | | | | | | | | | | |
| Follow-up^{b} | | | | | | | | | | | | | |

| Phase | | | | |
|---|---|---|---|---|
| Follow-up | | | | |
| Period | | | | |
| Safety^{a} | | | Survival^{b} | |
| Day in cycle | | | | |
| 28 days | 90 days | | | Every 3 months |
| Visit window [days] | | | | |
| ±7 | | +14 | | ±1 |

| | | | |
|---|---|---|---|
| Informed consent | | | |
| Archival FFPE tumor | | | |
| tissue sample collection^{c} | | | |
| Tumor Biopsy | | | |
| Treatment administration | | | |
| Eligibility criteria | | | |
| Medical history | | | |
| Concomitant medications | X | X | |
| Adverse events | X | X | |
| Physical examinations^{g} | X | | |
| ECOG PS | X | | |
| 12-lead ECG^{h} | X | | |
| Bone scanⁱ | X | | |
| CT Scan/MRI^{j} | X | | |
| Pregnancy testing^{k} | X | | |
| CBC^{l} | X | | |
| CMP^{m, r} | X | | |
| Lipid panelⁿ | X | | |
| Coagulation^{o} | X | | |
| Urine testing dipstick^{p} | X | | |
| Pre-treatment with | | | |
| diphenhydramine | | | |
| Pre-treatment with | | | |
| dexamethasone | | | |
| In-subject monitoring for 24- | | | |
| hours^{s} | | | |
| Anti-drug antibodies^{q} | | | |
| Pharmacokinetics | | | |
| Pharmacodynamics | | | |
| Survival Follow-up^{b} | | | X |

^{a}EOT assessments will be conducted at Safety follow-up visit, approximately 28 days after the date of last treatment administration. All subjects will be followed for AEs for 28 days after the last treatment administration or until resolution or stabilization of all treatment-related AEs to either ≤Grade 2 or baseline, whichever is longer, or until the subject is lost to follow-up. At any time after 28 days from the last dose of study treatment, the Investigator may report any SAE that he/she believes is related to study treatment. Subjects will also be followed for 90 days after last treatment administration for monitoring and reporting late immune-related adverse events.
^{b}Subjects will be followed for safety for 90 days in Phase 1a and will be followed for survival in Phase 1b only.
^{c}Archival FFPE should be provided if fresh biopsy is not feasible for all subjects for CLDN6 testing. An archival tumor block or a minimum of 10 freshly cut unstained slides should be sent to the central lab.
^{d}FFPE tissue for immunohistochemistry testing of CLDN6 from a recently obtained core or excisional biopsy that is obtained after discontinuation of the most recent anticancer therapy is required for all subjects, if this is not feasible then archival FFPE should be obtained. Tumor biopsy (or archival tissue) will be required for all subjects in Phase 1a at screening, and also for subjects enrolled in the Phase 1b expansion phase in order to evaluate changes in CLDN6 expression and tumor infiltrating lymphocytes (TILs). A Formalin-Fixed, Paraffin-Embedded (FFPE) tumor biopsy (excisional or core needle) will be required during the screening period (archival tissue may be accepted if biopsy is not possible).
^{e}A second tumor biopsy (optional) will be requested at Week 8 (±1 week) after first dose of the CLDN6/CD3 bispecific, in order to evaluate changes in CLDN6 expression and biomarker analysis.
^{f}Priming dose to be administered cycle 1 day 1, the full dose will be administered on day 8 and weekly thereafter. For Phase 1a escalation doses see Table 14.
^{g}Physical examinations will include measurements of weight and vital signs (resting HR, BP, RR, and temperature). Height will be recorded at the baseline visit only. No need to repeat physical examinations on C1/D1 if the baseline physical examination was performed within ≤3 days.
^{h}A standard 12-lead ECG will be used in this study. Each ECG, both at Screening and during the study, is to be performed in triplicate approximately 2 minutes apart after the subject has been semi-recumbent for at least 5 minutes. Each of the ECG parameters (heart rate, QTcF interval QTcB interval, PR interval, and QRS duration), values from each of the triplicate measurements will be entered in the eCRF.
ⁱA bone scan using whole-body bone MRI, 99m-technetium-based bone scans, or 18F-sodium fluoride PET will be performed during Screening to establish a baseline (a bone scan performed per local SOC within 6 weeks before C1/D1 is acceptable), approximately every 24 weeks (in conjunction with a scheduled tumor assessment visit), and as clinically indicated. Lesions identified on bone scans should be followed with MRI or CT.
^{j}Tumor assessments will be performed at baseline and every 8 weeks (+1 week) after C1/D1 until radiological disease progression and will include: Chest: CT with contrast, Abdomen, pelvis, and other known or suspected sites of the disease: CT (with contrast) or MRI (with contrast) If imaging with contrast is contraindicated, imaging without contrast should be performed and reason for contraindication should be captured in the source documents. Tumor assessments will be performed using the same imaging modality and under the same operating conditions as the baseline scans. . If a subject discontinues the study for a reason other than disease progression and agrees to be followed for the specified time period in each phase of the study, tumor assessments will continue every 8 weeks (+1 week) until documentation of progression.
^{k}Only required for pre- and peri-menopausal women. If a serum test was performed within 7 days prior to C1/D1, the test does not need to be repeated. During the study, urine or serum testing should be at D1 of each Cycle. If a urine test is positive, serum testing should be performed for confirmation.
^{l}CBC (complete blood count) consisting of RBCs, Hb, Hct, MCV, MCH, WBC with 5-part differential and platelet count plus reticulocyte count. CBCs will be collected before treatment, if applicable, at each of the indicated visits.
^{m}CMP (comprehensive metabolic panel) includes BUN, CrCl, eGFR, sodium, calcium (corrected for serum albumin), potassium, chloride, phosphate, and magnesium, CO₂, fasting glucose, fasting amylase, fasting lipase, albumin, total protein, total and direct bilirubin, ALP, AST, ALT, GGT, LDH, and thyroid function tests (TSH and free T4). FSH and estradiol will be measured also at Screening for women under 60 years old (unless a documentation of prior bilateral oophorectomy is available).
ⁿLipid panel consists of total cholesterol, low-density lipoprotein (LDL), high-density lipoprotein (HDL), and triglycerides.
^{o}International normalized ratio (INR), prothrombin time (PT) and activated partial thromboplastin time (aPTT)
^{p}If abnormalities are present, microscopic testing should be done.
^{q}ADA samples must be collected prior to infusion.
^{r}If Hy's Law Criteria are met for a subject, a repeat blood draw will be taken approximately 72 hours later (between 48-72 hours) to confirm the LFT abnormalities and to determine if they are increasing or decreasing.
^{s}24-hour in-subject monitoring is required following the priming dose and the first full dose of cycle 1 in Phase 1a only.

Assessment of Safety: Safety assessments will consist of monitoring and recording all AEs, including all CTCAE v5.0 grades (for both increasing and decreasing severity); monitoring of hematology, complete metabolic panel, lipid panel, coagulation panel and urine assessments; periodic measurement of vital signs; ECOG performance status; ECGs; and performance of physical examinations as detailed in FIG. 6. All safety analyses will be performed using SAS version 9.4 or a later version.

Demographic/Medical History: Demographic information such as sex, age, race, ethnicity, as well as weight, height, and cancer-specific history and prior therapy status and medical and surgical history and current medical conditions will be recorded at the Screening Visit. All other clinically significant medical and surgical history within 5 years must be noted in the Medical History, Previous and Concomitant Medication and Procedures eCRF.

ECOG Performance Status: The Eastern Cooperative Oncology Group (ECOG) Performance Status Scale is presented in Table 15 below. ECOG performance status is to be recorded during screening, on Day 1 of each treatment cycle and on Day 28 safety follow up visit.

TABLE 16
ECOG Performance Status Criteria

| Grade | Description |
|---|---|
| 0 | Normal activity. Fully active, able to carry on all pre-disease |
| | performance without restriction. |
| 1 | Symptoms, but ambulatory. Restricted in physically strenuous |
| | activity, but ambulatory and able to carry out work of a light |
| | or sedentary nature (e.g. light housework, office work). |
| 2 | In bed <50% of the time. Ambulatory and capable of all self- |
| | care, but unable to carry out any work activities. Up and about |
| | more than 50% of waking hours. |
| 3 | In bed >50% of the time. Capable of only limited self-care, |
| | confined to bed or chair more than 50% of waking hours. |
| 4 | 100% bedridden. Completely disabled. Cannot carry on any |
| | self-care. Totally confined to bed or chair. |
| 5 | Dead |

Physical Examination and Vital Signs: A complete physical examination (including also neurological examination) will be performed at Screening, and throughout the study at each study visit. It will include a general appearance observation and a complete examination of the following body systems/areas: Head, Eyes, Ears, Nose and Throat (HEENT)/Neck, Lymph Nodes, Thyroid, Abdomen, Skin, Cardiovascular, Respiratory, Gastrointestinal, Neurological and Musculoskeletal/Extremities.

Vital signs will be recorded throughout the study at each study visit from Screening to the Safety Follow-up Visit. The following parameters will be measured:
Heart rate (beats/minute).
BP (systolic and diastolic, mmHg).
Respiratory rate (breaths/minute).
body temperature (° C.).

Body weight (to the nearest 0.1 kilogram in indoor clothing, but without shoes) will be measured during the Screening Period and at Day 1 of subsequent cycles. Height will be recorded at the baseline visit only.

No need to repeat physical examinations on C1/D1 if the baseline physical examination was performed within ≤72 hours.

Electrocardiogram (ECG): A standard 12-lead ECG will be used in this study. Each ECG, both at Screening and during the study, is to be performed in triplicate approximately 2 minutes apart after the subject has been semi-recumbent for at least 5 minutes. Each of the ECG parameters (heart rate, QTcF interval QTcB interval, PR interval, and QRS duration), values from each of the triplicate measurements will be entered in the eCRF. See more information in the schedule of assessment (TABLE 15).

Laboratory Assessments: All laboratory assessments will be performed as shown in the schedule of assessment (TABLE 15).

Hematology: Hematological parameters include the following laboratory tests: complete blood count (CBC) consisting of red blood cell count (RBC), hematocrit, hemoglobin, mean corpuscular volume (MCV), mean corpuscular hemoglobin, total white blood count (WBC) with 5-part differential, and platelet count plus reticulocyte count. Hematology assessment does not need to be repeated on C1/D1 if it was performed within 72 hours of baseline. CBCs should be collected and results assessed at screening and D1 of each cycle before treatment.

Comprehensive Metabolic Panel: The comprehensive metabolic panel (CMP) includes the following: blood urea nitrogen (BUN), creatinine, glomerular filtration (creatinine clearance), sodium, calcium (corrected for serum albumin), potassium, chloride, phosphate, and magnesium, carbon dioxide, fasting glucose, fasting amylase, fasting lipase, albumin, total protein, total and direct bilirubin, alkaline phosphatase (ALP), aspartate aminotransferase (AST), alanine aminotransferase (ALT), gamma-glutamyl transferase (GGT), lactate dehydrogenase, and thyroid function tests (TSH and Free T4). FSH and estradiol will be measured also at screening for women under 60 years old (unless a documentation of prior bilateral oophorectomy is available).

Lipid Panel: Lipid panel consists of total cholesterol, low-density lipoprotein (LDL), high-density lipoprotein (HDL), and triglycerides.

Coagulation: Prothrombin time (PT), partial thromboplastin time (PTT or activated PTT) and international normalized ratio (INR) will be assessed at each visit as per the table of assessments (TABLE 15).

Urinalysis: Urine analysis will be performed as per standard practice at the site. If abnormalities are present, microscopic testing should be done. An overall clinical judgment will be recorded in eCRF.

Pregnancy Screen: A serum pregnancy test must be performed during Screening (within 1 week of first dose), for all women of childbearing potential (i.e., pre- and perimenopausal subjects). Thereafter, during the study, urine testing should be performed on Day 1 of each cycle. Serum testing should be performed to confirm a positive urine test.

Pharmacokinetics and Anti-drug Antibodies Assessments: Pharmacokinetics sampling schedule is presented in TABLE 17 and TABLE 18, below, for the escalation and expansion phases, respectively. Anti-drug antibody (ADA) sampling is to be collected at the timepoints indicated in the schedule of assessment (TABLE 15), blood samples for ADA must be collected prior to infusion. Refer to the Laboratory Manual for details regarding PK and ADA sample collection, processing, storage, and shipment.

TABLE 17
Pharmacokinetic Sampling Schedule (Phase 1a)

| Visit | Day | Time (hour) | Window |
|---|---|---|---|
| | 1 (priming dose) | End of Infusion (EOI) | Up to 5 minutes post-infusion |
| | 2 | 24 | ±1 hours |
| | 8 (full cohort dose) | 0 (predose); serves as | Within 1 hour prior to dosing |
| | | 168 hours post Day 1 dose | |
| | | End of Infusion (EOI) | Up to 5 minutes post-infusion |
| | | 0.25 post-EOI | ±5 minutes |
| | | 1 post-EOI | ±10 minutes |
| | | 4 post-EOI | ±20 minutes |
| | 9 | 24 | ±1 hours |
| | 10 | 48 | ±2 hours |
| | 11 | 72 | ±2 hours |
| | 15 | 0 (predose); serves as | Within 1 hour prior to dosing |
| | | 168 hours post Day 8 | |
| | | End of Infusion (EOI) | Up to 5 minutes post-infusion |
| | 22 | 0 (predose) | Within 1 hour prior to dosing |
| | | End of Infusion (EOI) | Up to 5 minutes post-infusion |
| Cycle 2 | 1 | 0 (predose) | Within 1 hour prior to dosing |
| | | End of Infusion (EOI) | Up to 5 minutes post-infusion |
| | | 0.25 post-EOI | ±5 minutes |
| | | 1 post-EOI | ±10 minutes |
| | | 4 post-EOI | ±20 minutes |
| | 2 | 24 | ±1 hours |
| | 3 | 48 | ±2 hours |
| | 4 | 72 | ±2 hours |
| | 8 | 0 (predose); serves as | Within 1 hour prior to dosing |
| | | 168 hours post Day 1 dose | |
| Cycle 3+ | 1 | 0 (predose) | Within 1 hour prior to dosing |
| | | End of Infusion (EOI) | Up to 5 minutes post-infusion |

TABLE 18
Pharmacokinetic Sampling Schedule (Phase 1b)

| Visit | Day | Time (hour) | Window |
|---|---|---|---|
| Cycle 1 | 1 (priming) | 0 (predose) | Within 1 hour prior to dosing |
| | | End of Infusion (EOI) | Up to 5 minutes post-infusion |
| | 8 (full dose) | 0 (predose); serves as | Within 1 hour prior to dosing |
| | | 168 hours post-Day 1 dose | |
| | | End of Infusion (EOI) | Up to 5 minutes post-infusion |
| | | 4 post-EOI | ±20 minutes |
| | 15 | 0 (predose); serves as | Within 1 hour prior to dosing |
| | | 168 hours post-Day 8 dose | |
| | | End of Infusion (EOI) | Up to 5 minutes post-infusion |
| Cycle 2 | 1 | 0 (predose) | Within 1 hour prior to dosing |
| | | End of Infusion (EOI) | Up to 5 minutes post-infusion |
| | | 4 post-EOI | ±20 minutes |
| | 8 | 0 (predose); serves as | Within 1 hour prior to dosing |
| | | 168 hours post-Day 1 dose | |
| Cycle 3+ | 1 | 0 (predose) | Within 1 hour prior to dosing |
| | | End of Infusion (EOI) | Up to 5 minutes post-infusion |

### Pharmacodynamics Assessments

Serum Cytokines: To monitor immune activation over time the concentrations of pro-inflammatory cytokines including, but not limited to, IL-2, IL-6, IL-8, IL-10, MCP1, IFNγ, TNFα, GM-CSF and CRP will be examined according to the schedule presented in TABLE 19 below.

TABLE 19
Serum Cytokine Samples Collection Plan

| Day | Time point |
|---|---|
| Day 1 | Pre-infusion (all cycles) |
| Day 2 | 24 hours post infusion (cycle 1 only) |
| Day 8 | Pre-infusion (all cycles) |
| Day 15 (±1 day) | Pre-infusion (all cycles) |
| EOT | Day 28 Safety follow-up visit |

Modulation of The Immune System in Blood: Basic lymphocyte composition (T-, B- and NK-cells), T cell subsets and markers of activation and proliferation will also be monitored. The T phenotype will be determined using standard subset and activation markers (e.g., CD3, CD4, CD8, CD16, CD56, CD45RA, CCR7, CD27, CD25, CD69, TILA-DR, CD107A, and Ki67). In addition, T cell exhaustion will be followed using markers such as PD1, TIM3, and LAG3. In addition, the number and tumor spatial distribution of the immune cell subsets will be investigated (T, B, and NIK levels, and cell counts). Collections will be done according to the schedule presented in Table 20 below.

TABLE 20
Modulation of Immune System Sample Collection Plan

| Day | Time point |
|---|---|
| Day 1 | Pre-infusion (all cycles) |
| Day 2 | 24 hours post infusion (cycle 1 only) |
| Day 8 | Pre-infusion (all cycles) |
| Day 15 | Pre-infusion (all cycles) |
| End of Treatment | Day 28 Safety Follow-up Visit |

Changes Within the Tumor and Microenvironment: An optional FFPE will be requested at Week 8 (±1 week) after the priming dose of the bispecific antibody to evaluate changes in CLDN6 expression and tumor infiltrating lymphocytes (TILs).

### Example 2: Phase 1b Dose Expansion of CLDN6/CD3 Bispecific Antibody

The CLDN6/CD3 bispecific will be evaluated in one indication at up to 2 doses (n=15 response evaluable subjects at each dose level) in the dose expansion phase of the trial (Phase 1b) studying one of the following advanced solid tumor types: PRROC, Testicular, or Endometrial. This to enable dose- and exposure-response analyses. Dosing will follow the same schedule as Phase 1a (see Example 1 above). A priming dose will be administered on Day 1, and the full cohort dose will be administered weekly thereafter. Expansion doses for Phase 1b will be determined by Sponsor in conjunction with SRC based upon all available safety, PK, PD, biomarker and preliminary efficacy data from Phase 1a.

The selection of the recommended Phase 2 dose (RP2D) will be based on the totality of data from Phase 1b.

### Trial Objectives and Endpoints

The objectives and corresponding endpoints for Phase 1b are provided in Table 21 below:

TABLE 21
Phase 1b Dose Escalation Objectives and Endpoints

| | |
|---|---|
| Objective | Endpoint |

Phase 1b, Dose Expansion

| | |
|---|---|
| Primary Objectives | Endpoints |
| To evaluate preliminary efficacy of | ORR based on BOR assessed by Investigators |
| CLDN6/CD3 bispecific in subjects | according to RECIST v1.1. |
| with PRROC and other advanced | |
| CLDN6-positive solid tumors | |
| (testicular and endometrial). | |
| To further evaluate the safety of | Type, incidence, and severity of AEs, SAEs, AEs |
| CLDN6/CD3 bispecific. | of Special Interest, and laboratory abnormalities |
| | using the NCI CTCAE v.5.0. |
| | Frequency and duration of dose interruptions |
| | and reductions. |
| Secondary Objectives | Endpoints |
| To characterize the pharmacokinetic | PK parameters including, but not limited to |
| (PK) profile of CLDN6/CD3 | maximum concentration (Cₘₐₓ), time to Cₘₐₓ |
| bispecific. | (Tₘₐₓ), area under the curve (AUC), and terminal |
| | half-life (t_{1/2}). |
| To evaluate the | Assessment of anti-drug antibodies (ADA). |
| immunogenicity of | |
| CLDN6/CD3 bispecific. | |
| To evaluate efficacy of CLDN6/CD3 | Clinical Benefit Rate (CBR), DCR based on BOR, |
| bispecific. | DOR, Time to Progression (TTP) and Progression- |
| | free Survival (PFS) assessed by the Investigator |
| | according to RECIST v1.1. |
| | Overall Survival (OS). |
| To evaluate the efficacy of | iORR, iDCR, iCBR, iDOR, iTTP, iPFS |
| CLDN6/CD3 bispecific as per | assessed by Investigators according to |
| iRECIST. | iRECIST. |
| Exploratory Objective | Endpoints |
| To evaluate the relationship between | CLDN6 expression level at baseline and ORR, CBR, |
| CLDN6 expression levels and | PFS, iORR, iCBR and iPFS. |
| response to CLDN6/CD3 bispecific. | |
| Assess changes in pro- | Change over time of pro-inflammatory cytokines |
| inflammatory biomarkers. | (e.g., TNFα, IFNγ, GM-CSF, IL-1, IL-2, IL6, |
| | CRP, etc.). |
| Evaluate the PD effect of | Expression of markers of subset, activation, and |
| CLDN6/CD3 bispecific | proliferation by T cells (e.g. CD3, CD4, CD8, CD69, |
| monotherapy in modulating the | Ki67, etc.). |
| immune system in blood. | Number and kind of immune cell subsets such as T |
| | cells in circulation. |
| Assess changes within the tumor | Changes in CLDN6 expression. |
| and microenvironment. | Change in the number and kind of infiltrating |
| | lymphocytes (TILs). |

Based on the totality of data from Phase 1b, a recommended Phase 2 dose for future clinical studies will be determined.

### Example 3: Treatment of a Patient in Need Thereof with a Bispecific Antibody as Provided for Herein

A patient with a claudin 6 positive tumor is treated with a bispecific antibody comprising the polypeptides of SEQ ID NO: 18, SEQ ID NO: 17 and SEQ ID NO: 19 with an initial dose (priming dose) within the range (including the endpoints) of 140 μg to 1000 ug. One week later the patient is treated with a subsequent dose (full dose) within the range (including the endpoints) of 280 μg to 9000 ug. The patient is treated with a subsequent dose (full dose) weekly until the patient is administered a total of 6 doses including the initial dose. The patient is found to respond to treatment (priming dose followed by a higher dose) and the tumor regresses.

All references cited herein are incorporated by reference to the same extent as if each individual publication database entry (e.g. Genbank sequences of GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C. F. R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present embodiments are not to be limited in scope by the specific embodiments described herein. Indeed, various modifications in addition to those described herein will become apparent to those skilled in the art from the foregoing description and the accompanying figures. Such modifications are intended to fall within the scope of the embodiments and any appended claims.

The present specification is considered to be sufficient to enable one skilled in the art to practice the embodiments. Various modifications in addition to those shown and described herein will become apparent to those skilled in the art from the foregoing description and fall within the scope of the present disclosure and any appended claims.

1. A method of treating a Claudin 6 (CLDN6) expressing solid tumor cancer in a subject with pharmaceutical composition comprising a bispecific antibody that binds to CLDN6 and CD3, the method comprising:
administering a first dose of the pharmaceutical composition comprising the bispecific antibody; and
administering one or more subsequent doses of the pharmaceutical composition comprising the bispecific antibody,
wherein the first dose administered to the subject is less than the one or more subsequent doses administered to the subject.

2. The method of claim 1,
wherein the first dose of the bispecific antibody is selected from the group consisting of: about 10 μg to about 1500 μg, about 20 μg to about 1250 μg, about 22.5 μg to about 1000 μg, about 50 μg to about 800 μg, about 70 μg to about 750 μg, about 100 μg to about 500 μg, about 200 μg to about 400 μg, about 500 μg to about 1000 μg, about 600 μg to about 1000 μg, or about 800 μg to about 1000 μg, and
wherein the one or more subsequent dose of the bispecific antibody is selected from the group consisting of: about 10 μg to about 15000 μg, about 20 μg to about 13500 μg, about 22.5 μg to about 12800 μg, about 50 μg to about 10000 μg, about 70 μg to about 7500 μg, about 100 μg to about 6400 μg, about 200 μg to about 5000 μg, about 250 μg to about 4000 μg, about 300 μg to about 3200 μg, about 400 μg to about 2500 μg, about 500 μg to about 2000 μg, about 600 μg to about 1600 μg, about 800 μg to about 1000 μg, about 250 μg to about 10000 μg, about 550 μg to about 10000 μg, about 1100 μg to about 10000 μg, about 2000 μg to about 10000 μg, or about 4000 μg to about 10000 μg.

3. (canceled)

4. The method of claim 1, wherein the solid tumor cancer is ovarian cancer, testicular cancer, or endometrial cancer.

5.-8. (canceled)

9. The method of claim 1, wherein the bispecific antibody comprises a CLDN6 binding domain and a CD3 binding domain,
wherein the CLDN6 binding domain comprises a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and
wherein the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

10. The method of claim 9, wherein the CLDN6 binding domain comprises a first VH comprising an amino acid sequence having at least 90%, at least 95%, at least 98%, or 100% identity to the amino acid sequence of SEQ ID NO: 7, and comprises a first VL comprising an amino acid sequence having at least 90%, at least 95%, at least 98%, or 100% identity to the amino acid sequence of SEQ ID NO: 8.

11. The method of claim 9, wherein the CLDN6 binding domain comprises a heavy chain (HC) and a light chain (LC), wherein the HC comprises an amino acid sequence having at least 90%, at least 95%, at least 98%, or 100% identity to the amino acid sequence of SEQ ID NO: 17 and the LC comprises an amino acid sequence having at least 90%, at least 95%, at least 98%, or 100% identity to the amino acid sequence of SEQ ID NO: 18.

12. The method of claim 9, wherein the CD3 binding domain comprises a second VH comprising an amino acid sequence having at least 90%, at least 95%, at least 98%, or 100% identity to the amino acid sequence of SEQ ID NO: 15, and comprises a second VL comprising an amino acid sequence having at least 90%, at least 95%, at least 98%, or 100% identity to the amino acid sequence of SEQ ID NO: 16.

13. The method of claim 9, wherein the second VH and the second VL are linked by a polypeptide linker, optionally wherein the polypeptide linker comprises an amino acid sequence of SEQ ID NO: 20.

14. The method of claim 9, wherein the CD3 binding domain comprises an amino acid sequence having at least 90%, at least 85%, at least 98%, or 100% identity to the amino acid sequence of SEQ ID NO: 19.

15. The method of claim 9, wherein the bispecific antibody comprising a CLDN6 binding domain and a CD3 binding domain is a Fab-Fc-scFv, an IgG-scFv, or an IgG-(scFv)₂.

16. A method of treating a Claudin 6 (CLDN6) expressing solid tumor cancer in a subject in need thereof, comprising:
intravenously administering to the subject a first dose and one or more subsequent doses of a pharmaceutical composition comprising a bispecific antibody comprising a CLDN6 binding domain and a CD3 binding domain, wherein:
the first dose administered to the subject comprises about 70 μg to about 1000 μg of the bispecific antibody, and
the one or more subsequent doses administered to the subject comprises about 200 μg to about 12800 μg of the bispecific antibody,
wherein the CLDN6 binding domain comprises:
a first variable heavy chain region (VH) and a first variable light chain region (VL), wherein the first VH comprises a heavy chain complementarity domain region 1 (HCDR1) of SEQ ID NO: 1, a HCDR2 of SEQ ID NO: 2, and a HCDR3 of SEQ ID NO: 3, and the first VL comprises a light chain complementarity domain region (LCDR1) of SEQ ID NO: 4, a LCDR2 of SEQ ID NO: 5, and a LCDR3 of SEQ ID NO: 6; and
the CD3 binding domain comprises a second variable heavy chain region (VH) and a second variable light chain region (VL), wherein the second VH comprises a heavy chain complementarity domain region 1 (HCDR1) of SEQ ID NO: 9, a HCDR2 of SEQ ID NO: 10, and a HCDR3 of SEQ ID NO: 11, and the second VL comprises a light chain complementarity domain region (LCDR1) of SEQ ID NO: 12, a LCDR2 of SEQ ID NO: 13, and a LCDR3 of SEQ ID NO: 14.

17. (canceled)

18. The method of claim 16, wherein the solid tumor cancer is ovarian cancer, testicular cancer, endometrial cancer, or platinum refractory/resistant ovarian cancer PRROC.

19. (canceled)

20. The method of claim 16, wherein:
i) the first dose is about 70 μg and at least one or more of the one or more subsequent doses is about 140 μg;
ii) the first dose is about 70 μg and at least one or more of the one or more subsequent doses is about 200 μg;
iii) the first dose is about 140 μg and at least one or more of the one or more subsequent doses is about 280 μg;
iv) the first dose is about 200 μg and at least one or more of the one or more subsequent doses is about 400 μg;
v) the first dose is about 280 μg and at least one or more of the one or more subsequent doses is about 560 μg;
vi) the first dose is about 400 μg and at least one or more of the one or more subsequent doses is about 800 μg
vii) the first dose is about 560 μg and at least one or more of the one or more subsequent doses is about 1120 μg;
viii) the first dose is about 800 μg and at least one or more of the one or more subsequent doses is about 1600 μg;
ix) the first dose is about 1000 μg and at least one or more of the one or more subsequent doses is about 2240 μg;
x) the first dose is about 1000 μg and at least one or more of the one or more subsequent doses is about 3200 μg;
xi) the first dose is about 1000 μg and at least one or more of the one or more subsequent doses is about 4480 μg;
xii) the first dose is about 1000 μg and at least one or more of the one or more subsequent doses is about 6400 μg;
xiii) the first dose is about 1000 μg and at least one or more of the one or more subsequent doses is about 8960 μg; or
xiv) the first dose is about 1000 μg and at least one or more of the one or more subsequent doses is about 12800 μg.

21.-23. (canceled)

24. The method of claim 16, wherein the CLDN6 binding domain comprises a first VH comprising an amino acid sequence having at least 90%, at least 95%, at least 98%, or 100% identity to the amino acid sequence of SEQ ID NO: 7, and comprises a first VL comprising an amino acid sequence having at least 90%, at least 95%, at least 98%, or 100% identity to the amino acid sequence of SEQ ID NO: 8.

25. The method of claim 16, wherein the CLDN6 binding domain comprises a heavy chain (HC) and a light chain (LC), wherein the HC comprises an amino acid sequence having at least 90%, at least 95%, at least 98%, or 100% identity to the amino acid sequence of SEQ ID NO: 17 and the LC comprises an amino acid sequence having at least 90%, at least 95%, at least 98%, or 100% identity to the amino acid sequence of SEQ ID NO: 18.

26. The method of claim 16, wherein the CD3 binding domain comprises a second VH comprising an amino acid sequence having at least 90%, at least 95%, at least 98%, or 100% identity to the amino acid sequence of SEQ ID NO: 15, and comprises a second VL comprising an amino acid sequence having at least 90%, at least 95%, at least 98%, or 100% identity to the amino acid sequence of SEQ ID NO: 16.

27. (canceled)

28. The method of claim 16, wherein the CD3 binding domain comprises an amino acid sequence having at least 90%, at least 95%, at least 98%, or 100% identity to the amino acid sequence of SEQ ID NO: 19.

29. The method of claim 16, wherein the bispecific antibody comprising a CLDN6 binding domain and a CD3 binding domain is a Fab-Fc-scFv, an IgG-scFv, or an IgG-(scFv)₂.

30.-40. (canceled)

41. The method of claim 1, wherein:
i) administration of the first dose of the bispecific antibody and administration of the one or more subsequent doses of the bispecific antibody results in a Cₘₐₓ approximately equivalent to 0.04 nM;
ii) administration of the first dose of the bispecific antibody and administration of the one or more subsequent doses of the bispecific antibody results in a Cₘₐₓ of at least 0.04 nM;
iii) administration of the first dose of the bispecific antibody and administration of the one or more subsequent doses of the bispecific antibody results in an area under the curve (AUC) of approximately 175 hr*ng/mL;
iv) administration of the first dose of the bispecific antibody and administration of the one or more subsequent doses of the bispecific antibody results in an area under the curve (AUC) of at least 175 hr*ng/mL;
v) administration of the first dose of the bispecific antibody and administration of the one or more subsequent doses of the bispecific antibody results in minimal accumulation of anti-drug antibodies;
vi) administration of the first dose of the bispecific antibody and administration of the one or more subsequent doses of the bispecific antibody results in a complete response per RECIST Guidelines;
vii) administration of the first dose of the bispecific antibody and administration of the one or more subsequent doses of the bispecific antibody results in a partial response per RECIST Guidelines;
viii) administration of the first dose of the bispecific antibody and administration of the one or more subsequent doses of the bispecific antibody results in a stable disease response per RECIST Guidelines;
ix) administration of the first dose of the bispecific antibody and administration of the one or more subsequent doses of the bispecific antibody results in a complete response per Immune RECIST Guidelines;
x) administration of the first dose of the bispecific antibody and administration of the one or more subsequent doses of the bispecific antibody results in a partial response per Immune RECIST Guidelines;
xi) administration of the first dose of the bispecific antibody and administration of the one or more subsequent doses of the bispecific antibody results in a stable disease response per Immune RECIST Guidelines;
xii) administration of the first dose of the bispecific antibody and administration of the one or more subsequent doses of the bispecific antibody results in an increase in levels of one or more cytokines in the subject selected from IL-2, IL-6, IL-8, IL-10, MCP1, IFNγ, TNFα, GM-CSF and CRP;
xiii) administration of the first dose of the bispecific antibody and administration of the one or more subsequent doses of the bispecific antibody results in an increase in levels of one or more of CD3+ T cells, CD4+ T cells, CD*+ T cells, CD69+ T cells, or Ki67+ T cells in the subject;
xiv) administration of the first dose of the bispecific antibody and administration of the one or more subsequent doses of the bispecific antibody results in an increase in levels of one or more of T cells, B cells, natural killer (NK) cells, monocytes, macrophages, neutrophils, eosinophils, basophils, mast cells, or dendritic cells in the subject;
xv) administration of the first dose of the bispecific antibody and administration of the one or more subsequent doses of the bispecific antibody results in a decrease in CLDN6 expression in the tumor microenvironment;
xvi) administration of the first dose of the bispecific antibody and administration of the one or more subsequent doses of the bispecific antibody results in a change in the number and kind of infiltrating lymphocytes (TILs);
xvii) administration of the first dose of the bispecific antibody and administration of the one or more subsequent doses of the bispecific antibody results in a decrease in tumor size;
xviii) administration of the first dose of the bispecific antibody and administration of the one or more subsequent doses of the bispecific antibody results in depletion of tumor cells in the subject; and/or
xix) administration of the first dose of the bispecific antibody and administration of the one or more subsequent doses of the bispecific antibody results in an increased survival rate over a period of time as compared to subjects that do not receive the bispecific antibody.

42. The method of claim 16, wherein:
i) administration of the first dose of the bispecific antibody and administration of the one or more subsequent doses of the bispecific antibody results in a Cₘₐₓ approximately equivalent to 0.04 nM;
ii) administration of the first dose of the bispecific antibody and administration of the one or more subsequent doses of the bispecific antibody results in a Cₘₐₓ of at least 0.04 nM;
iii) administration of the first dose of the bispecific antibody and administration of the one or more subsequent doses of the bispecific antibody results in an area under the curve (AUC) of approximately 175 hr*ng/mL;
iv) administration of the first dose of the bispecific antibody and administration of the one or more subsequent doses of the bispecific antibody results in an area under the curve (AUC) of at least 175 hr*ng/mL;
v) administration of the first dose of the bispecific antibody and administration of the one or more subsequent doses of the bispecific antibody results in minimal accumulation of anti-drug antibodies;
vi) administration of the first dose of the bispecific antibody and administration of the one or more subsequent doses of the bispecific antibody results in a complete response per RECIST Guidelines;
vii) administration of the first dose of the bispecific antibody and administration of the one or more subsequent doses of the bispecific antibody results in a partial response per RECIST Guidelines;
viii) administration of the first dose of the bispecific antibody and administration of the one or more subsequent doses of the bispecific antibody results in a stable disease response per RECIST Guidelines;
ix) administration of the first dose of the bispecific antibody and administration of the one or more subsequent doses of the bispecific antibody results in a complete response per Immune RECIST Guidelines;
x) administration of the first dose of the bispecific antibody and administration of the one or more subsequent doses of the bispecific antibody results in a partial response per Immune RECIST Guidelines;
xi) administration of the first dose of the bispecific antibody and administration of the one or more subsequent doses of the bispecific antibody results in a stable disease response per Immune RECIST Guidelines;
xii) administration of the first dose of the bispecific antibody and administration of the one or more subsequent doses of the bispecific antibody results in an increase in levels of one or more cytokines in the subject selected from IL-2, IL-6, IL-8, IL-10, MCP1, IFNγ, TNFα, GM-CSF and CRP;
xiii) administration of the first dose of the bispecific antibody and administration of the one or more subsequent doses of the bispecific antibody results in an increase in levels of one or more of CD3+ T cells, CD4+ T cells, CD*+ T cells, CD69+ T cells, or Ki67+ T cells in the subject;
xiv) administration of the first dose of the bispecific antibody and administration of the one or more subsequent doses of the bispecific antibody results in an increase in levels of one or more of T cells, B cells, natural killer (NK) cells, monocytes, macrophages, neutrophils, eosinophils, basophils, mast cells, or dendritic cells in the subject;
xv) administration of the first dose of the bispecific antibody and administration of the one or more subsequent doses of the bispecific antibody results in a decrease in CLDN6 expression in the tumor microenvironment;
xvi) administration of the first dose of the bispecific antibody and administration of the one or more subsequent doses of the bispecific antibody results in a change in the number and kind of infiltrating lymphocytes (TILs);
xvii) administration of the first dose of the bispecific antibody and administration of the one or more subsequent doses of the bispecific antibody results in a decrease in tumor size;
xviii) administration of the first dose of the bispecific antibody and administration of the one or more subsequent doses of the bispecific antibody results in depletion of tumor cells in the subject; and/or
xix) administration of the first dose of the bispecific antibody and administration of the one or more subsequent doses of the bispecific antibody results in an increased survival rate over a period of time as compared to subjects that do not receive the bispecific antibody.

43. (canceled)
